SEQUENCE LISTING (47 sequences)

SEQ ID NO: 1 - protein, 4 aa, Artificial Sequence
  Synthetic
  misc_feature: see specification as filed for detailed description of substitutions and preferred embodiments
  misc_feature (1)..(1): X can be glycine (G), alanine (A), isoleucine (I), methionine (M), proline (P), tryptophan (W), tyrosine (Y)), serine (S), threonine (T), asparagine (N), glutamine (Q), histidine (H), lysine (K), arginine (R), aspartate (D), and glutamate (E)
XLQG

SEQ ID NO: 2 - protein, 4 aa, Artificial Sequence
  Synthetic
  misc_feature: see specification as filed for detailed description of substitutions and preferred embodiments
  misc_feature (2)..(2): X can be isoleucine (I), phenylalanine (F), threonine (T), glutamine (Q), histidine (H), arginine (R), and glutamate (E)
LXQG

SEQ ID NO: 3 - skipped

SEQ ID NO: 4 - protein, 9 aa, Artificial Sequence
  Synthetic
  misc_feature: see specification as filed for detailed description of substitutions and preferred embodiments
  misc_feature (4)..(4): Xaa can be any naturally occurring amino acid
GGSXLQGPP

SEQ ID NO: 5-6 - skipped (2 entries)

SEQ ID NO: 7 - protein, 9 aa, Artificial Sequence
  Synthetic
  misc_feature: see specification as filed for detailed description of substitutions and preferred embodiments
  misc_feature (5)..(5): Xaa can be any naturally occurring amino acid
GGSLXQGPP

SEQ ID NO: 8 - protein, 455 aa, Artificial Sequence
  Synthetic
MEVQLVESGGGLVQPGGSLRLSCAASGFNTTTKSIHWVRQAPGKGLEWVGEIYPRDGITDYADSVKGRFTISADTSKNTAYLQMNSLRAEDTAVYYCARGGWHWRSGYSYYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEAYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 215 aa, Artificial Sequence
  Synthetic
MDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 4 aa, Artificial Sequence
  Synthetic
GLQG

SEQ ID NO: 11 - protein, 4 aa, Artificial Sequence
  Synthetic
ALQG

SEQ ID NO: 12 - protein, 4 aa, Artificial Sequence
  Synthetic
VLQG

SEQ ID NO: 13 - protein, 4 aa, Artificial Sequence
  Synthetic
LLQG

SEQ ID NO: 14 - protein, 4 aa, Artificial Sequence
  Synthetic
ILQG

SEQ ID NO: 15 - protein, 4 aa, Artificial Sequence
  Synthetic
MLQG

SEQ ID NO: 16 - protein, 4 aa, Artificial Sequence
  Synthetic
PLQG

SEQ ID NO: 17 - protein, 4 aa, Artificial Sequence
  Synthetic
WLQG

SEQ ID NO: 18 - protein, 4 aa, Artificial Sequence
  Synthetic
FLQG

SEQ ID NO: 19 - protein, 4 aa, Artificial Sequence
  Synthetic
YLQG

SEQ ID NO: 20 - protein, 4 aa, Artificial Sequence
  Synthetic
SLQG

SEQ ID NO: 21 - protein, 4 aa, Artificial Sequence
  Synthetic
TLQG

SEQ ID NO: 22 - protein, 4 aa, Artificial Sequence
  Synthetic
NLQG

SEQ ID NO: 23 - protein, 4 aa, Artificial Sequence
  Synthetic
QLQG

SEQ ID NO: 24 - protein, 4 aa, Artificial Sequence
  Synthetic
HLQG

SEQ ID NO: 25 - protein, 4 aa, Artificial Sequence
  Synthetic
KLQG

SEQ ID NO: 26 - protein, 4 aa, Artificial Sequence
  Synthetic
RLQG

SEQ ID NO: 27 - protein, 4 aa, Artificial Sequence
  Synthetic
DLQG

SEQ ID NO: 28 - protein, 4 aa, Artificial Sequence
  Synthetic
ELQG

SEQ ID NO: 29 - protein, 4 aa, Artificial Sequence
  Synthetic
LGQG

SEQ ID NO: 30 - protein, 4 aa, Artificial Sequence
  Synthetic
LAQG

SEQ ID NO: 31 - protein, 4 aa, Artificial Sequence
  Synthetic
LVQG

SEQ ID NO: 32 - protein, 4 aa, Artificial Sequence
  Synthetic
LLQG

SEQ ID NO: 33 - protein, 4 aa, Artificial Sequence
  Synthetic
LIQG

SEQ ID NO: 34 - protein, 4 aa, Artificial Sequence
  Synthetic
LMQG

SEQ ID NO: 35 - protein, 4 aa, Artificial Sequence
  Synthetic
LPQG

SEQ ID NO: 36 - protein, 4 aa, Artificial Sequence
  Synthetic
LWQG

SEQ ID NO: 37 - protein, 4 aa, Artificial Sequence
  Synthetic
LFQG

SEQ ID NO: 38 - protein, 4 aa, Artificial Sequence
  Synthetic
LYQG

SEQ ID NO: 39 - protein, 4 aa, Artificial Sequence
  Synthetic
LSQG

SEQ ID NO: 40 - protein, 4 aa, Artificial Sequence
  Synthetic
LTQG

SEQ ID NO: 41 - protein, 4 aa, Artificial Sequence
  Synthetic
LNQG

SEQ ID NO: 42 - protein, 4 aa, Artificial Sequence
  Synthetic
LQQG

SEQ ID NO: 43 - protein, 4 aa, Artificial Sequence
  Synthetic
LHQG

SEQ ID NO: 44 - protein, 4 aa, Artificial Sequence
  Synthetic
LKQG

SEQ ID NO: 45 - protein, 4 aa, Artificial Sequence
  Synthetic
LRQG

SEQ ID NO: 46 - protein, 4 aa, Artificial Sequence
  Synthetic
LDQG

SEQ ID NO: 47 - protein, 4 aa, Artificial Sequence
  Synthetic
LEQG

(19) United States
(12) Patent Application Publication
(10) Pub. No.: US 2023/0095053 A1
(43) Pub. Date: Mar. 30, 2023

(54) ANTIBODIES COMPRISING SITE-SPECIFIC GLUTAMINE TAGS, METHODS OF THEIR PREPARATION AND METHODS OF THEIR USE

(71) Applicant: SUTRO BIOPHARMA, INC., South San Francisco, CA (US)

(72) Inventors: Junhao YANG, South San Francisco, CA (US); Jeffrey HANSON, South San Francisco, CA (US); Ryan STAFFORD, South San Francisco, CA (US); Gang YIN, South San Francisco, CA (US)

(21) Appl. No.: 17/908,882

(22) PCT Filed: Mar. 3, 2021
(86) PCT No.: PCT/US2021/020754

§ 371 Date: Sep. 1, 2022

Related U.S. Application Data
(60) Provisional application No. 62/984,698, filed on Mar. 3, 2020.

(51) Int. Cl. A61K 47/68; A61K 47/65; C07K 16/32

(52) U.S. Cl. CPC A61K 47/6803; A61K 47/65; A61K 47/6851; A61K 47/6889; C07K 16/32; C07K 2317/52; C07K 2317/526

(57) ABSTRACT

Provided herein are antibodies comprising acceptor glutamine sequences at site-specific positions, compositions comprising the antibodies, conjugates of the antibodies, methods of their production, and methods of their use. The antibodies are useful for methods of treatment and prevention, methods of detection and methods of diagnosis.

### FIELD

Provided herein are antibodies comprising glutamine mutations at site-specific positions, conjugates of the antibodies, compositions comprising the antibodies, methods of their production, and methods of their use. The antibodies and conjugates are useful, for example, for methods of treatment and methods of diagnosis.

### BACKGROUND

The efficacy of antibodies can be substantially enhanced by conjugation of payloads such as cytotoxic drugs, immunostimulatory compounds, and labelling groups. Conjugating to site-specific residues in antibodies has proved challenging. Conventional techniques include conjugation to random lysine or cysteine residues, with resulting heterogeneity and inefficiencies. Further techniques include antibodies engineered to include site-specific cysteine residues (Junutula et al., 2008, Nat. Biotechnol. 26:925-932) and site-specific incorporation of non-natural amino acids for conjugation (Kline et al., 2015, Pharm. Res. 32:3480-3493. In addition, bacterial transglutaminase has been used for site-specific drug conjugation at the heavy chain glutamine (Q295) residue of certain antibodies. Jeger et al., 2010, Angew. Chem. Int. Ed. 49:9995-9997. In later applications, certain peptide sequences, such as LLQG, have been engineered into antibodies at Q295 and other positions for drug conjugation. Strop et al., 2013, Chem. Biol. 20:161-167. While transglutaminase conjugation is promising, additional peptide sequences and new antibody sites for conjugation should provide useful opportunities for more efficient and stable conjugation. In 2008, several peptide sequences were identified as a potential substrate for Streptomyces mobaraensis transglutaminase in a phage-displayed random peptide library screen, including a 12 amino acid peptide M42 that was further characterized. Sugimura et al., 2008, Arch. Biochem. Biophys. 477:379-383. From the M42 peptide, at least five residues were identified as crucial for activity. See id.

To date, techniques for linking antibodies molecular entities such as molecular payloads, molecular shields, and labels have been limited by their heterogeneity in degree and location of linking to the antibodies, by their low yields and by losses in activity. In such techniques, some antibodies might be linked to the conjugate at one location while some antibodies are linked to the same conjugate at another location, and some antibodies might not be linked at all. There is a need for antibodies modified at site-specific positions optimized for uniformity, yield, and/or activity to further the promising use of antibodies in, for example, therapy and diagnostics.

### SUMMARY

Provided herein are antibodies comprising one or more non-naturally occurring sequences of amino acids that provide receptor glutamine residues for reaction with transglutaminase. In certain embodiments, these site-specific positions are optimal for substitution of natural amino acid residues with receptor glutamine sequences. In certain embodiments, substitution at these site-specific positions yields antibodies that are uniform in substitution, i.e. that are substantially modified in the selected position. In certain embodiments, an antibody substituted at these site-specific positions has advantageous production yield, advantageous solubility, advantageous binding, and/or advantageous activity. The properties of these antibodies are described in detail in the sections below.

In one aspect, provided herein are antibodies comprising one or more non-naturally occurring sequences of amino acids. The sequences comprise, consist essentially of, or consist of the sequence leucine (L)-glutamine (Q)-arginine (R) (i.e., LQR). In certain embodiments, one or more Q residues of the non-naturally occurring sequences is at a heavy chain site selected from 295, 296, 297, 120, 375, and 400. In certain embodiments, the antibody comprises the LQR sequence at one or more the following heavy chain sites, based upon EU or Kabat numbering: 294-295-296, 295-296-297, 296-297-298, 119-120-121, 374-375-376, and 399-400-401. In particular embodiments, the antibody comprises LQR at multiple sites. In some embodiments, one or more of the Q residues in the non-naturally occurring sequences is capable of accepting a primary amine in a transglutaminase reaction

In another aspect, the non-naturally occurring amino acids are at the C-terminus of an antibody heavy chain or light chain, and have the sequence X₁-leucine (L)-glutamine (Q)-glycine (G), wherein X₁ is an amino acid selected from the group consisting of glycine (G), alanine (A), isoleucine (I), methionine (M), proline (P), tryptophan (W), tyrosine (Y)), serine (S), threonine (T), asparagine (N), glutamine (Q), histidine (H), lysine (K), arginine (R), aspartate (D), and glutamate (E). Alternatively, the non-naturally occurring amino acids are at the C-terminus of an antibody heavy chain or light chain, and have the sequence leucine (L)-X₂-glutamine (Q)-glycine (G), wherein X₂ is an amino acid selected from the group consisting of isoleucine (I), phenylalanine (F), threonine (T), glutamine (Q), histidine (H), arginine (R), and glutamate (E).

In another aspect, provided herein are antibody conjugates comprising an antibody as described above linked to one or more therapeutic moieties, directly or via a linker bonded to one or more of the receptor glutamine residues. Some conjugates comprise an antibody linked to one or more drugs or polymers. Some conjugates comprise an antibody linked to one or more labeling moieties. Some conjugates comprise an antibody linked to one or more single chain binding domains (scFv). In some conjugates, at least one additional therapeutic or labeling moiety is also linked to said antibody via a site-specific non-natural amino acid residue. In some conjugates the antibody is linked to the additional moiety via a site specific para-azidophenylalanine or para-azidomethylphenylalanine residue.

In another aspect, provided herein is a composition comprising the antibody or antibody conjugate as described above having at least 95% by mass of the total antibody or antibody conjugate of said composition.

In another aspect, provided herein are methods of using the antibodies and antibody conjugates for therapy. These antibodies and antibody conjugates can be used for the treatment of cancer. Some antibodies and antibody conjugates may be used for the treatment of breast cancer. Some methods use a conjugate wherein at least one therapeutic moiety is linked via a glutamine residue in an LQR, X₁-L-Q-G, or L-X₂-Q-G sequence, and at least one additional therapeutic moiety is linked via a site-specific para-azidomethylphenylalanine or para-azidomethylphenylalanine residue. In some methods of treatment, the antibody conjugate comprises a drug. The drug may be useful for the treatment of cancer. In some embodiments, the drug is an auristatin, a maytansine, a hemiasterlin, or an immunostimulant. In some conjugates, the drug is a TLR agonist. Some conjugates used in methods of treatment as described herein comprise a labeling moiety. In some methods of treatment, a therapeutically effective amount of the conjugate is used.

In another aspect, provided herein is a C-terminus light chain library comprising one or more antibody light chains having the amino acid sequence -GGSX₁LQGPP or -GGSLX₂QGPP at the carboxy terminus of said light chains. In some embodiments, X₁ is a naturally or unnaturally occurring amino acid. In some embodiments X₁ is an amino acid selected from the group comprising G, A, I, M, P, W, Y, S, T, N, Q, H, K, R, D, and E. In some embodiments, X₂ is a naturally or unnaturally occurring amino acid. In some embodiment, X₂ is an amino acid selected from the group comprising I, F, T, Q, H, R and E.

### BRIEF DESCRIPTION OF THE FIGURES

FIG. 1 provides fluorescence intensity of a series of heavy chain LQR mutants conjugated with transglutaminase via glutamine residues to fluorescently labelled TAMRA-cadaverine, where residue number indicates the position of the leucine (L) residue of LQR.

FIG. 2 provides fluorescence intensity of a series of light chain LQR mutants conjugated with transglutaminase via glutamine residues to fluorescently labelled TAMRA-cadaverine, where residue number indicates the position of the leucine (L) residue of LQR.

FIG. 3 provides the normalized fluorescence intensity of C-term transglutaminase TAG library conjugated to TAMRA-cadavarine by transglutaminase from Streptomyces mobrensis (Zedria GmbH, ZsmTG) or Streptomyces hydroscopicus (shTG) at 10 and 1 U/mL. Fluorescence intensity is normalized to the LLQG transglutaminase consensus sequence. Data is ordered according to the mean relative fluorescence value across the four experiments.

FIG. 4 provides the mean fluorescence intensity of the transglutaminase tag library members sorted by residue type and position.

### DESCRIPTION OF EXEMPLARY EMBODIMENTS

Provided herein are antibodies having non-natural amino acids at one or more site-specific positions, compositions comprising the antibodies, methods of making the antibodies, and methods of their use.

### Definitions

When referring to the antibodies provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the singular forms “a,” “an,” and “the” include the plural reference unless the context clearly indicates otherwise. Thus, for example, reference to an “antibody” is a reference to one or more such antibodies, etc.

The term “substantially pure” with respect to a composition comprising an antibody refers to a composition that includes at least 80, 85, 90 or 95% by weight or, in certain embodiments, 95, 98, 99 or 100% by weight, e.g. dry weight, of the antibody relative to the remaining portion of the composition. The weight percentage can be relative to the total weight of protein in the composition or relative to the total weight of antibodies in the composition. Purity can be determined by techniques apparent to those of skill in the art, for instance SDS-PAGE.

The term “isolated” refers to an antibody that is substantially or essentially free of components that normally accompany or interact with the antibody as found in its naturally occurring environment or in its production environment, or both. Isolated antibody preparations have less than about 30%, less than about 25%, less than about 20%, less than about 15%, less than about 10%, less than about 5%, less than about 4%, less than about 3%, less than about 2% or less than about 1% of contaminating protein by weight, e.g. dry weight.

The term “antibody” refers to any macromolecule that would be recognized as an antibody by those of skill in the art. Antibodies share common properties including binding to an antigen and a structure comprising at least one polypeptide chain that is substantially identical to a polypeptide chain that can be encoded by any of the immunoglobulin genes recognized by those of skill in the art. The immunoglobulin genes include, but are not limited to, the κ, λ, α, γ (IgG1, IgG2, IgG3, and IgG4), δ, ε and μ constant region genes, as well as the immunoglobulin variable region genes (e.g., IGHV, IGHD, IGHJ, IGLV, IGKV, IGLJ, and IGKJ genes). The term includes full-length antibodies and antibody fragments recognized by those of skill in the art, and variants thereof.

The term “antibody fragment” refers to any form of an antibody other than the full-length form. Antibody fragments herein include antibodies that are smaller components that exist within full-length antibodies, and antibodies that have been engineered. Antibody fragments include but are not limited to Fv, Fc, Fab, and (Fab′)₂, single chain Fv (scFv), domain antibodies (dAbs), diabodies, triabodies, tetrabodies, bifunctional hybrid antibodies, CDR1, CDR2, CDR3, combinations of CDR's, variable regions, framework regions, constant regions, and the like (Maynard & Georgiou, 2000, Annu. Rev. Biomed. Eng. 2:339-76; Hudson, 1998, Curr. Opin. Biotechnol. 9:395-402).

The term “immunoglobulin (Ig)” refers to a protein consisting of one or more polypeptides substantially encoded by one or more of the immunoglobulin genes, or a protein substantially identical thereto in amino acid sequence. Immunoglobulins include but are not limited to antibodies. Immunoglobulins may have a number of structural forms, including but not limited to full-length antibodies, antibody fragments, and individual immunoglobulin domains including but not limited to V_{H}, D_{H}, J_{H}, C_{H} (e.g., Cγ1, Cγ2, Cγ3), V_{L}, J_{L}, and C_{L} (e.g., V_{κ} and V_{λ}).

The term “immunoglobulin (Ig) domain” refers to a protein domain consisting of a polypeptide substantially encoded by an immunoglobulin gene. Ig domains include but are not limited to V_{H}, D_{H}, J_{H}, C_{H} (e.g., Cγ1, Cγ2, Cγ3), V_{L}, J_{L}, and C_{L} (e.g., V_{κ} and V_{λ}).

The term “variable region” of an antibody refers to a polypeptide or polypeptides composed of the V_{H} immunoglobulin domain, the V_{L} immunoglobulin domains, or the V_{H} and V_{L} immunoglobulin domains. Variable region may refer to this or these polypeptides in isolation, or as a fragment (e.g., as an Fv fragment or as an scFv fragment), as this region in the context of a larger antibody fragment, or as this region in the context of a full-length antibody or an alternative, non-antibody scaffold molecule.

The term “variable” refers to the fact that certain portions of the variable domains differ extensively in sequence among antibodies and are responsible for the binding specificity of each particular antibody for its particular antigen. However, the variability is not evenly distributed through the variable domains of antibodies. It is concentrated in three segments called Complementarity Determining Regions (CDRs). Three of the CDRs are located in the light chain variable domain and three of the CDRs are located in the heavy chain variable domain. The more highly conserved portions of the variable domains are called the framework regions (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat et al., Sequences of Proteins of Immunological Interest, 5th Ed. Public Health Service, National Institutes of Health, Bethesda, Md. (1991)).

The constant domains are not typically involved directly in binding an antibody to an antigen, but exhibit various effector functions. Depending on the amino acid sequence of the constant region of their heavy chains, antibodies or immunoglobulins can be assigned to different classes. There are five major classes of immunoglobulins: IgA, IgD, IgE, IgG and IgM, and several of these may be further divided into subclasses (isotypes), e.g. IgG1, IgG2, IgG3, and IgG4; IgA1 and IgA2. The heavy chain constant regions that correspond to the different classes of immunoglobulins are called α, δ, ε, γ and μ, respectively. Of the various human immunoglobulin classes, only human IgG1, IgG2, IgG3 and IgM are known to activate complement.

The term “variant protein sequence” refers to a protein sequence that has one or more residues that differ in amino acid identity from another similar protein sequence. Said similar protein sequence may be the natural wild type protein sequence, or another variant of the wild type sequence. Variants include proteins that have one or more amino acid insertions, deletions, or substitutions. Variants also include proteins that have one or more post-translationally modified amino acids.

The term “parent antibody” refers to any antibody known to those of skill in the art that is modified according to the description provided herein. The modification can be physical, i.e., chemically or biochemically replacing or modifying one or more amino acids of the parent antibody to yield an antibody within the scope of the present description. The modification can also be conceptual, i.e., using the sequence of one or more polypeptide chains of the parent antibody to design an antibody comprising one or more site-specific non-natural amino acids according to the present description. Parent antibodies can be naturally occurring antibodies or antibodies designed or developed in a laboratory. Parent antibodies can also be artificial or engineered antibodies, e.g., chimeric or humanized antibodies.

The term “conservatively modified variant” refers to an antibody that differs from a related antibody by conservative substitutions in amino acid sequence. One of skill will recognize that individual substitutions, deletions or additions to a peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K), Histidine (H);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins: Structures and Molecular Properties (W H Freeman & Co.; 2nd edition (December 1993)

The terms “identical” or “identity,” in the context of two or more polypeptide sequences, refer to two or more sequences or subsequences that are the same. Sequences are “substantially identical” if they have a percentage of amino acid residues or nucleotides that are the same (i.e., about 60% identity, optionally about 65%, about 70%, about 75%, about 80%, about 85%, about 90%, about 95%, about 96%, about 97%, about 98%, about 99%, about 99.5%, or about 99.9% identity over a specified region), when compared and aligned for maximum correspondence over a comparison window, or designated region as measured using one of the following sequence comparison algorithms or by manual alignment and visual inspection. The identity can exist over a region that is at least about 50 amino acid residues or nucleotides in length, over a region that is about 10-17 residues in length (e.g., the approximate length of CDRL1), over a region that is about 7 residues in length (e.g., the approximate length of CDRL2), over a region that is about 7-11 residues in length (e.g., the approximate length of CDRL3), over a region that is about 10-12 residues in length (e.g., the approximate length of CDRH1), over a region that is about 16-19 residues in length (e.g., the approximate length of CDRH2), over a region that is about 3-35 residues in length (e.g., the approximate length of CDRH3), or over a region that is 75-100 amino acid residues or nucleotides in length, or, where not specified, across the entire sequence or a polypeptide. In the case of antibodies, identity can be measured outside the variable CDRs. Identity can also be measured within the entirety of the heavy or light chains, or within the variable regions of the heavy or light chains. Optimal alignment of sequences for comparison can be conducted, including but not limited to, by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection (see, e.g., Ausubel et al., Current Protocols in Molecular Biology (1995 supplement)).

Examples of algorithms that are suitable for determining percent sequence identity and sequence similarity include the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands. The BLAST algorithm is typically performed with the “low complexity” filter turned off.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “amino acid” refers to naturally occurring and non-naturally occurring amino acids, as well as imino acids such as proline, amino acid analogs, and amino acid mimetics that function in a manner similar to the naturally occurring amino acids.

Naturally encoded amino acids are the proteinogenic amino acids known to those of skill in the art. They include the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and the less common pyrrolysine and selenocysteine. Naturally encoded amino acids include post-translational variants of the 22 naturally occurring amino acids such as prenylated amino acids, isoprenylated amino acids, myrisoylated amino acids, palmitoylated amino acids, N-linked glycosylated amino acids, O-linked glycosylated amino acids, phosphorylated amino acids, and acylated amino acids.

The term “non-natural amino acid” refers to an amino acid that is not a proteinogenic amino acid, or a post-translationally modified variant thereof. In particular, the term refers to an amino acid that is not one of the 20 common amino acids or pyrrolysine or selenocysteine, or post-translationally modified variants thereof.

A “functional Releasing Factor 1 (RF1) protein” refers to RF1 that retains activity equal to or substantially similar to wild-type or unmodified RF1 protein. Functional RF1 activity can be tested, for example, by measuring the growth rate of bacteria expressing the modified RF1 protein, and comparing the growth rate to bacteria expressing wild-type or unmodified RF1. Functional RF1 activity can also be tested, for example, by the ability of the modified RF1 protein to reduce orthogonal tRNA incorporation of a nnAA at a specified position in an mRNA encoding a target protein, thereby increasing the amount of premature chain termination (i.e., increasing the amount of truncated protein).

An “attenuated Releasing Factor 1 (RF1) protein” refers to a modified RF1 that has reduced activity relative to wild-type or unmodified RF1 protein. RF1 activity can be tested, for example, by the ability of the modified RF 1 protein to reduce orthogonal tRNA incorporation of a nnAA at a specified position in an mRNA encoding a target protein, thereby increasing the amount of premature chain termination (i.e., increasing the amount of truncated protein). In some embodiments, the attenuated RF1 protein comprises transcriptional modifications; for example, the expression level of the RF1 protein (wild type or modified) can be reduced to achieve attenuation. The reduction can also achieved by using RNAi technologies. In some embodiments, the attenuated RF1 protein comprises translational modifications; for example, the amount of the synthesized RF1 protein (wild type or modified) can be reduced to achieve attenuation, e.g., by increasing the rate at which the protein is digested by protease via insertion of protease-specific sequence into the RF1 sequence.

The term “strained alkene” refers to a molecule comprising an alkene moiety that is capable of reacting with tetrazine in a tetrazine ligation. Exemplary tetrazine ligations are described in Blackman et al., 2008, J. Am. Chem. Soc. 130:13518-13519. Examples include trans-cyclooctenes and norbornenes. Useful compounds include, but are not limited to, trans-cyclooctene, (E)-cyclooct-4-enol, (E)-cyclooct-4-enyl 2,5-dioxo-1-pyrrolidinyl carbonate, 5-norbornene-2-acetic acid succinimidyl ester, and 5-norbornene-2-endo-acetic acid.

Antibodies

In one aspect, provided herein are antibodies comprising one or more glutamine residues in sequences at site-specific positions in one or more polypeptide chains. These site-specific positions are selected based on advantageous properties of the antibodies having glutamine residues at these positions. As described below, in particular embodiments, each such glutamine residue is within a three residue sequence of amino acids. The advantageous properties can relate to production yield, conjugation, solubility, binding, and/or activity. In certain embodiments, the three-residue sequence provides advantageous conjugation via a transglutaminase reaction. In certain embodiments, the three-residue sequence provides advantageous stability following conjugation. In certain embodiments, the site-specific positions described herein provide advantageous conjugation via a transglutaminase reaction. In certain embodiments, the site-specific positions provide advantageous stability to a conjugate following conjugation. In certain embodiments, the three residue sequence comprises, consists essentially of, or consists of the amino acids leucine (L), glutamine (Q), and arginine (R), in that order.

In certain embodiments, the antibody comprises one or more LQR sequences at site-specific positions. In certain embodiments, the antibody comprises two or more LQR sequences at site-specific positions. In certain embodiments, the antibody comprises three or more LQR sequences at site-specific positions. In certain embodiments, the antibody comprises four or more LQR sequences at site-specific positions. In certain embodiments, the antibody comprises five or more LQR sequences at site-specific positions. In certain embodiments, the antibody comprises six or more LQR sequences at site-specific positions.

In certain embodiments, each LQR sequence is independently at a specific site selected from the group consisting of optimally substitutable positions of any polypeptide chain of said antibody. These optimally substitutable positions are described in detail below. Exemplary optimally substitutable positions are also described below.

In certain embodiments, the antibody comprises one or more site-specific LQR sequences in a single heavy chain polypeptide. In certain embodiments, the antibody comprises two or more site-specific LQR sequences in a single heavy chain polypeptide. In certain embodiments, the antibody comprises three or more site-specific LQR sequences in a single heavy chain polypeptide. In certain embodiments, the antibody comprises four or more site-specific LQR sequences in a single heavy chain polypeptide. In certain embodiments, the antibody comprises five or more site-specific LQR sequences in a single heavy chain polypeptide. In certain embodiments, the antibody comprises six or more site-specific LQR sequences in a single heavy chain polypeptide.

In certain embodiments, the antibody comprises one to six site-specific LQR sequences in a single heavy chain polypeptide. In certain embodiments, the antibody comprises three to six site-specific LQR sequences in a single heavy chain polypeptide. In certain embodiments, the antibody comprises four to six site-specific LQR sequences in a single heavy chain polypeptide. In certain embodiments, the antibody comprises five to site-specific LQR sequences in a single heavy chain polypeptide. In certain embodiments, the antibody comprises one to four site-specific LQR sequences in a single heavy chain polypeptide. In certain embodiments, the antibody comprises one to three site-specific LQR sequences in a single heavy chain polypeptide. In certain embodiments, the antibody comprises one to two site-specific LQR sequences in a single heavy chain polypeptide. In certain embodiments, the antibody comprises three to six site-specific LQR sequences in a single heavy chain polypeptide. In certain embodiments, the antibody comprises three to five site-specific LQR sequences in a single heavy chain polypeptide. In certain embodiments, the antibody comprises three to four site-specific LQR sequences in a single heavy chain polypeptide.

In certain embodiments, the antibody comprises one or more site-specific LQR sequences in each of two heavy chain polypeptides. In certain embodiments, the antibody comprises two or more site-specific LQR sequences in each of two heavy chain polypeptides. In certain embodiments, the antibody comprises three or more site-specific LQR sequences in each of two heavy chain polypeptides. In certain embodiments, the antibody comprises four or more site-specific LQR sequences in each of two heavy chain polypeptides. In certain embodiments, the antibody comprises five or more site-specific LQR sequences in each of two heavy chain polypeptides. In certain embodiments, the antibody comprises six or more site-specific LQR sequences in each of two heavy chain polypeptides.

In certain embodiments, the antibody comprises one to six site-specific LQR sequences in each of two heavy chain polypeptides. In certain embodiments, the antibody comprises three to six site-specific LQR sequences in each of two heavy chain polypeptides. In certain embodiments, the antibody comprises four to six site-specific LQR sequences in each of two heavy chain polypeptides. In certain embodiments, the antibody comprises five to site-specific LQR sequences in each of two heavy chain polypeptides. In certain embodiments, the antibody comprises one to four site-specific LQR sequences in each of two heavy chain polypeptides. In certain embodiments, the antibody comprises one to three site-specific LQR sequences in each of two heavy chain polypeptides. In certain embodiments, the antibody comprises one to two site-specific LQR sequences in each of two heavy chain polypeptides. In certain embodiments, the antibody comprises three to six site-specific LQR sequences in each of two heavy chain polypeptides. In certain embodiments, the antibody comprises three to five site-specific LQR sequences in each of two heavy chain polypeptides. In certain embodiments, the antibody comprises three to four site-specific LQR sequences in each of two heavy chain polypeptides.

The LQR sequences are positioned at select locations in a polypeptide chain of the antibody. These locations were identified as providing optimum sites for substitution with the LQR sequences. Each site is capable of bearing an LQR sequence with optimum structure, function and/or methods for producing the antibody.

In another aspect, provided herein are antibodies comprising one or more glutamine residues in sequences at site-specific positions in one or more polypeptide chains. These site-specific positions are selected based on advantageous properties of the antibodies having glutamine residues at these positions. As described below, in particular embodiments, each such glutamine residue is within a four residue sequence of amino acids. The advantageous properties can relate to production yield, conjugation, solubility, binding, and/or activity. In certain embodiments, the four-residue sequence provides advantageous conjugation via a transglutaminase reaction. In certain embodiments, the four-residue sequence provides advantageous stability following conjugation. In certain embodiments, the site-specific positions described herein provide advantageous conjugation via a transglutaminase reaction. In certain embodiments, the site-specific positions provide advantageous stability to a conjugate following conjugation. In certain embodiments, the four residue sequence comprises, consists essentially of, or consists of the amino acids (X₁) (an amino acid acid), leucine (L), glutamine (Q), and glycine (G), in that order. In certain embodiments, for instance those consisting essentially of certain elements, the basic and novel features provided herein include some or all of: the X₁ sequences, their site-specific locations within an antibody, and transglutaminase conjugation. In other embodiments, provided herein are antibodies comprising X₁-L-Q-G at the C-terminus of one or more antibody light chains of the antibody, and transglutaminase conjugation. In other embodiments, provided herein are antibodies comprising X₁-L-Q-G at the C-terminus of one or more antibody heavy chains of the antibody, and transglutaminase conjugation. X₁ amino acids can be selected from the group consisting of G, A, I, M, P, W, Y, S, T, N, Q, H, K, R, D, and E.

In another aspect, provided herein are antibodies comprising one or more glutamine residues in sequences at site-specific positions in one or more polypeptide chains. These site-specific positions are selected based on advantageous properties of the antibodies having glutamine residues at these positions. As described below, in particular embodiments, each such glutamine residue is within a four residue sequence of amino acids. The advantageous properties can relate to production yield, conjugation, solubility, binding, and/or activity. In certain embodiments, the four-residue sequence provides advantageous conjugation via a transglutaminase reaction. In certain embodiments, the four-residue sequence provides advantageous stability following conjugation. In certain embodiments, the site-specific positions described herein provide advantageous conjugation via a transglutaminase reaction. In certain embodiments, the site-specific positions provide advantageous stability to a conjugate following conjugation. In certain embodiments, the four residue sequence comprises, consists essentially of, or consists of the amino acids leucine (L), (X₂) (an amino acid acid), glutamine (Q), and glycine (G), in that order. In certain embodiments, for instance those consisting essentially of certain elements, the basic and novel features provided herein include some or all of: the X₂ sequences, their site-specific locations within an antibody, and transglutaminase conjugation. In other embodiments, provided herein are antibodies comprising L-X₂-Q-G at the C-terminus of one or more light chains of the antibody, and transglutaminase conjugation. In other embodiments, provided herein are antibodies comprising L-X₂-Q-G at the C-terminus of one or more heavy chains of the antibody, and transglutaminase conjugation. X₂ amino acids can be selected from the group consisting of I, F, T, Q, H, R and E.

In certain embodiments, the antibody comprises the sequence X₁LQG in a single light chain polypeptide at the C-terminus. In certain embodiments, the antibody comprises X₁LQG sequences in two or more light chain polypeptides at their C-termini.

In certain embodiments, the antibody comprises the sequence X₁LQG in a single heavy chain polypeptide at the C-terminus. In certain embodiments, the antibody comprises X₁LQG sequences in two or more heavy chain polypeptides at their C-termini.

In certain embodiments, the antibody comprises the sequence LX₂QG in a single light chain polypeptide at the C-terminus. In certain embodiments, the antibody comprises LX₂QG sequences in two or more light chain polypeptides at their C-termini.

In certain embodiments, the antibody comprises the sequence LX₂QG in a single heavy chain polypeptide at the C-terminus. In certain embodiments, the antibody comprises LX₂QG sequences in two or more heavy chain polypeptides at their C-termini.

In certain embodiments, the antibody comprises the sequence GGSX₁LQGPP in a single light chain polypeptide at the C-terminus. In certain embodiments, the antibody comprises GGSX₁LQGPP sequences in two or more light chain polypeptides at their C-termini.

In certain embodiments, the antibody comprises the sequence GGSX₁LQGPP in a single heavy chain polypeptide at the C-terminus. In certain embodiments, the antibody comprises GGSX₁LQGPP sequences in two or more heavy chain polypeptides at their C-termini.

In certain embodiments, the antibody comprises the sequence GGSLX₂QGPP in a single light chain polypeptide at the C-terminus. In certain embodiments, the antibody comprises GGSLX₂QGPP sequences two or more light chain polypeptides at their C-termini.

In certain embodiments, the antibody comprises the sequence GGSLX₂QGPP in a single heavy chain polypeptide at the C-terminus. In certain embodiments, the antibody comprises GGSLX₂QGPP sequences two or more heavy chain polypeptides at their C-termini.

In certain embodiments, the antibody comprises one X₁LQG or LX₂QG sequence in a single light chain polypeptide.

In certain embodiments, the antibody comprises one X₁LQG or LX₂QG sequence in a single heavy chain polypeptide.

In certain embodiments, the antibody comprises X₁LQG or LX₂QG sequences at the C-termini in more than one light chain polypeptide of an antibody

In certain embodiments, the antibody comprises X₁LQG or LX₂QG sequences at the C-termini in more than one heavy chain polypeptide of an antibody.

In certain embodiments, the antibody comprises X₁LQG or LX₂QG sequences at the C-termini in each of two light chain polypeptides.

In certain embodiments, the antibody comprises X₁LQG or LX₂QG sequences at the C-termini in each of two heavy chain polypeptides.

In certain embodiments, the antibody comprises X₁LQG or LX₂QG sequences at the C-termini in at least one heavy chain polypeptide and at least one light chain polypeptide.

In certain embodiments, the antibody comprises X₁LQG or LX₂QG sequences at the C-termini in at least two heavy chain polypeptides and at least two light chain polypeptides.

In certain embodiments, the antibody comprises one GGSX₁LQGPP or GGSLX₂QGPP sequence in a single light chain polypeptide.

In certain embodiments, the antibody comprises one GGSX₁LQGPP or GGSLX₂QGPP sequence in a single heavy chain polypeptide.

In certain embodiments, the antibody comprises GGSX₁LQGPP or GGSLX₂QGPP sequences at the C-termini in more than one light chain polypeptide of an antibody.

In certain embodiments, the antibody comprises GGSX₁LQGPP or GGSLX₂QGPP sequences at the C-termini in more than one heavy chain polypeptide of an antibody.

In certain embodiments, the antibody comprises GGSX₁LQGPP or GGSLX₂QGPP sequences at the C-termini in each of two light chain polypeptides.

In certain embodiments, the antibody comprises GGSX₁LQGPP or GGSLX₂QGPP sequences at the C-termini in each of two heavy chain polypeptides.

In certain embodiments, the antibody comprises GGSX₁LQGPP or GGSLX₂QGPP sequences at the C-termini in each of one heavy chain polypeptide and one light chain polypeptide.

In certain embodiments, the antibody comprises GGSX₁LQGPP or GGSLX₂QGPP sequences at the C-termini in each of two heavy chain polypeptides and two light chain polypeptide.

In certain embodiments, for instance those consisting essentially of certain elements, the basic and novel features provided herein include some or all of: the LQR sequences, their site-specific locations within an antibody, and transglutaminase conjugation. In other embodiments, the basic and novel provided herein comprise some or all of X₁-L-Q-G or L-X₂-Q-G sequences at the C-terminus of the light antibody light chain of an antibody, and transglutaminase conjugation.

In certain embodiments, for instance those consisting essentially of certain elements, the basic and novel features provided herein include some or all of: the LQR sequences, their site-specific locations within an antibody, and transglutaminase conjugation. In other embodiments, the basic and novel provided herein comprise some or all of X₁-L-Q-G or L-X₂-Q-G sequences at the C-terminus of the heavy chain or light chain of an antibody, and transglutaminase conjugation

In certain embodiments, a site-specific position for substitution provides an antibody that is stable. Stability can be measured by any technique apparent to those of skill in the art. In certain embodiments, the substituted antibody or conjugate has a melting temperature that is within about 5° C. of the corresponding parent antibody, as described herein. In certain embodiments, the substituted antibody or conjugate has a melting temperature that is within about 4° C. of the corresponding parent antibody, as described herein. In certain embodiments, the substituted antibody or conjugate has a melting temperature that is within about 3° C. of the corresponding parent antibody, as described herein. In certain embodiments, the substituted antibody or conjugate has a melting temperature that is within about 2° C. of the corresponding parent antibody, as described herein. In certain embodiments, the substituted antibody or conjugate has a melting temperature that is within about 1° C. of the corresponding parent antibody, as described herein. In certain embodiments, the substituted antibody or conjugate has a melting temperature that is at least about 5° C. greater than the corresponding parent antibody, as described herein. In certain embodiments, the substituted antibody or conjugate has a melting temperature that is at least about 4° C. greater than the corresponding parent antibody, as described herein. In certain embodiments, the substituted antibody or conjugate has a melting temperature that is at least about 3° C. greater than the corresponding parent antibody, as described herein. In certain embodiments, the substituted antibody or conjugate has a melting temperature that is at least about 2° C. greater than the corresponding parent antibody, as described herein. In certain embodiments, the substituted antibody or conjugate has a melting temperature that is at least about 1° C. greater than the corresponding parent antibody, as described herein. The melting temperature can be Tm1, Tm2 or both Tm1 and Tm2 as will be recognized by those of skill in the art.

In certain embodiments, a site-specific position for substitution provides an antibody that is has optimal functional properties. For instance, the antibody can show little or no loss of binding affinity for its target antigen compared to an antibody without the site-specific non-natural amino acid. In certain embodiments, the antibody can show enhanced binding compared to an antibody without the site-specific non-natural amino acid.

In certain embodiments, a site-specific position for substitution provides an antibody that can be made advantageously. For instance, in certain embodiments, the antibody shows advantageous properties in its methods of synthesis, discussed below. In certain embodiments, the antibody can show little or no loss in yield in production compared to an antibody without the site-specific non-natural amino acid. In certain embodiments, the antibody can show enhanced yield in production compared to an antibody without the site-specific non-natural amino acid. In certain embodiments, the antibody can show little or no loss of tRNA suppression, described below, compared to an antibody without the site-specific non-natural amino acid. In certain embodiments, the antibody can show enhanced tRNA suppression, described below, in production compared to an antibody without the site-specific non-natural amino acid.

In certain embodiments, a site-specific position for substitution provides an antibody that has advantageous solubility. In certain embodiments, the antibody can show little or no loss in solubility compared to an antibody without the site-specific non-natural amino acid. In certain embodiments, the antibody can show enhanced solubility compared to an antibody without the site-specific non-natural amino acid.

In certain embodiments, a site-specific position for substitution provides an antibody that has advantageous expression. In certain embodiments, the antibody can show little or no loss in expression compared to an antibody without the site-specific non-natural amino acid. In certain embodiments, the antibody can show enhanced expression compared to an antibody without the site-specific non-natural amino acid.

In certain embodiments, a site-specific position for substitution provides an antibody that has advantageous folding. In certain embodiments, the antibody can show little or no loss in proper folding compared to an antibody without the site-specific non-natural amino acid. In certain embodiments, the antibody can show enhanced folding compared to an antibody without the site-specific non-natural amino acid.

In certain embodiments, a site-specific position for substitution provides an antibody that is capable of advantageous conjugation. As described below, several non-natural amino acids have side chains or functional groups that facilitate conjugation of the antibody to a second agent, either directly or via a linker. In certain embodiments, the antibody can show enhanced conjugation efficiency compared to an antibody without the same or other non-natural amino acids at other positions. In certain embodiments, the antibody can show enhanced conjugation yield compared to an antibody without the same or other non-natural amino acids at other positions. In certain embodiments, the antibody can show enhanced conjugation specificity compared to an antibody without the same or other non-natural amino acids at other positions.

The site-specific positions for substituting can be described with any antibody nomenclature system known to those of skill in the art. For convenience, the site can be identified by the position of the glutamine in the LQR sequence. In the Kabat numbering system, these positions are at EU heavy chain residues HC295, HC296, HC297, HC120, HC375, and HC400. In other words, provided herein are antibodies comprising LQR sequences positioning the Q of the LQR sequence at least one or more positions selected from EU heavy chain residues HC295, HC296, HC297, HC120, HC375, and HC400.

In certain embodiments, the antibodies comprise the mutations HC294L, HC295Q, and HC296R. In certain embodiments, the antibodies comprise the mutations HC295L, HC296Q, and HC297R. In certain embodiments, the antibodies comprise the mutations HC296L, HC297Q, and HC298R. In certain embodiments, the antibodies comprise the mutations HC119L, HC120Q, and HC121R. In certain embodiments, the antibodies comprise the mutations HC374L, HC375Q, and HC376R. In certain embodiments, the antibodies comprise the mutations HC399L, HC400Q, and HC401R. In each, numbering is according to Kabat or the EU numbering system of Kabat.

In certain embodiments, the antibodies comprise either the X₁-L-Q-G, or L-X₂-Q-G sequence at the C-terminus of the light chain(s). In some embodiments, X₁ is glycine (G), alanine (A), isoleucine (I), methionine (M), proline (P), tryptophan (W), tyrosine (Y)), serine (S), threonine (T), asparagine (N), glutamine (Q), histidine (H), lysine (K), arginine (R), aspartate (D), or glutamate (E). In some embodiments, X₂ is isoleucine (I), phenylalanine (F), threonine (T), glutamine (Q), histidine (H), arginine (R), and glutamate (E). In some embodiments X₁ or X₂ is not cysteine.

In certain embodiments, the antibodies comprise either the X₁-L-Q-G, or L-X₂-Q-G sequence at the C-terminus of the heavy chain(s). In some embodiments, X₁ is glycine (G), alanine (A), isoleucine (I), methionine (M), proline (P), tryptophan (W), tyrosine (Y)), serine (S), threonine (T), asparagine (N), glutamine (Q), histidine (H), lysine (K), arginine (R), aspartate (D), or glutamate (E). In some embodiments, X₂ is isoleucine (I), phenylalanine (F), threonine (T), glutamine (Q), histidine (H), arginine (R), and glutamate (E). In some embodiments X₁ or X₂ is not cysteine.

In some embodiments, the antibodies comprise more than one sequence motif selected from the group consisting of L-Q-R, X₁-L-Q-G, or L-X₂-Q-G. In some embodiments, the antibodies comprise one or more L-Q-R sequences in the heavy chain and X₁-L-Q-G at the C-terminus of at least one light chain. In some embodiments, the antibodies comprise one or more L-Q-R sequences in the heavy chain and L-X₂-Q-G at the C-terminus of at least one light chain. In some embodiments, the antibodies comprise one or more L-Q-R sequences in the heavy chain and GGSXLQGPP at the C-terminus of at least one light chain. In some embodiments, the antibodies comprise one or more L-Q-R sequences in the heavy chain and GGSLXQGPP at the C-terminus of at least one light chain.

In some embodiments, the antibodies comprise more than one sequence motif selected from the group consisting of L-Q-R, X₁-L-Q-G, or L-X₂-Q-G. In some embodiments, the antibodies comprise one or more L-Q-R sequences in the heavy chain and X₁-L-Q-G at the C-terminus of at least one heavy chain. In some embodiments, the antibodies comprise one or more L-Q-R sequences in the heavy chain and L-X₂-Q-G at the C-terminus of at least one light chain or one heavy chain. In some embodiments, the antibodies comprise one or more L-Q-R sequences in the heavy chain and GGSXLQGPP at the C-terminus of at least one light chain or one heavy chain. In some embodiments, the antibodies comprise one or more L-Q-R sequences in the heavy chain and GGSLXQGPP at the C-terminus of at least one light chain or one heavy chain.

In certain embodiments, a sequence is at a C-terminus when it is fused to the C-terminus of a polypeptide. In certain embodiments, a sequence is at a C-terminus when it is within 10, 9, 8, 7, 6, 5, 4, 3, 2, 1, or 0 residues of the C-terminus of a polypeptide. In certain embodiments, a sequence is at a C-terminus when it is within, 9, 8, 7, 6, 5, 4, 3, 2, 1, or 0 residues of the final numbered amino acid in an antibody polypeptide under an accepted numbering scheme, for instance Kabat or Chothia.

Surprisingly, at the −2 position, the amino acid residues G, A, I, M, P, W, Y, S, T, N, Q, H, K, R, D, and E showed similar or even greater activity compared to the consensus. Also surprisingly, at the −1 position, the amino acid residues I, F, T, Q, H, R, and E showed similar or even greater activity compared to the consensus.

In certain embodiments, the antibody comprises one or more further mutations. The mutations can, for instance, facilitate additional conjugation reactions. In certain embodiments, the antibodies comprise a Q295 mutation. In certain embodiments, the antibodies comprise a Q295A mutation. Such a mutation removes a native glutamine residue from the antibody sequence, thereby preventing a transglutaminase reaction at that site. In certain embodiments, the antibodies comprise a N297 mutation. Such a mutation removes a glycosylation site which can interfere with conjugation.

In certain embodiments, the antibody comprises one or more additional non-natural amino acids at other sites in the antibody. These additional non-natural amino acids can facilitate additional conjugation reactions, in addition to a transglutaminase conjugation at an LQR, X₁-L-Q-G, or L-X₂-Q-G sequence. In certain advantageous embodiments, these additional non-natural amino acids can facilitate additional conjugation reactions in the same reaction vessel with a transglutaminase reaction at an LQR, X₁-L-Q-G, or L-X₂-Q-G sequence in the same antibody. In certain embodiments, the additional site-specific non-natural amino acid residues are at sequence positions corresponding to residues selected from the group consisting of consisting of heavy chain or light chain residues HC404, HC121, HC180, LC22, LC7, LC42, LC152, HC136, HC25, HC40, HC119, HC190, HC222, HC19, HC52, HC70, HC110, and HC221. In certain embodiments, antibodies further comprise a non-natural amino acid residue at HC404. In certain embodiments, antibodies further comprise a non-natural amino acid residue at HC180. In certain embodiments, antibodies further comprise a non-natural amino acid residue at LC42.

The antibodies provided herein can be of any class or type known to those of skill in the art. In certain embodiments, the antibody can comprise a heavy chain of any type known to those of skill in the art. In certain embodiments, the antibody comprises a heavy chain of a type selected from the group consisting of α, δ, ε and μ. In certain embodiments, the antibody comprises an α heavy chain. In certain embodiments, the antibody comprises a δ heavy chain. In certain embodiments, the antibody comprises a ε heavy chain. In certain embodiments, the antibody comprises a μ heavy chain.

In certain embodiments, the antibody can comprise a light chain of any type known to those of skill in the art. In certain embodiments, the antibody comprises a light chain of a type selected from the group consisting of λ and κ. In certain embodiments, the antibody comprises a λ light chain. In certain embodiments, the antibody comprises a κ light chain.

Any of the above antibodies can be of any class known to those of skill in the art. In certain embodiments, the antibody is of a class or subclass selected from the group consisting of IgA, IgA1, IgA2, IgD, IgE, IgG, IgG1, IgG2, IgG3 and IgM. In certain embodiments, the antibody is an IgA antibody. In certain embodiments, the antibody is an IgA1 or an IgA2 antibody. In certain embodiments, the antibody is an IgD antibody. In certain embodiments, the antibody is an IgE antibody. In certain embodiments, the antibody is an IgG antibody. In certain embodiments, the antibody is an IgG1, IgG2, or IgG3 antibody. In certain embodiments, the antibody is an IgM antibody.

The antibody can be of any antibody form known to those of skill in the art. In certain embodiments, the antibody is an antibody fragment recognized by those of skill in the art. In certain embodiments, the antibody is an Fv, Fc, Fab or (Fab′)₂ antibody. In certain embodiments, the antibody is a single chain Fv antibody (scFv). In certain embodiments, the antibody is in the form of Fv, Fc, Fab, (Fab′)₂, single chain Fv (scFv) and/or scFv-Fc.

The antibody can share high sequence identity with any antibody recognized by those of skill in the art, i.e. a parent antibody. In certain embodiments, the amino acid sequence of the antibody is identical to the amino acid sequence of the parent antibody, other than the non-natural amino acids at site-specific position. In further embodiments, the antibody provided herein can have one or more insertions, deletions, or mutations relative to the parent antibody in addition to the one or more non-natural amino acids at the site-specific positions. In certain embodiments, the antibody provided herein can have a unique primary sequence, so long as it would be recognized as an antibody by those of skill in the art.

The antibody is typically a protein comprising multiple polypeptide chains. In certain embodiments, the antibody is a heterotetramer comprising two identical light (L) chains and two identical heavy (H) chains. Each light chain can be linked to a heavy chain by one covalent disulfide bond. Each heavy chain can be linked to the other heavy chain by one or more covalent disulfide bonds. Each heavy chain and each light chain can also have one or more intrachain disulfide bonds. As is known to those of skill in the art, each heavy chain typically comprises a variable domain (V_{H}) followed by a number of constant domains. Each light chain typically comprises a variable domain at one end (V_{L}) and a constant domain. As is known to those of skill in the art, antibodies typically have selective affinity for their target molecules, i.e. antigens.

In certain embodiments, further provided herein are conservatively modified variants of the above antibodies. Conservatively modified variants of an antibody include one or more insertions, deletions, or substitutions that do not disrupt the structure and/or function of the antibody when evaluated by one of skill in the art. In certain embodiments, conservatively modified variants include 20 or fewer amino acid insertions, deletions, or substitutions. In certain embodiments, conservatively modified variants include 15 or fewer amino acid insertions, deletions, or substitutions. In certain embodiments, conservatively modified variants include 10 or fewer amino acid insertions, deletions, or substitutions. In certain embodiments, conservatively modified variants include 9 or fewer amino acid insertions, deletions, or substitutions. In certain embodiments, conservatively modified variants include 8 or fewer amino acid insertions, deletions, or substitutions. In certain embodiments, conservatively modified variants include 7 or fewer amino acid insertions, deletions, or substitutions. In certain embodiments, conservatively modified variants include 6 or fewer amino acid insertions, deletions, or substitutions. In certain embodiments, conservatively modified variants include 5 or fewer amino acid insertions, deletions, or substitutions. In certain embodiments, conservatively modified variants include 4 or fewer amino acid insertions, deletions, or substitutions. In certain embodiments, conservatively modified variants include 3 or fewer amino acid insertions, deletions, or substitutions. In certain embodiments, conservatively modified variants include 2 or fewer amino acid insertions, deletions, or substitutions. In certain embodiments, conservatively modified variants include 1 amino acid insertion, deletion, or substitution. In particular embodiments the substitutions are conservative, substituting an amino acid within the same class, as described above.

In certain embodiments, the antibodies can be modified to modulate structure, stability, and/or activity. In such embodiments, the modifications can be conservative or other than conservative. The modifications need only be suitable to the practitioner carrying out the methods and using the compositions described herein. In certain embodiments, the modifications decrease but do not eliminate antigen binding affinity. In certain embodiments, the modifications increase antigen binding affinity. In certain embodiments, the modifications enhance structure or stability of the antibody. In certain embodiments, the modifications reduce but do not eliminate structure or stability of the antibody. In certain embodiments, modified variants include 20 or fewer amino acid insertions, deletions, or substitutions. In certain embodiments, modified variants include 15 or fewer amino acid insertions, deletions, or substitutions. In certain embodiments, modified variants include 10 or fewer amino acid insertions, deletions, or substitutions. In certain embodiments, modified variants include 9 or fewer amino acid insertions, deletions, or substitutions. In certain embodiments, modified variants include 8 or fewer amino acid insertions, deletions, or substitutions. In certain embodiments, modified variants include 7 or fewer amino acid insertions, deletions, or substitutions. In certain embodiments, modified variants include 6 or fewer amino acid insertions, deletions, or substitutions. In certain embodiments, modified variants include 5 or fewer amino acid insertions, deletions, or substitutions. In certain embodiments, modified variants include 4 or fewer amino acid insertions, deletions, or substitutions. In certain embodiments, modified variants include 3 or fewer amino acid insertions, deletions, or substitutions. In certain embodiments, modified variants include 2 or fewer amino acid insertions, deletions, or substitutions. In certain embodiments, modified variants include 1 amino acid insertion, deletion, or substitution.

Also within the scope are post-translationally modified variants. Any of the antibodies provided herein can be post-translationally modified in any manner recognized by those of skill in the art. Typical post-translational modifications for antibodies include interchain disulfide bonding, intrachain disulfide bonding, N-linked glycosylation, and proteolysis. Also provided herein are other post-translationally modified antibodies having modifications such as phosphorylation, O-linked glycosylation, methylation, acetylation, lipidation, GPI anchoring, myristoylation and prenylation. The post-translational modification can occur during production, in vivo, in vitro, or otherwise. In certain embodiments, the post-translational modification can be an intentional modification by a practitioner, for instance, using the methods provided herein.

Further included within the scope are antibodies fused to further peptides or polypeptides. Exemplary fusions include, but are not limited to, e.g., a methionyl antibody in which a methionine is linked to the N-terminus of the antibody resulting from the recombinant expression, fusions for the purpose of purification (including but not limited to, to poly-histidine or affinity epitopes), fusions for the purpose of linking to other biologically active molecules, fusions with serum albumin binding peptides, and fusions with serum proteins such as serum albumin. The antibodies may comprise protease cleavage sequences, reactive groups, antibody-binding domains (including but not limited to, FLAG or poly-His) or other affinity based sequences (including but not limited to, FLAG, poly-His, GST, etc.). The antibodies may also comprise linked molecules (including but not limited to, biotin) that improve detection (including but not limited to, GFP), purification or other features of the antibody. In certain embodiments, the antibodies comprise a C-terminal affinity sequence that facilitates purification of full length antibodies. In certain embodiments, such C-terminal affinity sequence is a poly-His sequence, e.g., a 6-His sequence.

The antibody can have any antibody form recognized by those of skill in the art. The antibody can comprise a single polypeptide chain—a single heavy chain or a single light chain. The antibody can also form multimers that will be recognized by those of skill in the art including homodimers, heterodimers, homomultimers, and heteromultimers. These multimers can be linked or unlinked. Useful linkages include interchain disulfide bonds typical for antibody molecules. The multimers can also be linked by other amino acids, including the non-natural amino acids introduced according to the present description. The antibody can be an immunoglobulin such as of any class or subclass including IgA, IgA1, IgA2, IgD, IgE, IgG, IgG1, IgG2, IgG3, IgG4, and IgM. The antibody can be of the form of any antibody fragment including Fv, Fc, Fab, and (Fab′)₂, and scFv.

Also provided herein are antibodies that are conjugated to one or more conjugation moieties. The conjugation moiety can be any conjugation moiety deemed useful to one of skill in the art. For instance, the conjugation moiety can be a polymer, such as polyethylene glycol, that can improve the stability of the antibody in vitro or in vivo. The conjugation moiety can have therapeutic activity, thereby yielding an antibody-drug conjugate. The conjugation moiety can be a molecular payload that is harmful to target cells. The conjugation moiety can be a label useful for detection or diagnosis. In certain embodiments, the conjugation moiety is linked to the antibody via a direct covalent bond. In certain embodiments, the conjugation moiety is linked to the antibody via a linker. In advantageous embodiments, the conjugation moiety or the linker is attached via one of the non-natural amino acids of the antibody. Exemplary conjugation moieties and linkers are discussed in the sections below.

Non-Natural Amino Acids

In certain embodiments, the antibodies further comprise one or more site-specific non-natural amino acids. These additional non-natural amino acids can facilitate conjugation to two or more different payloads, linkers, or linker-payloads. The non-natural amino acid can be any non-natural amino acid known to those of skill in the art. In some embodiments, the non-naturally encoded amino acid comprises a functional group. The functional group can be any functional group known to those of skill in the art. In certain embodiments the functional group is a label, a polar group, a non-polar group, or a reactive group.

Reactive groups are particularly advantageous for linking further functional groups to the antibody at the site-specific position of the antibody chain. In certain embodiments, the reactive group is selected from the group consisting of amino, carboxy, acetyl, hydrazino, hydrazido, semicarbazido, sulfanyl, azido, tetrazine, and alkynyl.

In certain embodiments, the amino acid residue is according to any of the following formulas:

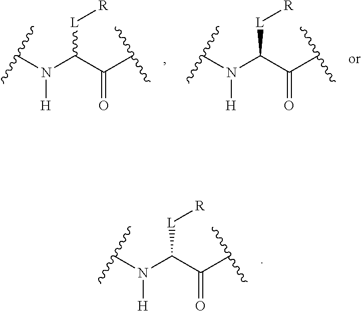

Those of skill in the art will recognize that antibodies are generally comprised of L-amino acids. However, with non-natural amino acids, the present methods and compositions provide the practitioner with the ability to use L-, D- or racemic non-natural amino acids at the site-specific positions. In certain embodiments, the non-natural amino acids described herein include D-versions of the natural amino acids and racemic versions of the natural amino acids.

In the above formulas, the wavy lines indicate bonds that connect to the remainder of the polypeptide chains of the antibodies. These non-natural amino acids can be incorporated into polypeptide chains just as natural amino acids are incorporated into the same polypeptide chains. In certain embodiments, the non-natural amino acids are incorporated into the polypeptide chain via amide bonds as indicated in the formulas.

In the above formulas R designates any functional group without limitation, so long as the amino acid residue is not identical to a natural amino acid residue. In certain embodiments, R can be a hydrophobic group, a hydrophilic group, a polar group, an acidic group, a basic group, a chelating group, a reactive group, a therapeutic moiety, or a labeling moiety. In certain embodiments, R is selected from the group consisting of R¹NR²R³, R¹C(═O)R², R¹C(═O)OR², R¹N3, R¹C(≡CH). In these embodiments, R¹ is selected from the group consisting of a bond, alkylene, heteroalkylene, arylene, heteroarylene. R² and R³ are each independently selected from the group consisting of hydrogen, alkyl, and heteroalkyl.

In some embodiments, the non-naturally encoded amino acids include side chain functional groups that react efficiently and selectively with functional groups not found in the 20 common amino acids (including but not limited to, azido, ketone, aldehyde and aminooxy groups) to form stable conjugates. For example, an antigen-binding polypeptide that includes a non-naturally encoded amino acid containing an azido functional group can be reacted with a polymer (including but not limited to, poly(ethylene glycol)) or, alternatively, a second polypeptide containing an alkyne moiety to form a stable conjugate resulting from the selective reaction of the azide and the alkyne functional groups to form a Huisgen [3+2]cycloaddition product. An antigen-binding polypeptide that includes a non-naturally encoded amino acid containing a tetrazine functional group can be reacted with a polymer (including but not limited to, poly(ethylene glycol)) containing a strained alkene moiety to form a stable conjugate resulting from the selective reaction of the tetrazine and strained alkene. Alternatively, a second polypeptide containing a strained alkene moiety may be reacted with the amino acid containing tetrazine functionality to form a stable conjugate resulting from the selective reaction of the tetrazine and strained alkene.

Exemplary non-naturally encoded amino acids that may be suitable for use in the present antibodies include, but are not limited to, those with carbonyl, aminooxy, hydrazine, hydrazide, semicarbazide, azide, and alkyne reactive groups. In some embodiments, non-naturally encoded amino acids comprise a saccharide moiety. Examples of such amino acids include N-acetyl-L-glucosaminyl-L-serine, N-acetyl-L-galactosaminyl-L-serine, N-acetyl-L-glucosaminyl-L-threonine, N-acetyl-L-glucosaminyl-L-asparagine, and O-mannosaminyl-L-serine. Examples of such amino acids also include examples where the naturally-occurring N- or O-linkage between the amino acid and the saccharide is replaced by a covalent linkage not commonly found in nature-including but not limited to, an alkene, an oxime, a thioether, an amide and the like. Examples of such amino acids also include saccharides that are not commonly found in naturally-occurring proteins such as 2-deoxy-glucose, 2-deoxygalactose, and the like.

Many unnatural amino acids are based on natural amino acids, such as tyrosine, glutamine, phenylalanine, and the like, and are suitable for use in the present invention. Tyrosine analogs include, but are not limited to, para-substituted tyrosines, ortho-substituted tyrosines, and meta substituted tyrosines, where the substituted tyrosine comprises, including but not limited to, a keto group (including but not limited to, an acetyl group), a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, a nitro group, an alkynyl group or the like. In addition, multiply substituted aryl rings are also contemplated. Glutamine analogs that may be suitable for use in the present invention include, but are not limited to, α-hydroxy derivatives, γ-substituted derivatives, cyclic derivatives, and amide substituted glutamine derivatives. Example phenylalanine analogs that may be suitable for use in the present invention include, but are not limited to, para-substituted phenylalanines, ortho-substituted phenylalanines, and meta-substituted phenylalanines, where the substituent comprises, including but not limited to, a hydroxy group, a methoxy group, a methyl group, an allyl group, an aldehyde, an azido, an iodo, a bromo, a keto group (including but not limited to, an acetyl group), a benzoyl, an alkynyl group, or the like. Specific examples of unnatural amino acids that may be suitable for use in the present invention include, but are not limited to, a p-acetyl-L-phenylalanine, an O-methyl-L-tyrosine, an L-3-(2-naphthyl)alanine, a 3-methyl-phenylalanine, an O-4-allyl-L-tyrosine, a 4-propyl-L-tyrosine, a tri-O-acetyl-GlcNAcβ-serine, an L-Dopa, a fluorinated phenylalanine, an isopropyl-L-phenylalanine, a p-azido-L-phenylalanine, p-azido-methyl-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, an isopropyl-L-phenylalanine, and a p-propargyloxy-phenylalanine, and the like. Examples of structures of a variety of unnatural amino acids that may be suitable for use in the present invention are provided in, for example, WO 2002/085923 entitled “In vivo incorporation of unnatural amino acids.” See also Kiick et al., (2002) Incorporation of azides into recombinant proteins for chemoselective modification by the Staudinger ligation, PNAS 99:19-24, for additional methionine analogs. In particular embodiments, the non-natural amino acid is p-azido-L-phenylalanine. In particular embodiments, the non-natural amino acid is p-azido-methyl-L-phenylalanine.

Many of the unnatural amino acids suitable for use in the present antibodies are commercially available, e.g., from Sigma (USA) or Aldrich (Milwaukee, Wis., USA). Those that are not commercially available are optionally synthesized as provided herein or as provided in various publications or using standard methods known to those of skill in the art. For organic synthesis techniques, see, e.g., Organic Chemistry by Fessendon and Fessendon, (1982, Second Edition, Willard Grant Press, Boston Mass.); Advanced Organic Chemistry by March (Third Edition, 1985, Wiley and Sons, New York); and Advanced Organic Chemistry by Carey and Sundberg (Third Edition, Parts A and B, 1990, Plenum Press, New York). Additional publications describing the synthesis of unnatural amino acids include, e.g., WO 2002/085923 entitled “In vivo incorporation of Unnatural Amino Acids;” Matsoukas et al., (1995) J. Med. Chem., 38, 4660-4669; King, F. E. & Kidd, D. A. A. (1949) A New Synthesis of Glutamine and of y-Dipeptides of Glutamic Acid from Phthylated Intermediates. J. Chem. Soc., 3315-3319; Friedman, O. M. & Chatterrji, R. (1959) Synthesis of Derivatives of Glutamine as Model Substrates for Anti-Tumor Agents. J. Am. Chem. Soc. 81, 3750-3752; Craig, J. C. et al. (1988) Absolute Configuration of the Enantiomers of 7-Chloro-4 [[4-(diethylamino)-1-methylbutyl]amino]quinoline (Chloroquine). J. Org. Chem. 53, 1167-1170; Azoulay, M., Vilmont, M. & Frappier, F. (1991) Glutamine analogues as Potential Antimalarials, Eur. J. Med. Chem. 26, 201-5; Koskinen, A. M. P. & Rapoport, H. (1989) Synthesis of 4-Substituted Prolines as Conformationally Constrained Amino Acid Analogues. J. Org. Chem. 54, 1859-1866; Christie, B. D. & Rapoport, H. (1985) Synthesis of Optically Pure Pipecolates from L-Asparagine. Application to the Total Synthesis of (+)-Apovincamine through Amino Acid Decarbonylation and Iminium Ion Cyclization. J. Org. Chem. 1989:1859-1866; Barton et al., (1987) Synthesis of Novel a-Amino-Acids and Derivatives Using Radical Chemistry: Synthesis of L- and D-a-Amino-Adipic Acids, L-a-aminopimelic Acid and Appropriate Unsaturated Derivatives. Tetrahedron Lett. 43:4297-4308; and, Subasinghe et al., (1992) Quisqualic acid analogues: synthesis of beta-heterocyclic 2-aminopropanoic acid derivatives and their activity at a novel quisqualate-sensitized site. J. Med. Chem. 35:4602-7. See also, patent applications entitled “Protein Arrays,” filed Dec. 22, 2003, Ser. No. 10/744,899 and Ser. No. 60/435,821 filed on Dec. 22, 2002.

In particular embodiments, the non-natural amino acids are selected from p-acetyl-phenylalanine, p-ethynyl-phenylalanine, p-propargyloxyphenylalanine, p-azido-phenylalanine, and p-azido-methyl-phenylalanine. One particularly useful non-natural amino acid is p-azido-phenylalanine. Another particularly useful non-natural amino acid is p-azido-methyl-phenylalanine (U.S. Pat. No. 9,682,934). These amino acid residues are known to those of skill in the art to facilitate Huisgen [3+2] cycloaddition reactions (so-called “click” chemistry reactions) with, for example, compounds bearing alkynyl groups. This reaction enables one of skill in the art to readily and rapidly conjugate to the antibody at the site-specific location of the non-natural amino acid.

In certain embodiments, the first reactive group is an alkynyl moiety (including but not limited to, in the unnatural amino acid p-propargyloxyphenylalanine, where the propargyl group is also sometimes referred to as an acetylene moiety) and the second reactive group is an azido moiety, and [3+2] cycloaddition chemistry can be used. In certain embodiments, the first reactive group is the azido moiety (including but not limited to, in the unnatural amino acid p-azido-L-phenylalanine) and the second reactive group is the alkynyl moiety.

In certain embodiments, the non-natural amino acids comprise tetrazine functional groups. Incorporation of tetrazine functional groups in non-natural amino acids enables selective and efficient reaction of the non-natural amino acids with compounds comprising strained alkenes. Useful strained alkenes include trans-cyclooctenes and norbornenes described herein. These reactions are selective in that the reactive groups—the tetrazines and the strained alkenes—are not reactive with the functional groups of the naturally occurring amino acids or with other well-known reactive groups. Further, the reactions can be carried out in complex environments such as cell extracts, in vitro protein synthesis reaction mixtures and the like.

The reaction between tetrazine and a strained alkene is known as the “tetrazine ligation.” It is believed that the tetrazine and strained alkene react in an inverse-demand Diels-Alder reaction followed by a retro-Diels-Alder reaction that links the tetrazine to the strained alkene. The reaction is specific, with little to no cross-reactivity with functional groups that occur on biomolecules. The reaction may be carried out under mild conditions, for example at room temperature and without a catalyst.

Conjugates, Linkers and Payloads

The antibodies provided herein are particularly useful for preparing antibody conjugates. In certain embodiments, provided are antibody conjugates comprising a payload linked to a glutamine residue of an LQR, X₁-L-Q-G, or L-X₂-Q-G sequence described herein. The linkage can be direct or by way of a linker. In certain embodiments, the antibody conjugates comprise more than one payloads or linker-payloads, or a combination thereof, linked to glutamine residues of LQR, X₁-L-Q-G, or L-X₂-Q-G sequences.

In certain embodiments, the antibody conjugates further comprise one or more additional payloads, or linker-payloads, linked to a site-specific non-natural amino acid as described herein. In certain embodiments, the site-specific non-natural amino acid is p-azido-L-phenylalanine. In certain embodiments, the site-specific non-natural amino acid is p-azido-methyl-L-phenylalanine. In certain embodiments, the sites are selected from HC404, HC121, HC180, LC22, LC7, LC42, LC152, HC136, HC25, HC40, HC119, HC190, HC222, HC19, HC52, HC70, HC110, and HC221. In certain embodiments, the sites include HC404. In certain embodiments, the sites include HC180. In certain embodiments, the sites include LC42.

The linker can be any linker deemed suitable by the person of skill. In certain embodiments, the linker is any divalent or multivalent linker known to those of skill in the art. Generally, the linker is capable of forming covalent bonds to the side chain of the glutamine residue of an LQR, X₁-L-Q-G, or L-X₂-Q-G sequence. Useful linkers include primary amine compounds that are suitable substrates for transglutaminase. Exemplary linkers include those described in U.S. Pat. Nos. 9,676,871 and 9,427,478. Useful linkers include lysine (Lys), Ac-Lys-Gly, aminocaproic acid, Ac-Lys-β-Ala, amino-PEG₂(Polyethylene Glycol)-C2, amino-PEG₃-C2, amino-PEG₆-C2, Ac-Lys-Val (valine)-Cit (citrulline)-PABC (p-aminobenzyloxycarbonyl), aminocaproyl-Val-Cit-PABC, putrescine, cadaverine, and Ac-Lys-putrescine, and derivatives thereof.

Additional useful divalent linkers include a bond, alkylene, substituted alkylene, heteroalkylene, substituted heteroalkylene, arylene, substituted arylene, heteroarylene, and substituted heteroarylene. In certain embodiments, the linker is C₁₋₁₀ alkylene or C₁₋₁₀ heteroalkylene. Generally, such linkers comprise a reactive group capable of reacting with a group of the side chain of the receptor amino acid. In certain embodiments, the receptor amino acid comprises an amide, e.g. glutamine, and the linkers comprise an amino group for a transglutaminase reaction with the amide group. In certain embodiments, the receptor amino acid comprises an azide group, and the linkers comprise an alkyne or strained alkyne group for reaction with the azide group.

The molecular payload can be any molecular entity that one of skill in the art might desire to conjugate to the antibody. In certain embodiments, the payload is a therapeutic moiety. In such embodiment, the antibody conjugate can be used to target the therapeutic moiety to its molecular target. In certain embodiments, the payload is a labeling moiety. In such embodiments, the antibody conjugate can be used to detect binding of the antibody to its target. In certain embodiments, the payload is a cytotoxic moiety. In such embodiments, the conjugate can be used target the cytotoxic moiety to a diseased cell, for example a cancer cell, to initiate destruction or elimination of the cell. Conjugates comprising other molecular payloads apparent to those of skill in the art are within the scope of the conjugates described herein.

In certain embodiments, provided herein are conjugates according to Formula (C):

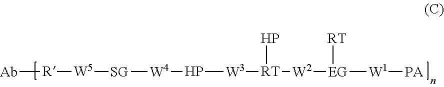

or a pharmaceutically acceptable salt, solvate, stereoisomer, regioisomer, or tautomer thereof, wherein:
Ab is a residue of an antibody or an antigen binding fragment thereof;
PA is a payload moiety;
each W¹, W², W³, W⁴, and W⁵ is independently, at each occurrence, a single bond, absent, or a divalent attaching group;
each EG is independently, at each occurrence, absent, or an eliminator group;
each RT is independently, at each occurrence, a release trigger group, in the backbone of Formula (C) or bonded to EG, wherein each RT is optional;
each HP is independently, at each occurrence, a single bond, absent, a monovalent hydrophilic group, or a divalent hydrophilic group;
each SG is independently, at each occurrence, a single bond, absent, or a divalent spacer group;
each R′ is independently, at each occurrence, a terminal conjugating group, or a divalent residue of a terminal conjugating group;
subscript n is an integer from 1 to 30.

In some embodiments, n is an integer from 1 to 8. In some embodiments, n is 2. In some embodiments, n is 3. In some embodiments, n is 4. In some embodiments, n is 5. In some embodiments, n is 6. In some embodiments, n is 7. In some embodiments, n is 8.

### 2.1 Attaching Groups

Attaching groups facilitate incorporation of eliminator groups, release trigger groups, hydrophobic groups, spacer groups, and/or conjugating groups into a compound. Useful attaching groups are known to, and are apparent to, those of skill in the art. Examples of useful attaching groups are provided herein. In certain embodiments, attaching groups are designated W¹, W², W³, W⁴, or W⁵. In certain embodiments, an attaching group can comprise a divalent ketone, divalent ester, divalent ether, divalent amide, divalent amine, alkylene, arylene, sulfide, disulfide, carbonylene, or a combination thereof. In certain embodiments an attaching group can comprise —C(O)—, —O—, —C(O)NH—, —C(O)NH-alkyl-, —OC(O)NH—, —SC(O)NH—, —NH—, —NH-alkyl-, —C(O)N(CH₃)—, —C(O)N(CH₃)-alkyl-, —N(CH₃)—, —N(CH₃)-alkyl-, —N(CH₃)CH₂CH₂N(CH₃)—, —C(O)CH₂CH₂CH₂C(O)—, —S—, —S—S—, —OCH₂CH₂O—, or the reverse (e.g. —NHC(O)—) thereof, or a combination thereof.

### 2.2 Eliminator Groups

Eliminator groups facilitate separation of a biologically active portion of a compound or conjugate described herein from the remainder of the compound or conjugate in vivo and/or in vitro. Eliminator groups can also facilitate separation of a biologically active portion of a compound or conjugate described herein in conjunction with a release trigger group. For example, the eliminator group and the release trigger group can react in a Releasing Reaction to release a biologically active portion of a compound or conjugate described herein from the compound or conjugate in vivo and/or in vitro. Upon initiation of the Releasing Reaction by the release trigger, the eliminator group cleaves the biologically active moiety, or a prodrug form of the biologically active moiety, and forms a stable, non-toxic entity that has no further effect on the activity of the biologically active moiety.

In certain embodiments, the eliminator group is designated EG herein. Useful eliminator groups include those described herein. In certain embodiments, the eliminator group is:

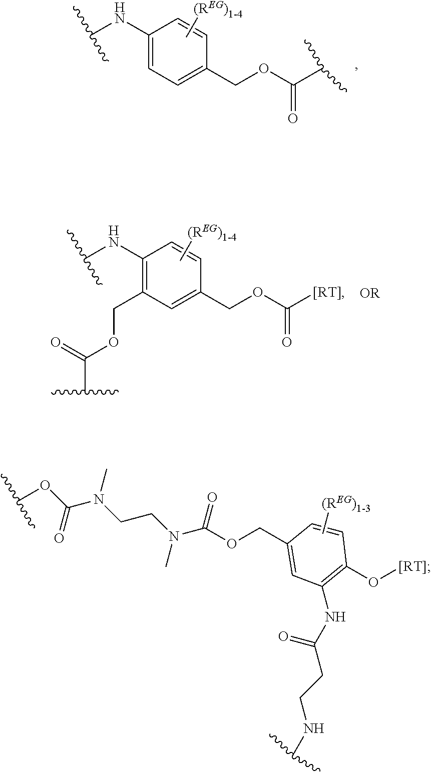

wherein R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the second and third structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In certain embodiments, the eliminator group is

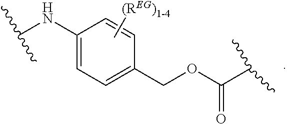

In certain embodiments, the eliminator group is

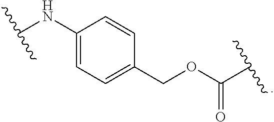 [SMILES: *Nc1ccc(COC(*)=O)cc1]

In certain embodiments, the eliminator group is

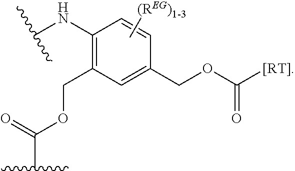

In certain embodiments, the eliminator group is

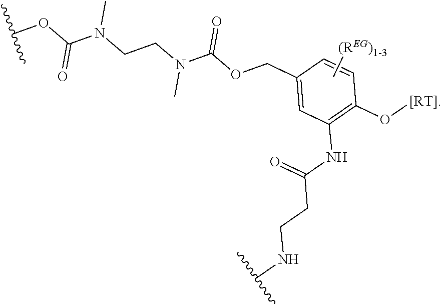

In some embodiments, the eliminator group is:

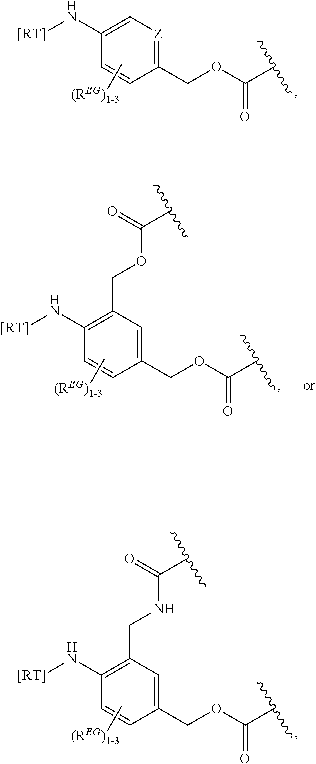

wherein Z may be CH or N, R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the first and second structures, those of skill will recognize that EG is bonded to an RT that is not within the backbone of formula (C1) as indicated in the above description of formula (C1). In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In some embodiments, each R^{EG} in the EG is hydrogen. In certain embodiments, the eliminator group is

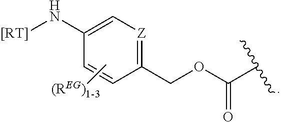

In certain embodiments, the eliminator group is

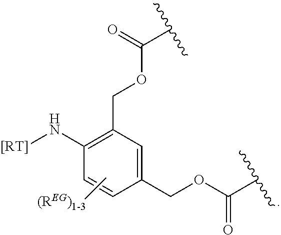

In certain embodiments, the eliminator group is

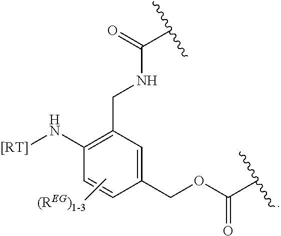

### 2.3 Release Trigger Groups

Release trigger groups facilitate separation of a biologically active portion of a compound or conjugate described herein from the remainder of the compound or conjugate in vivo and/or in vitro. Release trigger groups can also facilitate separation of a biologically active portion of a compound or conjugate described herein in conjunction with an eliminator group. For example, the eliminator group and the release trigger group can react in a Releasing Reaction to release a biologically active portion of a compound or conjugate described herein from the compound or conjugate in vivo and/or in vitro. In certain embodiment, the release trigger can act through a biologically-driven reaction with high tumor:nontumor specificity, such as the proteolytic action of an enzyme overexpressed in a tumor environment.

In certain embodiments, the release trigger group is designated RT herein. In certain embodiments, RT is divalent and bonded within the backbone of formula (C1). In other embodiments, RT is monovalent and bonded to EG as depicted above. Useful release trigger groups include those described herein. In certain embodiments, the release trigger group comprises a residue of a natural or non-natural amino acid or residue of a sugar ring. In certain embodiments, the release trigger group is:

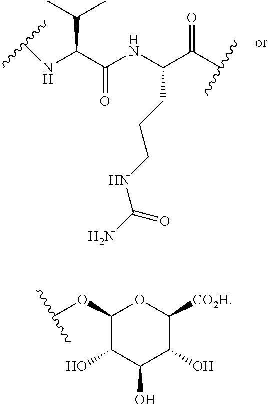 [SMILES: *N[C@H](C(=O)N[C@@H](CCCNC(N)=O)C(*)=O)C(C)C ; *O[C@H]1O[C@@H](C(=O)O)[C@H](O)[C@@H](O)[C@@H]1O]

Those of skill will recognize that the first structure is divalent and can be bonded within the backbone of Formula (C1) or as depicted in Formula (C2), and that the second structure is monovalent and can be bonded to EG as depicted in formula (C1) above.

In certain embodiments, the release trigger group is

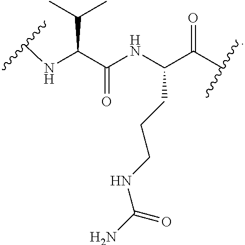 [SMILES: *N[C@H](C(=O)N[C@@H](CCCNC(N)=O)C(*)=O)C(C)C]

In certain embodiments, the release trigger group is

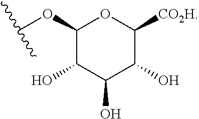 [SMILES: *O[C@H]1O[C@@H](C(=O)O)[C@H](O)[C@@H](O)[C@@H]1O]

In some embodiments, the release trigger group is a protease-cleavable R₁-Val-X₁ peptide having the structure of:

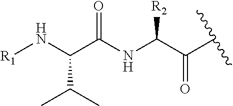

wherein R₁ is a bond to the rest of the compound or

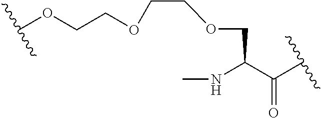 [SMILES: *OCCOCCOC[C@H](NC)C(*)=O]

and R₂ is —CH₃, —CH₂CH₂CO₂H, or —(CH₂)₃NHCONH₂; a legumain-cleavable Ala-Ala-Asn (AAN) or Ala-Ala-Asp (AAD) peptide having the structure of:

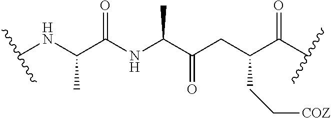

where Z is OH or NH₂; or a β-glucuronidase-cleavable β-glucuronide having the structure of:

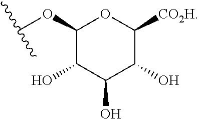 [SMILES: *O[C@H]1O[C@@H](C(=O)O)[C@H](O)[C@@H](O)[C@@H]1O]

Those of skill will recognize that

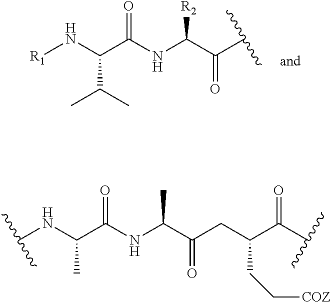

are divalent structures and can be bonded within the backbone of Formula (C1) or as depicted in Formula (C2). The structure

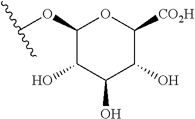 [SMILES: *O[C@H]1O[C@@H](C(=O)O)[C@H](O)[C@@H](O)[C@@H]1O]

is monovalent and can be bonded to EG as depicted in formula (C1) above.

### 2.4 Hydrophilic Groups

Hydrophilic groups facilitate increasing the hydrophilicity of the compounds described herein. It is believed that increased hydrophilicity allows for greater solubility in aqueous solutions, such as aqueous solutions found in biological systems. Hydrophilic groups can also function as spacer groups, which are described in further detail herein.

In certain embodiments, the hydrophilic group is designated HP herein. Useful hydrophilic groups include those described herein. In certain embodiments, the hydrophilic group is a divalent poly(ethylene glycol). In certain embodiments, the hydrophilic group is a divalent poly(ethylene glycol) according to the formula:

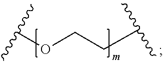

wherein m is an integer from 1 to 13, optionally 1 to 4, optionally 2 to 4, or optionally 4 to 8.

In some embodiments, the hydrophilic group is a divalent poly(ethylene glycol) having the following formula:

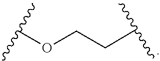 [SMILES: *CCO*]

In some other embodiments, the hydrophilic group is a divalent poly(ethylene glycol) having the following formula:

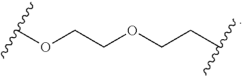 [SMILES: *CCOCCO*]

In other embodiments, the hydrophilic group is a divalent poly(ethylene glycol) having the following formula:

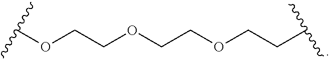 [SMILES: *CCOCCOCCO*]

In other embodiments, the hydrophilic group is a divalent poly(ethylene glycol) having the following formula:

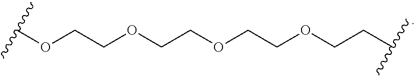 [SMILES: *CCOCCOCCOCCO*]

In some embodiments, the hydrophilic group can bear a chain-presented sulfonic acid having the formula:

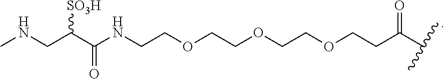 [SMILES: *C(=O)CCOCCOCCOCCNC(=O)C(CNC)S(=O)(=O)O]

### 2.5 Spacer Groups

Spacer groups facilitate spacing of the conjugating group from the other groups of the compounds described herein. This spacing can lead to more efficient conjugation of the compounds described herein to a second compound as well as more efficient cleavage of the active catabolite. The spacer group can also stabilize the conjugating group and lead to improved overall antibody-drug conjugate properties.

In certain embodiments, the spacer group is designated SG herein. Useful spacer groups include those described herein. In certain embodiments, the spacer group is:

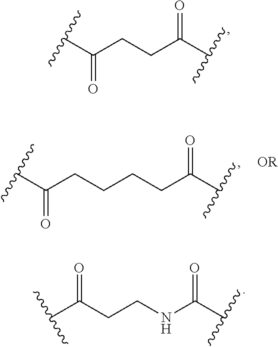 [SMILES: *C(=O)CCC(*)=O ; *C(=O)CCCCC(*)=O ; *C(=O)CCNC(*)=O]

In certain embodiments, the spacer group, W⁴, and the hydrophilic group combine to form a divalent poly(ethylene glycol) according to the formula:

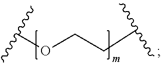

wherein m is an integer from 1 to 13, optionally 1 to 4, optionally 2 to 4, or optionally 4 to 8.

In some embodiments, the SG is

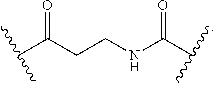 [SMILES: *C(=O)CCNC(*)=O]

In some embodiments, the divalent poly(ethylene glycol) has the following formula:

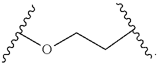 [SMILES: *CCO*]

In some other embodiments, the divalent poly(ethylene glycol) has the following formula:

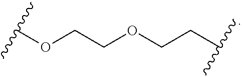 [SMILES: *CCOCCO*]

In other embodiments, the divalent poly(ethylene glycol) has the following formula:

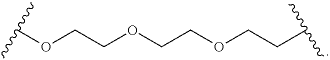 [SMILES: *CCOCCOCCO*]

In other embodiments, the divalent poly(ethylene glycol) has the following formula:

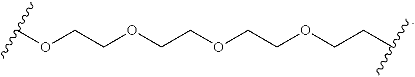 [SMILES: *CCOCCOCCOCCO*]

In some embodiments, the hydrophilic group can bear a chain-presented sulfonic acid having the formula:

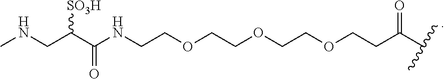 [SMILES: *C(=O)CCOCCOCCOCCNC(=O)C(CNC)S(=O)(=O)O]

### 2.6 Conjugating Groups and Residues Thereof

Conjugating groups facilitate conjugation of the payloads described herein to a second compound, such as an antibody described herein. In certain embodiments, the conjugating group is designated R herein. Conjugating groups can react via any suitable reaction mechanism known to those of skill in the art. In certain embodiments, for conjugating to a receptor glutamine residue, the conjugating group is a primary amine. Those of skill will recognize that a primary amine is capable of forming a bond to a glutamine side chain in a reaction catalyzed by transglutaminase.

In certain embodiments, an additional conjugating group reacts through a [3+2]alkyne-azide cycloaddition reaction, inverse-electron demand Diels-Alder ligation reaction, thiol-electrophile reaction, or carbonyl-oxyamine reaction, as described in detail herein. In certain embodiments, the conjugating group comprises an alkyne, strained alkyne, tetrazine, thiol, para-acetyl-phenylalanine residue, oxyamine, maleimide, or azide. In certain embodiments, the conjugating group is:

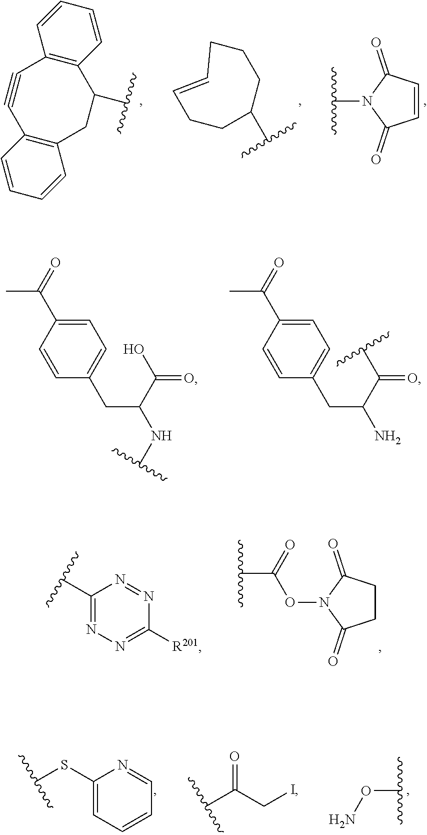

—N₃, or —SH; wherein R²⁰¹ is lower alkyl. In an embodiment, R²⁰¹ is methyl, ethyl, or propyl. In an embodiment, R²⁰¹ is methyl. Additional conjugating groups are described in, for example, U.S. Patent Publication No. 2014/0356385, U.S. Patent Publication No. 2013/0189287, U.S. Patent Publication No. 2013/0251783, U.S. Pat. Nos. 8,703,936, 9,145,361, 9,222,940, and 8,431,558.

After conjugation, a divalent residue of the conjugating group is formed and is bonded to the residue of a second compound. The structure of the divalent residue is determined by the type of conjugation reaction employed to form the conjugate. For receptor glutamine residues, the divalent residue is typically an amide linkage.

In certain embodiments when a conjugate is formed through a [3+2] alkyne-azide cycloaddition reaction, the divalent residue of the conjugating group comprises a triazole ring or fused cyclic group comprising a triazole ring. In certain embodiment when a conjugate is formed through a strain-promoted [3+2] alkyne-azide cycloaddition (SPAAC) reaction, the divalent residue of the conjugating group is:

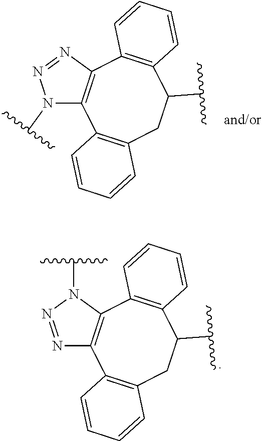

In certain embodiments when a conjugate is formed through a tetrazine inverse electron demand Diels-Alder ligation reaction, the divalent residue of the conjugating group comprises a fused bicyclic ring having at least two adjacent nitrogen atoms in the ring. In certain embodiments when a conjugate is formed through a tetrazine inverse electron demand Diels-Alder ligation reaction, the divalent residue of the conjugating group is:

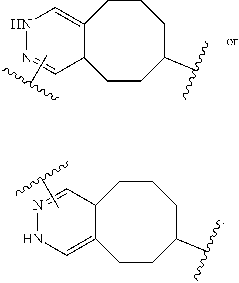

In certain embodiments when a conjugate is formed through a thiol-maleimide reaction, the divalent residue of the conjugating group comprises succinimidylene and a sulfur linkage. In certain embodiments when a conjugate is formed through a thiol-maleimide reaction, the divalent residue of the conjugating group is:

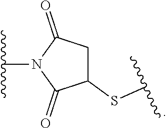 [SMILES: *SC1CC(=O)N(*)C1=O]

In certain embodiments, a conjugate is formed through a thiol-N-hydroxysuccinimide reaction using the following group:

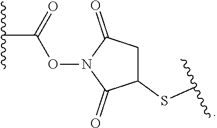 [SMILES: *SC1CC(=O)N(OC(*)=O)C1=O]

The reaction involved for formation of the conjugate comprises the following step:

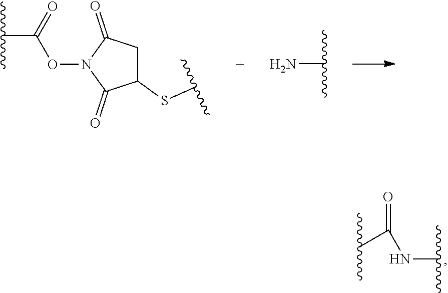

and the resulting divalent residue of the conjugating group is:

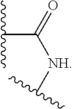 [SMILES: *NC(*)=O]

In certain embodiments when a conjugate is formed through a carbonyl-oxyamine reaction, the divalent residue of the conjugating group comprises a divalent residue of a non-natural amino acid. In certain embodiments when a conjugate is formed through a carbonyl-oxyamine reaction, the divalent residue of the conjugating group is:

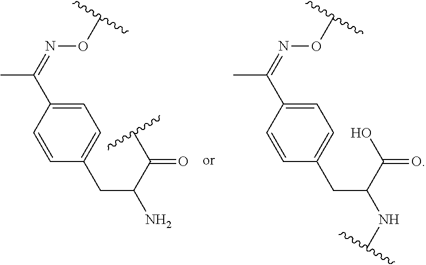 [SMILES: *O/N=C(/C)c1ccc(CC(N)C(*)=O)cc1 ; *NC(Cc1ccc(/C(C)=N\O*)cc1)C(=O)O]

In certain embodiments when a conjugate is formed through a carbonyl-oxyamine reaction, the divalent residue of the conjugating group comprises an oxime linkage. In certain embodiments when a conjugate is formed through a carbonyl-oxyamine reaction, the divalent residue of the conjugating group is:

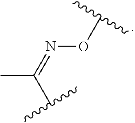 [SMILES: *O/N=C(\*)C]

In some embodiment, provided herein is a conjugate according to Formula (C) or a pharmaceutically acceptable salt, solvate, stereoisomer, or tautomer thereof; wherein EG comprises phenylene, carboxylene, amine, or a combination thereof. In an embodiment, provided herein is a conjugate according to Formula (C), or a pharmaceutically acceptable salt, solvate, stereoisomer, or tautomer thereof; wherein EG is:

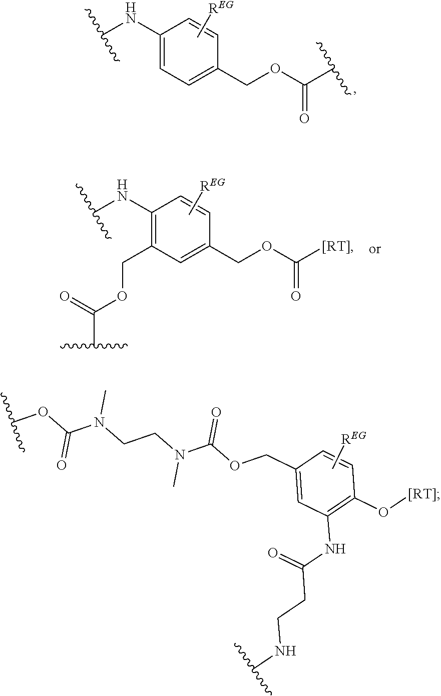

wherein R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the second and third structures, those of skill will recognize that EG is bonded to an RT that is not within the backbone of Formula C1 as indicated in the above description of Formula C1. In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro.

In some embodiments, provided herein is a conjugate according to Formula (C) or a pharmaceutically acceptable salt, solvate, stereoisomer, or tautomer thereof; wherein EG comprises phenylene, carboxylene, amine, or a combination thereof. In an embodiment, provided herein is a conjugate according to Formula (C), or a pharmaceutically acceptable salt, solvate, stereoisomer, or tautomer thereof; wherein EG is:

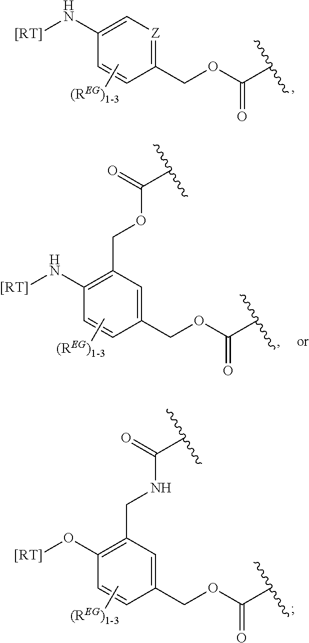

wherein Z may be CH or N, R^{EG} is selected from the group consisting of hydrogen, alkyl, biphenyl, —CF₃, —NO₂, —CN, fluoro, bromo, chloro, alkoxyl, alkylamino, dialkylamino, alkyl-C(O)O—, alkylamino-C(O)— and dialkylaminoC(O)—. In each structure, the phenyl ring can be bound to one, two, three, or in some cases, four R^{EG} groups. In the second and third structures, those of skill will recognize that EG is bonded to an RT that is not within the backbone of Formula C1 as indicated in the above description of Formula C1. In some embodiments, R^{EG} is selected from the group consisting of hydrogen, alkyl, biphenyl, —CF₃, alkoxyl, alkylamino, dialkylamino, alkyl-C(O)O—, alkylamino-C(O)— and dialkylaminoC(O)—. In further embodiments, R^{EG} is selected from the group consisting of hydrogen, —NO₂, —CN, fluoro, bromo, and chloro. In some embodiments, each R^{EG} in the EG is hydrogen.

In some embodiments, provided herein is a conjugate according to Formula (C), or a pharmaceutically acceptable salt, solvate, stereoisomer, or tautomer thereof; wherein RT comprises a residue of a natural or non-natural amino acid or a residue of a sugar. In an embodiment, provided herein is a conjugate according to Formula (C), or a pharmaceutically acceptable salt, solvate, stereoisomer, or tautomer thereof; wherein RT is:

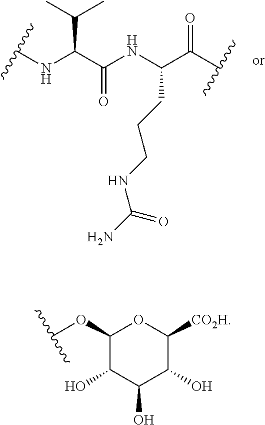 [SMILES: *N[C@H](C(=O)N[C@@H](CCCNC(N)=O)C(*)=O)C(C)C ; *O[C@H]1O[C@@H](C(=O)O)[C@H](O)[C@@H](O)[C@@H]1O]

Those of skill will recognize that the first structure is divalent and can be bonded within the backbone as depicted in Formula (C2), and that the second structure is monovalent and can be bonded to EG as depicted in Formula (C1) above.

In some embodiments, provided herein is a conjugate according to Formula (C), or a pharmaceutically acceptable salt, solvate, stereoisomer, or tautomer thereof; wherein RT comprises a residue of a natural or non-natural amino acid or a residue of a sugar. In an embodiment, provided herein is a conjugate according to Formula (C), or a pharmaceutically acceptable salt, solvate, stereoisomer, or tautomer thereof; wherein RT is:

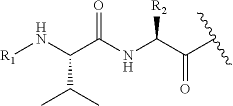

wherein R₁ is a bond to the rest of the compound or

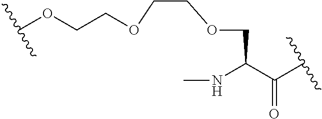 [SMILES: *OCCOCCOC[C@H](NC)C(*)=O]

and R₂ is —CH₃, —CH₂CH₂CO₂H, or —(CH₂)₃NHCONH₂; a legumain-cleavable Ala-Ala-Asn or Ala-Ala-Asp peptide having the structure of:

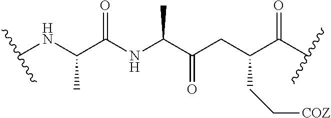

where Z is OH or NH₂; or a β-glucuronidase-cleavable β-glucuronide having the structure of:

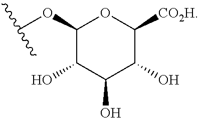 [SMILES: *O[C@H]1O[C@@H](C(=O)O)[C@H](O)[C@@H](O)[C@@H]1O]

Those of skill will recognize that

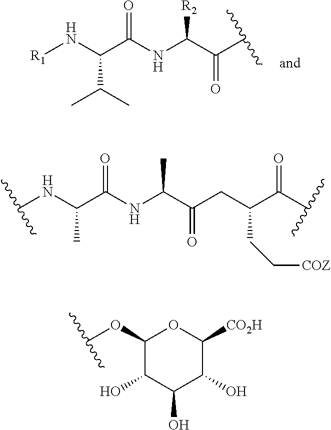

are divalent structures and can be bonded within the backbone of Formula (C1) or as depicted in Formula (C2). The structure is monovalent and can be bonded to EG as depicted in formula (C1) above.

In an embodiment, provided herein is a conjugate according to Formula (C), or a pharmaceutically acceptable salt, solvate, stereoisomer, or tautomer thereof; wherein HP comprises poly(ethylene glycol). In an embodiment, provided herein is a conjugate according to Formula (C), or a pharmaceutically acceptable salt, solvate, stereoisomer, or tautomer thereof, wherein HP is:

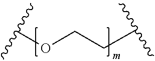

wherein m is an integer from 1 to 13.

In an embodiment, provided herein is a conjugate according to Formula (C), or a pharmaceutically acceptable salt, solvate, stereoisomer, or tautomer thereof; wherein SG comprises C₁-C₁₀ alkylene, C₄-C₆ alkylene, carbonylene, or combination thereof. In an embodiment, provided herein is a conjugate according to Formula (C), or a pharmaceutically acceptable salt, solvate, stereoisomer, or tautomer thereof, wherein SG is:

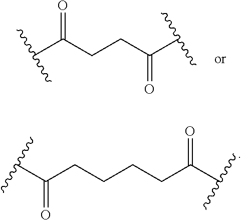 [SMILES: *C(=O)CCC(*)=O ; *C(=O)CCCCC(*)=O]

In an embodiment, provided herein is a conjugate according to Formula (C), or a pharmaceutically acceptable salt, solvate, stereoisomer, or tautomer thereof; wherein W¹, W², W³, W⁴, and W⁵ are each independently a single bond, absent, or comprise a divalent ketone, divalent ester, divalent ether, divalent amide, divalent amine, alkylene, arylene, sulfide, disulfide, carbonylene, or a combination thereof. In an embodiment, provided herein is a conjugate according to Formula (C), or a pharmaceutically acceptable salt, solvate, stereoisomer, or tautomer thereof; wherein W¹, W², W³, W⁴, and W⁵ are each independently a single bond, absent, or comprise —C(O)—, —O—, —C(O)NH—, —C(O)NH-alkyl-, —OC(O)NH—, —SC(O)NH—, —NH—, —NH-alkyl-, —C(O)N(CH₃)—, —C(O)N(CH₃)-alkyl-, —N(CH₃)—, —N(CH₃)-alkyl-, —N(CH₃)CH₂CH₂N(CH₃)—, —C(O)CH₂CH₂CH₂C(O)—, —S—, —S—S—, —OCH₂CH₂O—, or the reverse (e.g. —NHC(O)—) thereof, or a combination thereof.

In an embodiment, provided herein is a conjugate according to Formula (C), or a pharmaceutically acceptable salt, solvate, stereoisomer, or tautomer thereof; wherein R′ comprises a triazole ring. In an embodiment, provided herein is a conjugate according to Formula (C), or a pharmaceutically acceptable salt, solvate, stereoisomer, or tautomer thereof, wherein R′ is a triazole ring or fused cyclic group comprising a triazole ring. In an embodiment, provided herein is a conjugate according to Formula (C), or a pharmaceutically acceptable salt, solvate, stereoisomer, or tautomer thereof; wherein R′ is:

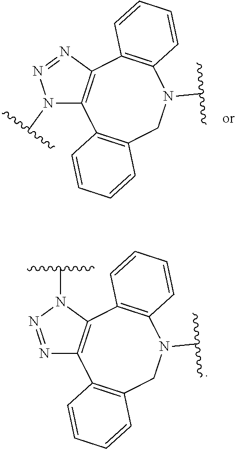

In an embodiment, provided herein is a conjugate according to Formula (C), or a pharmaceutically acceptable salt, solvate, stereoisomer, or tautomer thereof; wherein R′ comprises a fused bicyclic ring having at least two adjacent nitrogen atoms in the ring. In an embodiment, provided herein is a conjugate according to Formula (C), or a pharmaceutically acceptable salt, solvate, stereoisomer, or tautomer thereof, wherein R′ is:

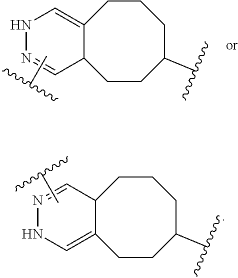

In an embodiment, provided herein is a conjugate according to Formula (C), or a pharmaceutically acceptable salt, solvate, stereoisomer, or tautomer thereof; wherein R′ comprises a sulfur linkage. In an embodiment, provided herein is a conjugate according to Formula (C), or a pharmaceutically acceptable salt, solvate, stereoisomer, or tautomer thereof, wherein R′ is:

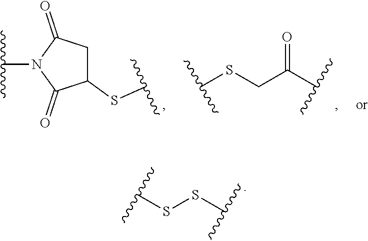

In an embodiment, provided herein is a conjugate according to Formula (C), or a pharmaceutically acceptable salt, solvate, stereoisomer, or tautomer thereof; wherein R′ comprises a divalent residue of a non-natural amino acid. In an embodiment, provided herein is a conjugate according to Formula (C), or a pharmaceutically acceptable salt, solvate, stereoisomer, or tautomer thereof; wherein R′ is:

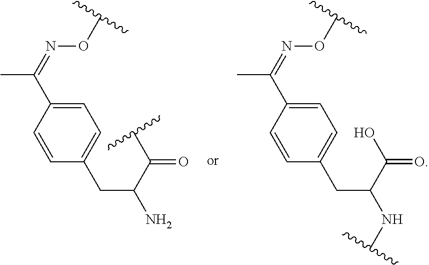 [SMILES: *O/N=C(/C)c1ccc(CC(N)C(*)=O)cc1 ; "or" ; *NC(Cc1ccc(/C(C)=N\O*)cc1)C(=O)O]

In an embodiment, provided herein is a conjugate according to Formula (C), or a pharmaceutically acceptable salt, solvate, stereoisomer, or tautomer thereof; wherein comprises an oxime linkage. In an embodiment, provided herein is a conjugate according to Formula (C), or a pharmaceutically acceptable salt, solvate, stereoisomer, or tautomer thereof, wherein R′ is:

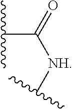 [SMILES: *NC(*)=O]

In an embodiment, provided herein is a conjugate according to Formula (C), or a pharmaceutically acceptable salt, solvate, stereoisomer, or tautomer thereof; wherein comprises an oxime linkage. In an embodiment, provided herein is a conjugate according to Formula (C), or a pharmaceutically acceptable salt, solvate, stereoisomer, or tautomer thereof, wherein R′ is:

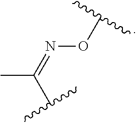 [SMILES: *O/N=C(\*)C]

In an embodiment, provided herein is a conjugate according to Formula (C), or a pharmaceutically acceptable salt, solvate, stereoisomer, or tautomer thereof; wherein R′ is:

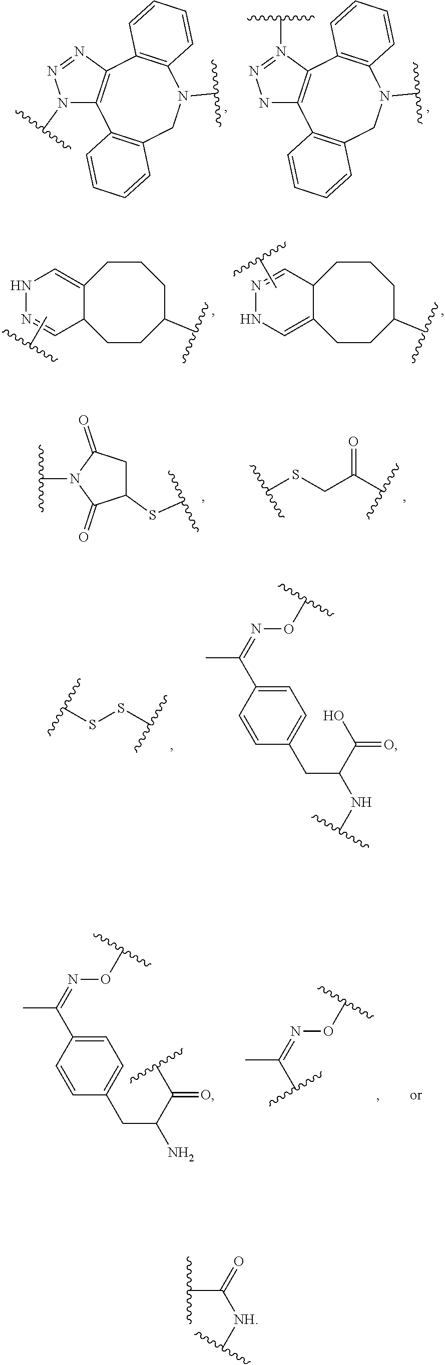

In an embodiment, provided herein is a compound according to Formula (C), or a pharmaceutically acceptable salt, solvate, stereoisomer, or tautomer thereof; wherein Ab is a residue of any compound known to be useful for conjugation to a payload, described herein, and an optional linker, described herein. In an embodiment, provided herein is a compound according to Formula (C), or a pharmaceutically acceptable salt, solvate, stereoisomer, or tautomer thereof, wherein Ab is a residue of an antibody chain, or an antigen binding fragment thereof.

In an aspect, provided herein is an antibody conjugate comprising payloads, described herein, and optional linkers, described herein, linked to an antibody, wherein Ab is a residue of the antibody. In an embodiment, provided herein is an antibody conjugate according to Formula (C), or a pharmaceutically acceptable salt, solvate, stereoisomer, or tautomer thereof, wherein: Ab is a residue of the antibody; and R′ comprises an amide group. Additional payloads may be conjugated to Ab via other reactive groups. In an embodiment, provided herein is an antibody conjugate according to Formula (C), or a pharmaceutically acceptable salt, solvate, stereoisomer, or tautomer thereof, wherein: Ab is a residue of the antibody; and R′ comprises a triazole ring or fused cyclic group comprising a triazole ring. In an embodiment, provided herein is an antibody conjugate according to Formula (C), or a pharmaceutically acceptable salt, solvate, stereoisomer, or tautomer thereof, wherein: Ab is a residue of the antibody; and R′ is:

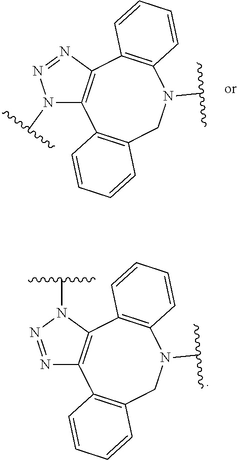 [SMILES: *N1Cc2ccccc2-c2c(nnn2*)-c2ccccc21 ; *N1Cc2ccccc2-c2nnn(*)c2-c2ccccc21]

In an embodiment, provided herein is an antibody conjugate according to Formula (C), or a pharmaceutically acceptable salt, solvate, stereoisomer, or tautomer thereof, wherein: Ab is a residue of the antibody or an antigen binding fragment thereof; and R′ comprises a fused bicyclic ring, wherein the fused bicyclic ring has at least two adjacent nitrogen atoms in the ring. In an embodiment, provided herein is an antibody conjugate according to Formula (C), or a pharmaceutically acceptable salt, solvate, stereoisomer, or tautomer thereof, wherein: Ab is a residue of the antibody or an antigen binding fragment thereof; and R′ is:

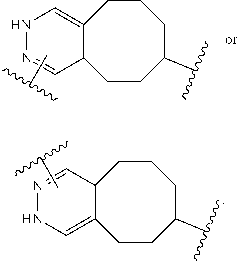

In an embodiment, provided herein is an antibody conjugate according to Formula (C), or a pharmaceutically acceptable salt, solvate, stereoisomer, or tautomer thereof, wherein: Ab is a residue of the polypeptide; and R′ comprises a sulfur linkage. In an embodiment, provided herein is an antibody conjugate according to Formula (C), or a pharmaceutically acceptable salt, solvate, stereoisomer, or tautomer thereof, wherein: Ab is a residue of the polypeptide; and R′ is:

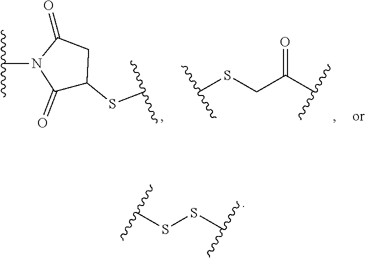

In an embodiment, provided herein is an antibody conjugate according to Formula (C), or a pharmaceutically acceptable salt, solvate, stereoisomer, or tautomer thereof, wherein: Ab is a residue of the polypeptide; and R′ comprises a divalent residue of a non-natural amino acid. In an embodiment, provided herein is an antibody conjugate according to Formula (C), or a pharmaceutically acceptable salt, solvate, stereoisomer, or tautomer thereof, wherein: Ab is a residue of the polypeptide; and R′ is:

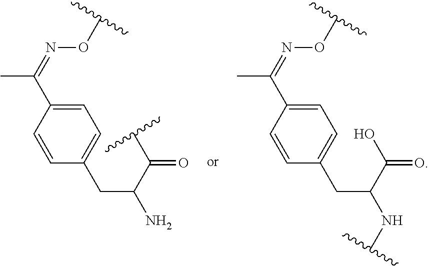 [SMILES: *O/N=C(/C)c1ccc(CC(N)C(*)=O)cc1 ; "or" ; *NC(Cc1ccc(/C(C)=N\O*)cc1)C(=O)O]

In an embodiment, provided herein is an antibody conjugate according to Formula (C), or a pharmaceutically acceptable salt, solvate, stereoisomer, or tautomer thereof, wherein: Ab is a residue of the polypeptide; and R′ comprises an oxime linkage. In an embodiment, provided herein is an antibody conjugate according to Formula (C), or a pharmaceutically acceptable salt, solvate, stereoisomer, or tautomer thereof, wherein: Ab is a residue of the polypeptide; and R′ is:

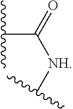 [SMILES: *NC(*)=O]

In an embodiment, provided herein is an antibody conjugate according to Formula (C), or a pharmaceutically acceptable salt, solvate, stereoisomer, or tautomer thereof, wherein: Ab is a residue of the polypeptide; and R′ comprises an oxime linkage. In an embodiment, provided herein is an antibody conjugate according to Formula (C), or a pharmaceutically acceptable salt, solvate, stereoisomer, or tautomer thereof, wherein: Ab is a residue of the polypeptide; and R′ is:

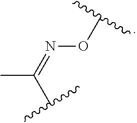 [SMILES: *O/N=C(\*)C]

A linker linked to a drug compound (e.g., cytotoxic drug) is L-PA herein. Subsequently, a L-PA is conjugated to an antibody construct, such as an antibody or an antibody chain, or an antigen binding fragment thereof, to form an antibody conjugate to PA. A linker linked to an immune-modulatory compound is L-IM herein. A L-IM is conjugated to an antibody construct, such as an antibody or an antibody chain, or a an antigen binding fragment thereof, to form an antibody conjugate to IM.

In an embodiment, provided herein is a conjugate according to any of Formulas 106-107, where Ab indicates a residue of the antibody and PA indicates a payload moiety.

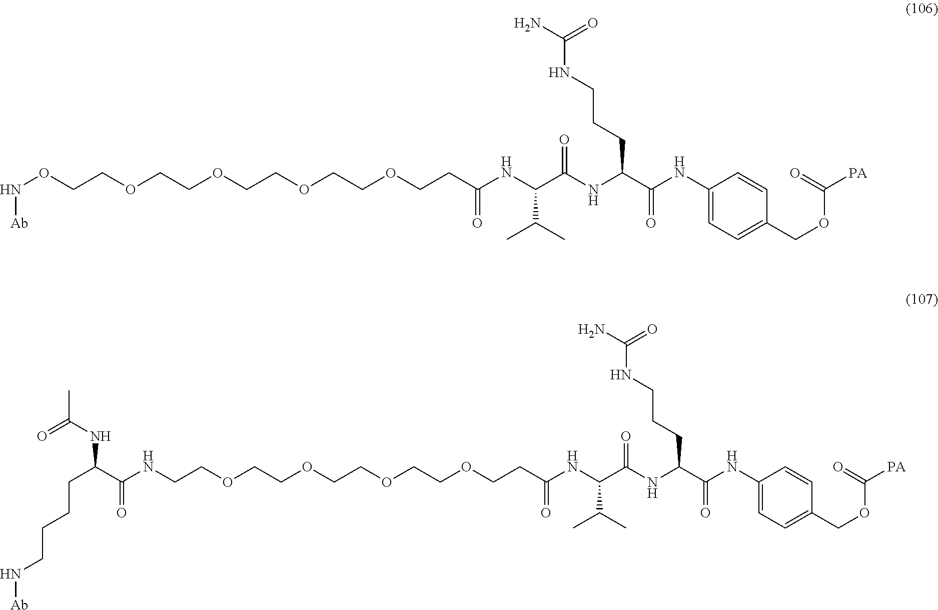

In any of the foregoing embodiments, the conjugate comprises n number of PA moieties, wherein n is an integer from 1 to 8. In some embodiments, n is 2. In some embodiments, n is 3. In some embodiments, n is 4. In some embodiments, n is 5. In some embodiments, n is 6. In some embodiments, n is 7. In some embodiments, n is 8.

In certain embodiments, a conjugate can have a payload selected from the group consisting of a label, a dye, a polymer, a water-soluble polymer, polyethylene glycol, a derivative of polyethylene glycol, a photocrosslinker, a cytotoxic compound, a radionuclide, a drug, an immunostimulatory compound, an affinity label, a photoaffinity label, a reactive compound, a resin, a second protein or polypeptide or polypeptide analog, an antibody or antibody fragment, a metal chelator, a cofactor, a fatty acid, a carbohydrate, a polynucleotide, a DNA, a RNA, an antisense polynucleotide, a peptide, a water-soluble dendrimer, a cyclodextrin, an inhibitory ribonucleic acid, a biomaterial, a nanoparticle, a spin label, a fluorophore, a metal-containing moiety, a radioactive moiety, a novel functional group, a group that covalently or noncovalently interacts with other molecules, a photocaged moiety, a photoisomerizable moiety, biotin, a derivative of biotin, a biotin analogue, a moiety incorporating a heavy atom, a chemically cleavable group, a photocleavable group, an elongated side chain, a carbon-linked sugar, a redox-active agent, an amino thioacid, a toxic moiety, an isotopically labeled moiety, a biophysical probe, a phosphorescent group, a chemiluminescent group, an electron dense group, a magnetic group, an intercalating group, a chromophore, an energy transfer agent, a biologically active agent, a detectable label, a small molecule, or any combination thereof.

Useful drug payloads include any cytotoxic, cytostatic or immunomodulatory drug. Useful classes of cytotoxic or immunomodulatory agents include, for example, antitubulin agents, auristatins, DNA minor groove binders, DNA replication inhibitors, alkylating agents (e.g., platinum complexes such as cis-platin, mono(platinum), bis(platinum) and tri-nuclear platinum complexes and carboplatin), anthracyclines, antibiotics, antifolates, antimetabolites, calmodulin inhibitors, chemotherapy sensitizers, duocarmycins, etoposides, fluorinated pyrimidines, ionophores, lexitropsins, maytansinoids, nitrosoureas, platinols, pore-forming compounds, purine antimetabolites, puromycins, radiation sensitizers, rapamycins, steroids, taxanes, topoisomerase inhibitors, vinca alkaloids, or the like.

Individual cytotoxic or immunomodulatory agents include, for example, an androgen, anthramycin (AMC), asparaginase, 5-azacytidine, azathioprine, bleomycin, busulfan, buthionine sulfoximine, calicheamicin, calicheamicin derivatives, camptothecin, carboplatin, carmustine (BSNU), CC-1065, chlorambucil, cisplatin, colchicine, cyclophosphamide, cytarabine, cytidine arabinoside, cytochalasin B, dacarbazine, dactinomycin (formerly actinomycin), daunorubicin, decarbazine, DM1, DM4, docetaxel, doxorubicin, etoposide, an estrogen, 5-fluordeoxyuridine, 5-fluorouracil, gemcitabine, gramicidin D, hydroxyurea, idarubicin, ifosfamide, irinotecan, lomustine (CCNU), maytansine, mechlorethamine, melphalan, 6-mercaptopurine, methotrexate, mithramycin, mitomycin C, mitoxantrone, nitroimidazole, paclitaxel, palytoxin, plicamycin, procarbizine, rhizoxin, streptozotocin, tenoposide, 6-thioguanine, thioTEPA, topotecan, vinblastine, vincristine, vinorelbine, VP-16 and VM-26.

In some embodiments, suitable cytotoxic agents include, for example, DNA minor groove binders (e.g., enediynes and lexitropsins, a CBI compound; see also U.S. Pat. No. 6,130,237), duocarmycins, taxanes (e.g., paclitaxel and docetaxel), puromycins, vinca alkaloids, CC-1065, SN-38, topotecan, morpholino-doxorubicin, rhizoxin, cyanomorpholino-doxorubicin, echinomycin, combretastatin, netropsin, epothilone A and B, estramustine, cryptophycins, cemadotin, maytansinoids, discodermolide, eleutherobin, and mitoxantrone.

In some embodiments, the payload is an anti-tubulin agent. Examples of anti-tubulin agents include, but are not limited to, taxanes (e.g., Taxol® (paclitaxel), Taxotere® (docetaxel)), T67 (Tularik) and vinca alkyloids (e.g., vincristine, vinblastine, vindesine, and vinorelbine). Other antitubulin agents include, for example, baccatin derivatives, taxane analogs, epothilones (e.g., epothilone A and B), nocodazole, colchicine and colcimid, estramustine, cryptophycins, cemadotin, maytansinoids, combretastatins, discodermolide, and eleutherobin.

In certain embodiments, the cytotoxic agent is a maytansinoid, another group of anti-tubulin agents. For example, in specific embodiments, the maytansinoid can be maytansine or DM-1 (ImmunoGen, Inc.; see also Chari et al., 1992, Cancer Res. 52:127-131).

In some embodiments, the payload is an auristatin, such as auristatin E or a derivative thereof. For example, the auristatin E derivative can be an ester formed between auristatin E and a keto acid. For example, auristatin E can be reacted with paraacetyl benzoic acid or benzoylvaleric acid to produce AEB and AEVB, respectively. Other typical auristatin derivatives include AFP, MMAF, and MMAE. The synthesis and structure of auristatin derivatives are described in U.S. Patent Application Publication Nos. 2003-0083263, 2005-0238649 and 2005-0009751; International Patent Publication No. WO 04/010957, International Patent Publication No. WO 02/088172, and U.S. Pat. Nos. 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414.

In some embodiments, the payload is not a radioisotope. In some embodiments, the payload is not radioactive.

In some embodiments, the payload is an antimetabolite. The antimetabolite can be, for example, a purine antagonist (e.g., azothioprine or mycophenolate mofetil), a dihydrofolate reductase inhibitor (e.g., methotrexate), acyclovir, ganciclovir, zidovudine, vidarabine, ribavarin, azidothymidine, cytidine arabinoside, amantadine, dideoxyuridine, iododeoxyuridine, poscarnet, or trifluridine.

In other embodiments, the payload is tacrolimus, cyclosporine, FU506 or rapamycin. In further embodiments, the Drug is aldesleukin, alemtuzumab, alitretinoin, allopurinol, altretamine, amifostine, anastrozole, arsenic trioxide, bexarotene, bexarotene, calusterone, capecitabine, celecoxib, cladribine, Darbepoetin alfa, Denileukin diftitox, dexrazoxane, dromostanolone propionate, epirubicin, Epoetin alfa, estramustine, exemestane, Filgrastim, floxuridine, fludarabine, fulvestrant, gemcitabine, gemtuzumab ozogamicin (MYLOTARG), goserelin, idarubicin, ifosfamide, imatinib mesylate, Interferon alfa-2a, irinotecan, letrozole, leucovorin, levamisole, meclorethamine or nitrogen mustard, megestrol, mesna, methotrexate, methoxsalen, mitomycin C, mitotane, nandrolone phenpropionate, oprelvekin, oxaliplatin, pamidronate, pegademase, pegaspargase, pegfilgrastim, pentostatin, pipobroman, plicamycin, porfimer sodium, procarbazine, quinacrine, rasburicase, Rituximab, Sargramostim, streptozocin, tamoxifen, temozolomide, teniposide, testolactone, thioguanine, toremifene, Tositumomab, Trastuzumab (HERCEPTIN), tretinoin, uracil mustard, valrubicin, vinblastine, vincristine, vinorelbine or zoledronate.

In some embodiments, the payload is an immunomodulatory agent. The immunomodulatory agent can be, for example, ganciclovir, etanercept, tacrolimus, cyclosporine, rapamycin, cyclophosphamide, azathioprine, mycophenolate mofetil or methotrexate. Alternatively, the immunomodulatory agent can be, for example, a glucocorticoid (e.g., cortisol or aldosterone) or a glucocorticoid analogue (e.g., prednisone or dexamethasone).

In some embodiments, the immunomodulatory agent is an anti-inflammatory agent, such as arylcarboxylic derivatives, pyrazole-containing derivatives, oxicam derivatives and nicotinic acid derivatives. Classes of anti-inflammatory agents include, for example, cyclooxygenase inhibitors, 5-lipoxygenase inhibitors, and leukotriene receptor antagonists.

Suitable cyclooxygenase inhibitors include meclofenamic acid, mefenamic acid, carprofen, diclofenac, diflunisal, fenbufen, fenoprofen, indomethacin, ketoprofen, nabumetone, sulindac, tenoxicam and tolmetin.

Suitable lipoxygenase inhibitors include redox inhibitors (e.g., catechol butane derivatives, nordihydroguaiaretic acid (NDGA), masoprocol, phenidone, Ianopalen, indazolinones, naphazatrom, benzofuranol, alkylhydroxylamine), and non-redox inhibitors (e.g., hydroxythiazoles, methoxyalkylthiazoles, benzopyrans and derivatives thereof, methoxytetrahydropyran, boswellic acids and acetylated derivatives of boswellic acids, and quinolinemethoxyphenylacetic acids substituted with cycloalkyl radicals), and precursors of redox inhibitors.

Other suitable lipoxygenase inhibitors include antioxidants (e.g., phenols, propyl gallate, flavonoids and/or naturally occurring substrates containing flavonoids, hydroxylated derivatives of the flavones, flavonol, dihydroquercetin, luteolin, galangin, orobol, derivatives of chalcone, 4,2′,4′-trihydroxychalcone, ortho-aminophenols, N-hydroxyureas, benzofuranols, ebselen and species that increase the activity of the reducing selenoenzymes), iron chelating agents (e.g., hydroxamic acids and derivatives thereof, N-hydroxyureas, 2-benzyl-1-naphthol, catechols, hydroxylamines, camosol trolox C, catechol, naphthol, sulfasalazine, zyleuton, 5-hydroxyanthranilic acid and 4-(omega-arylalkyl)phenylalkanoic acids), imidazole-containing compounds (e.g., ketoconazole and itraconazole), phenothiazines, and benzopyran derivatives.

Yet other suitable lipoxygenase inhibitors include inhibitors of eicosanoids (e.g., octadecatetraenoic, eicosatetraenoic, docosapentaenoic, eicosahexaenoic and docosahexaenoic acids and esters thereof, PGE1 (prostaglandin E1), PGA2 (prostaglandin A2), viprostol, 15-monohydroxyeicosatetraenoic, 15-monohydroxy-eicosatrienoic and 15-monohydroxyeicosapentaenoic acids, and leukotrienes B5, C5 and D5), compounds interfering with calcium flows, phenothiazines, diphenylbutylamines, verapamil, fuscoside, curcumin, chlorogenic acid, caffeic acid, 5,8,11,14-eicosatetrayenoic acid (ETYA), hydroxyphenylretinamide, Ionapalen, esculin, diethylcarbamazine, phenantroline, baicalein, proxicromil, thioethers, diallyl sulfide and di-(1-propenyl) sulfide.

Leukotriene receptor antagonists include calcitriol, ontazolast, Bayer Bay-x-1005, Ciba-Geigy CGS-25019C, ebselen, Leo Denmark ETH-615, Lilly LY-293111, Ono ONO-4057, Terumo TMK-688, Boehringer Ingleheim BI-RM-270, Lilly LY 213024, Lilly LY 264086, Lilly LY 292728, Ono ONO LB457, Pfizer 105696, Perdue Frederick PF 10042, Rhone-Poulenc Rorer RP 66153, SmithKline Beecham SB-201146, SmithKline Beecham SB-201993, SmithKline Beecham SB-209247, Searle SC-53228, Sumitamo SM 15178, American Home Products WAY 121006, Bayer Bay-o-8276, Warner-Lambert CI-987, Warner-Lambert CI-987BPC-15LY 223982, Lilly LY 233569, Lilly LY-255283, MacroNex MNX-160, Merck and Co. MK-591, Merck and Co. MK-886, Ono ONO-LB-448, Purdue Frederick PF-5901, Rhone-Poulenc Rorer RG14893, Rhone-Poulenc Rorer RP 66364, Rhone-Poulenc Rorer RP 69698, Shionoogi S-2474, Searle SC-41930, Searle SC-50505, Searle SC-51146, Searle SC-52798, SmithKline Beecham SK&F-104493, Leo Denmark SR-2566, Tanabe T-757 and Teijin TEI-1338.

Other useful drug payloads include chemical compounds useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, AstraZeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (TYKERB®, GSK572016, Glaxo Smith Kline), Lonafarnib (SCH 66336), Sorafenib (BAY43-9006, Bayer Labs), and Gefitinib (IRESSA®, AstraZeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CBT-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin γll and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamniprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (doxetaxel; Rhone-Poulenc Rorer, Antony, France); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Other useful payloads include: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-α, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN® rIL-2; a topoisomerase 1 inhibitor such as LURTOTECAN®; ABARELIX® rmRH; (ix) anti-angiogenic agents such as bevacizumab (AVASTIN®, Genentech); and (x) pharmaceutically acceptable salts, acids and derivatives of any of the above. Other anti-angiogenic agents include MMP-2 (matrix-metalloproteinase 2) inhibitors, MMP-9 (matrix-metalloproteinase 9) inhibitors, COX-II (cyclooxygenase II) inhibitors, and VEGF receptor tyrosine kinase inhibitors. Examples of such useful matrix metalloproteinase inhibitors that can be used in combination with the present compounds/compositions are described in WO 96/33172, WO 96/27583, EP 818442, EP 1004578, WO 98/07697, WO 98/03516, WO 98/34918, WO 98/34915, WO 98/33768, WO 98/30566, EP 606,046, EP 931,788, WO 90/05719, WO 99/52910, WO 99/52889, WO 99/29667, WO 99/07675, EP 945864, U.S. Pat. Nos. 5,863,949, 5,861,510, and EP 780,386, all of which are incorporated herein in their entireties by reference. Examples of VEGF receptor tyrosine kinase inhibitors include 4-(4-bromo-2-fluoroanilino)-6-methoxy-7-(1-methylpiperidin-4-ylmethoxy)qu- -inazoline (ZD6474; Example 2 within WO 01/32651), 4-(4-fluoro-2-methylindol-5-yloxy)-6-methoxy-7-(3-pyrrolidin-1-ylpropoxy)- -quinazoline (AZD2171; Example 240 within WO 00/47212), vatalanib (PTK787; WO 98/35985) and SU11248 (sunitinib; WO 01/60814), and compounds such as those disclosed in PCT Publication Nos. WO 97/22596, WO 97/30035, WO 97/32856, and WO 98/13354).

In certain embodiments, the payload is an antibody or an antibody fragment. In certain embodiments, the payload antibody or fragment can be encoded by any of the immunoglobulin genes recognized by those of skill in the art. The immunoglobulin genes include, but are not limited to, the κ, λ, α, γ (IgG1, IgG2, IgG3, and IgG4), δ, ε and μ constant region genes, as well as the immunoglobulin variable region genes. The term includes full-length antibodies and antibody fragments recognized by those of skill in the art, and variants thereof. Exemplary fragments include but are not limited to Fv, Fe, Fab, and (Fab′)₂, single chain Fv (scFv), diabodies, triabodies, tetrabodies, bifunctional hybrid antibodies, CDR1, CDR2, CDR3, combinations of CDR's, variable regions, framework regions, constant regions, and the like.

In certain embodiments, the payload is one or more water-soluble polymers. A wide variety of macromolecular polymers and other molecules can be linked to antigen-binding polypeptides of the present invention to modulate biological properties of the antibody, and/or provide new biological properties to the antibody. These macromolecular polymers can be linked to the antibody via a naturally encoded amino acid, via a non-naturally encoded amino acid, or any functional substituent of a natural or non-natural amino acid, or any substituent or functional group added to a natural or non-natural amino acid. The molecular weight of the polymer may be of a wide range, including but not limited to, between about 100 Da and about 100,000 Da or more.

The polymer selected may be water soluble so that the protein to which it is attached does not precipitate in an aqueous environment, such as a physiological environment. The polymer may be branched or unbranched. Preferably, for therapeutic use of the end-product preparation, the polymer will be pharmaceutically acceptable.

The proportion of polyethylene glycol molecules to antibody molecules will vary, as will their concentrations in the reaction mixture. In general, the optimum ratio (in terms of efficiency of reaction in that there is minimal excess unreacted protein or polymer) may be determined by the molecular weight of the polyethylene glycol selected and on the number of available reactive groups available. As relates to molecular weight, typically the higher the molecular weight of the polymer, the fewer number of polymer molecules which may be attached to the protein. Similarly, branching of the polymer should be taken into account when optimizing these parameters. Generally, the higher the molecular weight (or the more branches) the higher the polymer:protein ratio.

The water soluble polymer may be any structural form including but not limited to linear, forked or branched. Typically, the water soluble polymer is a poly(alkylene glycol), such as poly(ethylene glycol) (PEG), but other water soluble polymers can also be employed. By way of example, PEG is used to describe certain embodiments of this invention.

PEG is a well-known, water soluble polymer that is commercially available or can be prepared by ring-opening polymerization of ethylene glycol according to methods well known in the art (Sandler and Karo, Polymer Synthesis, Academic Press, New York, Vol. 3, pages 138-161). The term “PEG” is used broadly to encompass any polyethylene glycol molecule, without regard to size or to modification at an end of the PEG, and can be represented as linked to the antibody by the formula: XO—(CH₂CH₂O)₌ₙ—CH₂CH₂—Y where n is 2 to 10,000 and X is H or a terminal modification, including but not limited to, a C₁₋₄ alkyl.

In some cases, a PEG used in the invention terminates on one end with hydroxy or methoxy, i.e., X is H or CH₃ (“methoxy PEG”). Alternatively, the PEG can terminate with a reactive group, thereby forming a bifunctional polymer. Typical reactive groups can include those reactive groups that are commonly used to react with the functional groups found in the 20 common amino acids (including but not limited to, maleimide groups, activated carbonates (including but not limited to, p-nitrophenyl ester), activated esters (including but not limited to, N-hydroxysuccinimide, p-nitrophenyl ester) and aldehydes) as well as functional groups that are inert to the 20 common amino acids but that react specifically with complementary functional groups present in non-naturally encoded amino acids (including but not limited to, azide groups, alkyne groups). It is noted that the other end of the PEG, which is shown in the above formula by Y, will attach either directly or indirectly to an antigen-binding polypeptide via a naturally-occurring or non-naturally encoded amino acid. For instance, Y may be an amide, carbamate or urea linkage to an amine group (including but not limited to, the epsilon amine of lysine or the N-terminus) of the polypeptide. Alternatively, Y may be a maleimide linkage to a thiol group (including but not limited to, the thiol group of cysteine). Alternatively, Y may be a linkage to a residue not commonly accessible via the 20 common amino acids. For example, an azide group on the PEG can be reacted with an alkyne group on the antibody to form a Huisgen [3+2] cycloaddition product. Alternatively, an alkyne group on the PEG can be reacted with an azide group present in a non-naturally encoded amino acid to form a similar product. A strained alkene group on the PEG can be reacted with a tetrazine group on the antibody to form a tetrazine ligation product. Alternatively, a tetrazine group on the PEG can be reacted with strained alkene group present in a non-naturally encoded amino acid to form a similar product.

Any molecular mass for a PEG can be used as practically desired, including but not limited to, from about 100 Daltons (Da) to 100,000 Da or more as desired (including but not limited to, sometimes 0.1-50 kDa or 10-40 kDa). Branched chain PEGs, including but not limited to, PEG molecules with each chain having a MW ranging from 1-100 kDa (including but not limited to, 1-50 kDa or 5-20 kDa) can also be used. A wide range of PEG molecules are described in, including but not limited to, the Shearwater Polymers, Inc. catalog, Nektar Therapeutics catalog, incorporated herein by reference.

The polymer backbone can be linear or branched. Branched polymer backbones are generally known in the art. Typically, a branched polymer has a central branch core moiety and a plurality of linear polymer chains linked to the central branch core. PEG is commonly used in branched forms that can be prepared by addition of ethylene oxide to various polyols, such as glycerol, glycerol oligomers, pentaerythritol and sorbitol. The central branch moiety can also be derived from several amino acids, such as lysine. The branched poly(ethylene glycol) can be represented in general form as R(-PEG-OH)m in which R is derived from a core moiety, such as glycerol, glycerol oligomers, or pentaerythritol, and m represents the number of arms. Multi-armed PEG molecules, such as those described in U.S. Pat. Nos. 5,932,462 5,643,575; 5,229,490; 4,289,872; U.S. Pat. Appl. 2003/0143596; WO 96/21469; and WO 93/21259, each of which is incorporated by reference herein in its entirety, can also be used as the polymer backbone.

Branched PEG can also be in the form of a forked PEG represented by PEG(-YCHZ₂)ₙ, where Y is a linking group and Z is an activated terminal group linked to CH by a chain of atoms of defined length.

Yet another branched form, the pendant PEG, has reactive groups, such as carboxyl, along the PEG backbone rather than at the end of PEG chains.

In addition to these forms of PEG, the polymer can also be prepared with weak or degradable linkages in the backbone. For example, PEG can be prepared with ester linkages in the polymer backbone that are subject to hydrolysis. As shown below, this hydrolysis results in cleavage of the polymer into fragments of lower molecular weight: -PEG-C02-PEG-+H₂O→PEG-CO₂H+HO-PEG- It is understood by those skilled in the art that the term poly(ethylene glycol) or PEG represents or includes all the forms known in the art including but not limited to those disclosed herein.

Many other polymers are also suitable for use in the present invention. In some embodiments, polymer backbones that are water-soluble, with from 2 to about 300 termini, are particularly useful in the invention. Examples of suitable polymers include, but are not limited to, other poly(alkylene glycols), such as poly(propylene glycol) (“PPG”), copolymers thereof (including but not limited to copolymers of ethylene glycol and propylene glycol), terpolymers thereof, mixtures thereof, and the like. Although the molecular weight of each chain of the polymer backbone can vary, it is typically in the range of from about 800 Da to about 100,000 Da, often from about 6,000 Da to about 80,000 Da.

Those of ordinary skill in the art will recognize that the foregoing list for substantially water soluble backbones is by no means exhaustive and is merely illustrative, and that all polymeric materials having the qualities described above are contemplated as being suitable for use in the present invention.

Conjugation

Generally, conjugation proceeds according to standard techniques known to those of skill in the art.

In certain embodiments, a payload comprising an amino group, or a linker-payload comprising an amino group, is contacted with an antibody comprising a receptor glutamine residue under conditions suitable for a transglutamination reaction at the glutamine side chain. Typically, this is in the presence of a transglutaminase enzyme suitable for catalyzing the reaction. Useful transglutaminase enzymes are commercially available. In certain embodiments, the enzyme is a bacterial transglutaminase. In certain embodiments, the enzyme is ZsmTG, a transglutaminase of Streptomyces mobrensis (Zedria GmbH). In certain embodiments, the enzyme is Streptomyces hydroscopicus transglutaminase.

In most embodiments, standard transglutaminase conditions are used. In certain embodiments, the conditions of the enzyme's supplier are followed. In certain embodiments, 1-60 molar equivalents of antibody are contacted with 1-60 molar equivalents of payload or linker-payload.

In certain embodiments, the concentration of antibody is from about 0.1 mg/ml to about 100 mg/ml, e.g. from about 0.5 mg/ml to about 75 mg/ml, from about 1 mg/ml to about 50 mg/ml, from about 2.5 mg/ml to about 45 mg/ml, from about 5 mg/ml to about 40 mg/ml, from about 10 mg/ml to about 35 mg/ml, from about 12.5 mg/ml to about 30 mg/ml, from about 15 mg/ml to about 25 mg/ml, from about 17.5 to about 20 mg/ml, e.g. 0.15 mg/ml, 0.2 mg/ml, 0.25 mg/ml, 0.3 mg/ml, 0.35 mg/ml, 0.4 mg/ml, 0.45 mg/ml, 0.5 mg/ml, 0.55 mg/mi, 0.6 mg/ml, 0.7 mg/ml, 0.8 mg/ml, 0.9 mg/mi, 2 mg/ml, 3 mg/ml, 4, mg/ml, 6 mg/ml, 7 mg/mi, 8 mg/ml, 9 mg/ml, 11 mg/ml, 12 mg/ml, 13 mg/ml, 14 mg/ml, 16 mg/ml, 19 mg/ml, 23 mg/ml, 27 mg/ml, 31 mg/ml, 33 mg/ml, 37.5 mg/mi, 41 mg/ml, 42 mg/ml, 43 mg/ml, 44 mg/ml, 46 mg/ml, 47 mg/ml, 48 mg/ml, or 49 mg/ml.

In certain embodiments, the concentration of payload or linker-payload is from about 0.1 mg/ml to about 100 mg/ml, e.g. from about 0.5 mg/ml to about 75 mg/ml, from about 1 mg/ml to about 50 mg/ml, from about 2.5 mg/ml to about 45 mg/ml, from about 5 mg/ml to about 40 mg/ml, from about 10 mg/ml to about 35 mg/ml, from about 12.5 mg/ml to about 30 mg/ml, from about 15 mg/ml to about 25 mg/ml, from about 17.5 to about 20 mg/ml, e.g. 0.15 mg/ml, 0.2 mg/ml, 0.25 mg/ml, 0.3 mg/ml, 0.35 mg/ml, 0.4 mg/ml, 0.45 mg/ml, 0.5 mg/ml, 0.55 mg/mi, 0.6 mg/ml, 0.7 mg/ml, 0.8 mg/ml, 0.9 mg/mi, 2 mg/ml, 3 mg/ml, 4, mg/ml, 6 mg/ml, 7 mg/mi, 8 mg/ml, 9 mg/ml, 11 mg/ml, 12 mg/ml, 13 mg/ml, 14 mg/ml, 16 mg/ml, 19 mg/ml, 23 mg/ml, 27 mg/ml, 31 mg/ml, 33 mg/ml, 37.5 mg/mi, 41 mg/ml, 42 mg/ml, 43 mg/ml, 44 mg/ml, 46 mg/ml, 47 mg/ml, 48 mg/ml, or 49 mg/ml.

In certain embodiments, the transglutaminase is present in an amount from about 0.01 mol equivalents to about 2 mol equivalents to one or the other substrate, from about 0.05 mol equivalents to about 1.5 mol equivalents, from about 0.1 mol equivalents to about 1.125 mol equivalents, from about 0.125 mol equivalents to about 1.75 mol equivalents, from about 0.25 mol equivalents, 0.3 mol equivalents, 0.4 mol equivalents, 0.5 mol equivalents, 0.6 mol equivalents, 0.7 mol equivalents, 0.8 mol equivalents to about 1 mol equivalents, or from about 1 mol equivalents to about 2.5 mol equivalents.

In certain embodiments, the reaction proceeds at a suitable temperature. In certain embodiments, the reaction proceeds at 25° C.-37° C. In certain embodiments, the reaction proceeds for a suitable time. In certain embodiments, the reaction proceeds for about 1-5 hours, or for about 2-4 hours, or for about 2.5 hours to about 3.5 h, or for about 1 hour, 2 hours, 3 hours, 4 hours.

The reaction proceeds at a suitable pH. In certain embodiments, the reaction proceeds at a pH of 6.0-8.0, for instance at about pH 6.5, at about pH 7.0, at about pH 7.5, or at about pH 8.0. The pH buffer is any buffer deemed suitable. In certain embodiments, the pH buffer is HEPES (4-2-hydroxyethyl-1-piperazineethanesuifonic acid), MOPS (3-(N-morpholino)propanesulfonic acid), or PIPES (piperazine-N,N-bis(2-ethanesulfonic acid) at a pH of about pH7.0.

For conjugation to site-specific non-natural amino acids, standard techniques are used. For instance, conjugation to an azide-bearing non-natural amino acid is described extensively in U.S. Pat. Nos. 9,682,934, 9,738,724, and Kline et al., 2015, Pharm. Res. 32:3480-3493, the contents of which are hereby incorporated by reference in their entireties.

Parent Antibodies

The parent antibody can be any antibody known to those of skill in the art, or later discovered, without limitation. The parent antibody may be substantially encoded by an antibody gene or antibody genes from any organism, including but not limited to humans, mice, rats, rabbits, camels, llamas, dromedaries, monkeys, particularly mammals and particularly human and particularly mice and rats. In one embodiment, the parent antibody may be fully human, obtained for example from a patient or subject, by using transgenic mice or other animals (Bruggemann & Taussig, 1997, Curr. Opin. Biotechnol. 8:455-458) or human antibody libraries coupled with selection methods (Griffiths & Duncan, 1998, Curr. Opin. Biotechnol. 9:102-108). The parent antibody may be from any source, including artificial or naturally occurring. For example parent antibody can be an engineered antibody, including but not limited to chimeric antibodies and humanized antibodies (Clark, 2000, Immunol. Today 21:397-402) or derived from a combinatorial library. In addition, the parent antibody may be an engineered variant of an antibody that is substantially encoded by one or more natural antibody genes. For example, in one embodiment the parent antibody is an antibody that has been identified by affinity maturation.

The parent antibody can have affinity to any antigen known to those of skill in the art, or later discovered. Virtually any substance may be an antigen for a parent antibody, or an antibody of the present description. Examples of useful antigens include, but are not limited to, alpha-1 antitrypsin, angiostatin, antihemolytic factor, antibodies, apolipoprotein, apoprotein, atrial natriuretic factor, atrial natriuretic polypeptide, atrial peptides, C—X—C chemokines (e.g., T39765, NAP-2, ENA-78, Gro-a, Gro-b, Gro-c, IP-10, GCP-2, NAP-4, SDF-1, PF4, MIG), calcitonin, CC chemokines (e.g., monocyte chemoattractant protein-1, monocyte chemoattractant protein-2, monocyte chemoattractant protein-3, monocyte inflammatory protein-1 alpha, monocyte inflammatory protein-1 beta, RANTES, 1309, R83915, R91733, HCC1, T58847, D31065, T64262), CD40 ligand, C-kit ligand, collagen, colony stimulating factor (CSF), complement factor 5a, complement inhibitor, complement receptor 1, cytokines, (e.g., epithelial neutrophil activating peptide-78, GRO/MGSA, GRO, GRO, MIP-1, MIP-1, MCP-1), epidermal growth factor (EGF), erythropoietin (“EPO”), exfoliating toxins A and B, factor IX, factor VII, factor VIII, factor X, fibroblast growth factor (FGF), fibrinogen, fibronectin, G-CSF, GM-CSF, glucocerebrosidase, gonadotropin, growth factors, hedgehog proteins (e.g., Sonic, Indian, Desert), hemoglobin, hepatocyte growth factor (HGF), hirudin, human serum albumin, insulin, insulin-like growth factor (IGF), interferons (e.g., IFN-α, IFN-, IFN-γ), interleukins (e.g., IL-1, IL-2, IL-3, IL-4, IL-5, IL-6, IL-7, IL-8, IL-9, IL-10, IL-11, IL-12, etc.), keratinocyte growth factor (KGF), lactoferrin, leukemia inhibitory factor, luciferase, neurturin, neutrophil inhibitory factor (NIF), oncostatin M, osteogenic protein, parathyroid hormone, PD-ECSF, PDGF, peptide hormones (e.g., human growth hormone), pleiotropin, protein A, protein G, pyrogenic exotoxins A, B, and C, relaxin, renin, SCF, soluble complement receptor I, soluble I-CAM 1, soluble interleukin receptors (IL-1, 2, 3, 4, 5, 6, 7, 9, 10, 11, 12, 13, 14, 15), soluble TNF receptor, somatomedin, somatostatin, somatotropin, streptokinase, superantigens, i.e., staphylococcal enterotoxins (SEA, SEB, SEC1, SEC2, SEC3, SED, SEE), superoxide dismutase, toxic shock syndrome toxin (TSST-1), thymosin alpha 1, tissue plasminogen activator, tumor necrosis factor (TNFβ), tumor necrosis factor receptor (TNFR), tumor necrosis factor-alpha (TNFα), vascular endothelial growth factor (VEGF), urokinase and others. These antigens can be obtained by methods known to those of skill in the art, for example, from commercial sources or from published polypeptide or polynucleotide sequences (e.g. Genbank).

Additional antigens include, but are not limited to, transcriptional and expression activators. Exemplary transcriptional and expression activators include genes and proteins that modulate cell growth, differentiation, regulation, or the like. Expression and transcriptional activators are found in prokaryotes, viruses, and eukaryotes, including fungi, plants, and animals, including mammals, providing a wide range of therapeutic targets. It will be appreciated that expression and transcriptional activators regulate transcription by many mechanisms, e.g., by binding to receptors, stimulating a signal transduction cascade, regulating expression of transcription factors, binding to promoters and enhancers, binding to proteins that bind to promoters and enhancers, unwinding DNA, splicing pre-mRNA, polyadenylating RNA, and degrading RNA. Antigens include, but are not limited to, expression activators such as cytokines, inflammatory molecules, growth factors, their receptors, and oncogene products, e.g., interleukins (e.g., IL-1, IL-2, IL-8, etc.), interferons, FGF, IGF-I, IGF-II, FGF, PDGF, TNF, TGF-α, TGF-β, EGF, KGF, SCF/c-Kit, CD40L/CD40, VLA-4VCAM-1, ICAM-1/LFA-1, and hyalurin/CD44; signal transduction molecules and corresponding oncogene products, e.g., Mos, Ras, Raf, and Met; and transcriptional activators and suppressors, e.g., p53, Tat, Fos, Myc, Jun, Myb, Rel, and steroid hormone receptors such as those for estrogen, progesterone, testosterone, aldosterone, the LDL receptor ligand and corticosterone.

Vaccine proteins may be antigens including, but not limited to, proteins from infectious fungi, e.g., Aspergillus, Candida species; bacteria, particularly E. coli, which serves a model for pathogenic bacteria, as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B.

Antigens may be enzymes including, but not limited to, amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, and nucleases.

Agriculturally related proteins such as insect resistance proteins (e.g., the Cry proteins), starch and lipid production enzymes, plant and insect toxins, toxin-resistance proteins, Mycotoxin detoxification proteins, plant growth enzymes (e.g., Ribulose 1,5-Bisphosphate Carboxylase/Oxygenase, “RUBISCO”), lipoxygenase (LOX), and Phosphoenolpyruvate (PEP) carboxylase may also be antigens.

For example, the antigen may be a disease-associated molecule, such as tumor surface antigen such as B-cell idiotypes, CD20 on malignant B cells, CD33 on leukemic blasts, and HER2/neu on breast cancer. Alternatively, the antigen may be a growth factor receptor. Examples of the growth factors include, but are not limited to, epidermal growth factors (EGFs), transferrin, insulin-like growth factor, transforming growth factors (TGFs), interleukin-1, and interleukin-2. For example, a high expression of EGF receptors has been found in a wide variety of human epithelial primary tumors. TGF-α has been found to mediate an autocrine stimulation pathway in cancer cells. Several murine monoclonal antibodies have been demonstrated to be able to bind EGF receptors, block the binding of ligand to EGF receptors, and inhibit proliferation of a variety of human cancer cell lines in culture and in xenograft models. Mendelsohn and Baselga (1995) Antibodies to growth factors and receptors, in Biologic Therapy of Cancer, 2nd Ed., J B Lippincott, Philadelphia, pp 607-623. Thus, Antibodies of the invention may be used to treat a variety of cancers.

The antigen may also be cell surface protein or receptor associated with coronary artery disease such as platelet glycoprotein IIb/IIIa receptor, autoimmune diseases such as CD4, CAMPATH-1 and lipid A region of the gram-negative bacterial lipopolysaccharide. Humanized antibodies against CD4 have been tested in clinical trials in the treatment of patients with mycosis fungoides, generalized postular psoriasis, severe psoriasis, and rheumatoid arthritis. Antibodies against lipid A region of the gram-negative bacterial lipopolysaccharide have been tested clinically in the treatment of septic shock. Antibodies against CAMPATH-1 have also been tested clinically in the treatment of against refractory rheumatoid arthritis. Thus, antibodies provided herein may be used to treat a variety of autoimmune diseases.

Useful antigens also include proteins or peptides associated with human allergic diseases, such as inflammatory mediator proteins, e.g. interleukin-1 (IL-1), tumor necrosis factor (TNF), leukotriene receptor and 5-lipoxygenase, and adhesion molecules such as V-CAM/VLA-4. In addition, IgE may also serve as the antigen because IgE plays pivotal role in type I immediate hypersensitive allergic reactions such as asthma. Studies have shown that the level of total serum IgE tends to correlate with severity of diseases, especially in asthma. Burrows et al. (1989) “Association of asthma with serum IgE levels and skin-test reactivity to allergens” New Engl. L. Med. 320:271-277. Thus, Antibodies selected against IgE may be used to reduce the level of IgE or block the binding of IgE to mast cells and basophils in the treatment of allergic diseases without having substantial impact on normal immune functions.

The antigen may also be a viral surface or core protein which may serve as an antigen to trigger immune response of the infected host. Examples of these viral proteins include, but are not limited to, glycoproteins (or surface antigens, e.g., GP120 and GP41) and capsid proteins (or structural proteins, e.g., P24 protein); surface antigens or core proteins of hepatitis A, B, C, D or E virus (e.g. small hepatitis B surface antigen (SHBsAg) of hepatitis B virus and the core proteins of hepatitis C virus, NS3, NS4 and NS5 antigens); glycoprotein (G-protein) or the fusion protein (F-protein) of respiratory syncytial virus (RSV); surface and core proteins of herpes simplex virus HSV-1 and HSV-2 (e.g., glycoprotein D from HSV-2).

The antigen may also be a mutated tumor suppressor gene product that has lost its tumor-suppressing function and may render the cells more susceptible to cancer. Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, DPC-4, NF-1, NF-2, RB, p53, WT1, BRCA1 and BRCA2. DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. p53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. Thus, Antibodies may be used to block the interactions of the gene product with other proteins or biochemicals in the pathways of tumor onset and development.

The antigen may be a CD molecule including but not limited to, CD1a, CD1b, CD1c, CD1d, CD2, CD3γ, CD3δ, CD3ε, CD4, CD5, CD6, CD7, CD8α, CD8β, CD9, CD10, CD11a, CD11b, CD11c, CDw12, CD13, CD14, CD15, CD15s, CD16a, CD16b, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45R, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD67, CD68, CD69, CDw70, CD71, CD72, CD73, CD74, CDw75, CDw76, CD77, CD79α, CD79β, CD80, CD81, CD82, CD83, CD84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CDw109, CD110-113, CD114, CD115, CD116, CD117, CD118, CD119, CD120a, CD120b, CD121a, CD121b, CD122, CD123, CDw124, CD125, CD126, CDw127, CDw128a, CDw128b, CD129, CDw130, CD131, CD132, CD133, CD134, CD135, CD136, CDw137, CD138, CD139, CD140a, CD140b, CD141, CD142, CD143, CD144, CDw145, CD146, CD147, CD148, CDw149, CD150, CD151, CD152, CD153, CD154, CD155, CD156, CD157, CD158a, CD158b, CD161, CD162, CD163, CD164, CD165, CD166, and TCRζ. The antigen may be VEGF, VEGF receptor, EGFR, Her2, TNFa, TNFRI receptor, GPIIb/IIIa, IL-2Rα chain, IL-2Rβ chain, RSV F protein, alpha-4 integrin, IgE, IgE receptor, digoxin, carpet viper venom, complement C5, OPGL, CA-125 tumor antigen, Staphylococci proteins, Staphylococcus epidermidis proteins, Staphylococcus aureus proteins, proteins involved Staphylococcal infection (including but not limited to, Staphylococcus aureus and Staphylococcus epidermidis), IL-6 receptor, CTLA-4, RSV, Tac subunit of IL-2 receptor, IL-5, and EpCam. The antigen may be a fragment of a molecule.

Parent antibodies can be any antibody known in the art or any antibody discovered or developed by those of skill in the art without limitation. Examples include, but are not limited to anti-TNF antibody (U.S. Pat. No. 6,258,562), anti-IL-12 and/or anti-IL-12p40 antibody (U.S. Pat. No. 6,914,128); anti-IL-18 antibody (U.S. Patent Publication No. 2005/0147610), anti-05, anti-CBL, anti-CD147, anti-gp120, anti-VLA-4, anti-CD11a, anti-CD18, anti-VEGF, anti-CD40L, anti CD-40 (e.g., see PCT Publication No. WO 2007/124299) anti-Id, anti-ICAM-1, anti-CXCL13, anti-CD2, anti-EGFR, anti-TGF-β 2, anti-HGF, anti-cMet, anti DLL-4, anti-NPRI, anti-PLGF, anti-ErbB3, anti-E-selectin, anti-Fact VII, anti-Her2/neu, anti-F gp, anti-CD11/18, anti-CD14, anti-ICAM-3, anti-RON, anti-SOST, anti CD-19, anti-CD80 (e.g., see PCT Publication No. WO 2003/039486, anti-CD4, anti-CD3, anti-CD23, anti-P2-integrin, anti-α4β7, anti-CD52, anti-HLA DR, anti-CD22 (e.g., see U.S. Pat. No. 5,789,554), anti-CD20, anti-MIF, anti-CD64 (FcR), anti-TCR α and/or β, anti-CD2, anti-Hep B, anti-CA 125, anti-EpCAM, anti-gp120, anti-CMV, anti-gpIIbIIIa, anti-IgE, anti-CD25, anti-CD33, anti-HLA, anti-IGF1,2, anti IGFR, anti-VNRintegrin, anti-IL-1α, anti-IL-1β, anti-IL-1 receptor, anti-IL-2 receptor, anti-IL-4, anti-IL-4 receptor, anti-ILS, anti-IL-5 receptor, anti-IL-6, anti-IL-8, anti-IL-9, anti-IL-13, anti-IL-13 receptor, anti-IL-17, anti-IL-6R, anti-RANKL, anti-NGF, anti-DKK, anti-αVβ3, anti-IL-17A, anti-IL23p19 and anti-IL-23 (see Presta, L. G. (2005) J. Allergy Clin. Immunol. 116: 731-6 and www<dot>path<dot>cam<dot>ac<dot>uk</><dot>about<dot>mrc<7></>humanisation</>antibodies<dot>html).

Parent antibodies may also be selected from various therapeutic antibodies approved for use, in clinical trials, or in development for clinical use. Such therapeutic antibodies include, but are not limited to, rituximab (Rituxan®, IDEC/Genentech/Roche) (see, for example, U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PRO70769 (PCT Application No. PCT/US2003/040426), trastuzumab (Herceptin®, Genentech) (see, for example, U.S. Pat. No. 5,677,171), a humanized anti-Her2/neu antibody approved to treat breast cancer; pertuzumab (rhuMab-2C4, Omnitarg®), currently being developed by Genentech; an anti-Her2 antibody (U.S. Pat. No. 4,753,894; cetuximab (Erbitux®, Imclone) (U.S. Pat. No. 4,943,533; PCT Publication No. WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Pat. No. 7,247,301), currently being developed by Genmab; 425, EMD55900, EMD62000, and EMD72000 (Merck KGaA) (U.S. Pat. No. 5,558,864; Murthy, et al. (1987) Arch. Biochem. Biophys. 252(2): 549-60; Rodeck, et al. (1987) J. Cell. Biochem. 35(4): 315-20; Kettleborough, et al. (1991) Protein Eng. 4(7): 773-83); ICR62 (Institute of Cancer Research) (PCT Publication No. WO 95/20045; Modjtahedi, et al. (1993) J. Cell. Biophys. 22(I-3): 129-46; Modjtahedi, et al. (1993) Br. J. Cancer 67(2): 247-53; Modjtahedi, et al. (1996) Br. J. Cancer 73(2): 228-35; Modjtahedi, et al. (2003) Int. J. Cancer 105(2): 273-80); TheraCIM hR3 (YM Biosciences, Canada and Centro de Immunologia Molecular, Cuba (U.S. Pat. Nos. 5,891,996; 6,506,883; Mateo, et al. (1997) Immunotechnol. 3(1): 71-81); mAb-806 (Ludwig Institute for Cancer Research, Memorial Sloan-Kettering) (Jungbluth, et al. (2003) Proc. Natl. Acad. Sci. USA. 100(2): 639-44); KSB-102 (KS Biomedix); MR1-1 (IVAX, National Cancer Institute) (PCT Publication No. WO 01/62931A2); and SC100 (Scancell) (PCT Publication No. WO 01/88138); alemtuzumab (Campath®, Millenium), a humanized mAb currently approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amevive®), an anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFa antibody developed by Centocor, adalimumab (Humira®), an anti-TNFa antibody developed by Abbott, Humicade®, an anti-TNFa antibody developed by Celltech, golimumab (CNTO-148), a fully human TNF antibody developed by Centocor, etanercept (Enbrel®), an p75 TNF receptor Fc fusion developed by Immunex/Amgen, Ienercept, an p55TNF receptor Fc fusion previously developed by Roche, ABX-CBL, an anti-CD147 antibody being developed by Abgenix, ABX-IL8, an anti-IL8 antibody being developed by Abgenix, ABX-MA1, an anti-MUC18 antibody being developed by Abgenix, Pemtumomab (R1549, 90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegren® (natalizumab), an anti-α-4-β-1 (VLA-4) and α-4-β-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-0 antibody being developed by Cambridge Antibody Technology, ABT 874 (J695), an anti-IL-12 p40 antibody being developed by Abbott, CAT-192, an anti-TGFβ1 antibody being developed by Cambridge Antibody Technology and Genzyme, CAT-213, an anti-Eotaxin1 antibody being developed by Cambridge Antibody Technology, LymphoStat-B® an anti-Blys antibody being developed by Cambridge Antibody Technology and Human Genome Sciences Inc., TRAIL-R1 mAb, an anti-TRAIL-R1 antibody being developed by Cambridge Antibody Technology and Human Genome Sciences, Inc., Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech, Xolair® (Omalizumab), an anti-IgE antibody being developed by Genentech, Raptiva® (Efalizumab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDEC Pharmaceuticals, IDEC-152, an anti-CD 23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (Iabetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-1), and anti-Her2 antibody being developed by Medarex and Immuno-Designed Molecules, HuMax®-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J, CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAF®, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLN01, an anti-β2 integrin antibody being developed by Xoma. In another embodiment, the therapeutics include KRN330 (Kirin); huA33 antibody (A33, Ludwig Institute for Cancer Research); CNTO 95 (alpha V integrins, Centocor); MEDI-522 (αVβ3 integrin, Medimmune); volociximab (αVβ1 integrin, Biogen/PDL); Human mAb 216 (B cell glycosolated epitope, NCl); BiTE MT103 (bispecific CD19×CD3, Medimmune); 4G7×H22 (Bispecific Bcell×FcγR1, Medarex/Merck KGa); rM28 (Bispecific CD28×MAPG, EP Patent No. EP1444268); MDX447 (EMD 82633) (Bispecific CD64×EGFR, Medarex); Catumaxomab (removab) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCl); MDX-060 (CD30, Medarex); MDX-1401 (CD30, Medarex); SGN-30 (CD30, Seattle Genentics); SGN-33 (Lintuzumab) (CD33, Seattle Genentics); Zanolimumab (HuMax-CD4) (CD4, Genmab); HCD122 (CD40, Novartis); SGN-40 (CD40, Seattle Genentics); Campath1h (Alemtuzumab) (CD52, Genzyme); MDX-1411 (CD70, Medarex); hLL1 (EPB-1) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb); Tremelimumab (Ticilimumab, CP-675,2) (CTLA4, Pfizer); HGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam, Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DRP chain, PDL Pharma); AMG-479 (IGF-1R, Amgen); anti-IGF-1R R1507 (IGF1-R, Roche); CP 751871 (IGF1-R, Pfizer); IMC-A12 (IGF1-R, Imclone); BIIB022 (IGF-1R, Biogen); Mik-P-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTOR, Biogen); HuHMFG1 (MUC1, Antisoma/NCl); RAV12 (N-linked carbohydrate epitope, Raven); CAL (parathyroid hormone-related protein (PTH-rP), University of California); CT-011 (PD1, CureTech); MDX-1106 (ono-4538) (PD1, Medarex/Ono); MAb CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFa, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab, PCT Publication No. WO/2000/034337, University of Texas); IMC-18F1 (VEGFR1, Imclone); IMC-1121 (VEGFR2, Imclone).

Examples of useful bispecific parent antibodies include, but are not limited to, those with one antibody directed against a tumor cell antigen and the other antibody directed against a cytotoxic trigger molecule such as anti-FcγRI/anti-CD 15, anti-p185^{HER2}/FcγRIII (CD16), anti-CD3/anti-malignant B-cell (1D10), anti-CD3/anti-p185^{HER2}, anti-CD3/anti-p97, anti-CD3/anti-renal cell carcinoma, anti-CD3/anti-OVCAR-3, anti-CD3/L-D1 (anti-colon carcinoma), anti-CD3/anti-melanocyte stimulating hormone analog, anti-EGF receptor/anti-CD3, anti-CD3/anti-CAMA1, anti-CD3/anti-CD19, anti-CD3/MoV18, anti-neural cell adhesion molecule (NCAM)/anti-CD3, anti-folate binding protein (FBP)/anti-CD3, anti-pan carcinoma associated antigen (AMOC-31)/anti-CD3; bispecific antibodies with one antibody which binds specifically to a tumor antigen and another antibody which binds to a toxin such as anti-saporin/anti-Id-1, anti-CD22/anti-saporin, anti-CD7/anti-saporin, anti-CD38/anti-saporin, anti-CEA/anti-ricin A chain, anti-interferon-α (IFN-α)/anti-hybridoma idiotype, anti-CEA/anti-vinca alkaloid; bispecific antibodies for converting enzyme activated prodrugs such as anti-CD30/anti-alkaline phosphatase (which catalyzes conversion of mitomycin phosphate prodrug to mitomycin alcohol); bispecific antibodies which can be used as fibrinolytic agents such as anti-fibrin/anti-tissue plasminogen activator (tPA), anti-fibrin/anti-urokinase-type plasminogen activator (uPA); bispecific antibodies for targeting immune complexes to cell surface receptors such as anti-low density lipoprotein (LDL)/anti-Fc receptor (e.g. FcγRI, FcγRII or FcγRIII); bispecific antibodies for use in therapy of infectious diseases such as anti-CD3/anti-herpes simplex virus (HSV), anti-T-cell receptor:CD3 complex/anti-influenza, anti-FcγR/anti-HIV; bispecific antibodies for tumor detection in vitro or in vivo such as anti-CEA/anti-EOTUBE, anti-CEA/anti-DPTA, anti-anti-p185^{HER2}/anti-hapten; bispecific antibodies as vaccine adjuvants (see Fanger, M W et al., Crit Rev Immunol. 1992; 12(34):101-24, which is incorporated by reference herein); and bispecific antibodies as diagnostic tools such as anti-rabbit IgG/anti-ferritin, anti-horse radish peroxidase (HRP)/anti-hormone, anti-somatostatin/anti-substance P, anti-HRP/anti-FITC, anti-CEA/anti-p-galactosidase (see Nolan, O et R. O'Kennedy, Biochim Biophys Acta. 1990 Aug. 1; 1040(1):1-11, which is incorporated by reference herein). Examples of trispecific antibodies include anti-CD3/anti-CD4/anti-CD37, anti-CD3/anti-CD5/anti-CD37 and anti-CD3/anti-CD8/anti-CD37.

Antibody and Conjugate Compositions

Antibodies and conjugates described herein can be formulated into compositions using methods available in the art and those disclosed herein. Any of the compounds disclosed herein can be provided in the appropriate pharmaceutical composition and be administered by a suitable route of administration.

In certain embodiments, the antibody and conjugate compositions provided herein further comprise a pharmaceutically acceptable carrier. The carrier can be a diluent, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in E. W. Martin, 1990, Remington's Pharmaceutical Sciences, Mack Publishing Co.

In some embodiments, the pharmaceutical composition is provided in a form suitable for administration to a human subject. In some embodiments, the pharmaceutical composition will contain a prophylactically or therapeutically effective amount of the antibody together with a suitable amount of carrier so as to provide the form for proper administration to the patient. The formulation should suit the mode of administration.

In some embodiments, the pharmaceutical composition is provided in a form suitable for intravenous administration. Typically, compositions suitable for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous administration.

In particular embodiments, the pharmaceutical composition is suitable for subcutaneous administration. In particular embodiments, the pharmaceutical composition is suitable for intramuscular administration.

Components of the pharmaceutical composition can be supplied either separately or mixed together in unit dosage form, for example, as a dry lyophilized powder or water free concentrate. Where the composition is to be administered by infusion, it can be dispensed with an infusion bottle containing sterile pharmaceutical grade water or saline. Where the composition is administered by injection, an ample of sterile water for injection or saline can be provided so that the ingredients may be mixed prior to administration.

In some embodiments, the pharmaceutical composition is supplied as a dry sterilized lyophilized powder that is capable of being reconstituted to the appropriate concentration for administration to a subject. In some embodiments, antibodies are supplied as a water free concentrate. In some embodiments, the antibody is supplied as a dry sterile lyophilized powder at a unit dosage of at least 0.5 mg, at least 1 mg, at least 2 mg, at least 3 mg, at least 5 mg, at least 10 mg, at least 15 mg, at least 25 mg, at least 30 mg, at least 35 mg, at least 45 mg, at least 50 mg, at least 60 mg, or at least 75 mg.

In another embodiment, the pharmaceutical composition is supplied in liquid form. In some embodiments, the pharmaceutical composition is provided in liquid form and is substantially free of surfactants and/or inorganic salts. In some embodiments, the antibody is supplied as in liquid form at a unit dosage of at least 0.1 mg/ml, at least 0.5 mg/ml, at least 1 mg/ml, at least 2.5 mg/ml, at least 3 mg/ml, at least 5 mg/ml, at least 8 mg/ml, at least 10 mg/ml, at least 15 mg/ml, at least 25 mg/ml, at least 30 mg/ml, or at least 60 mg/ml.

In some embodiments, the pharmaceutical composition is formulated as a salt form. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

In therapeutic use, the practitioner will determine the posology most appropriate according to a preventive or curative treatment and according to the age, weight, stage of the infection and other factors specific to the subject to be treated. In certain embodiments, doses are from about 1 to about 1000 mg per day for an adult, or from about 5 to about 250 mg per day or from about 10 to 50 mg per day for an adult. In certain embodiments, doses are from about 5 to about 400 mg per day or 25 to 200 mg per day per adult. In certain embodiments, dose rates of from about 50 to about 500 mg per day are also contemplated.

Methods of Use for Therapy or Prophylaxis

Certain antibodies and conjugates provided herein can be used for the treatment or prevention of any disease or condition deemed suitable to the practitioner of skill in the art. Generally, a method of treatment or prevention encompasses the administration of a therapeutically or prophylactically effective amount of the antibody, conjugate, or composition to a subject in need thereof to treat or prevent the disease or condition.

A therapeutically effective amount of the antibody, conjugate, or composition is an amount that is effective to reduce the severity, the duration and/or the symptoms of a particular disease or condition. The amount of the antibody, conjugate, or composition that will be therapeutically effective in the prevention, management, treatment and/or amelioration of a particular disease can be determined by standard clinical techniques. The precise amount of the antibody or composition to be administered with depend, in part, on the route of administration, the seriousness of the particular disease or condition, and should be decided according to the judgment of the practitioner and each subject's circumstances.

In some embodiments, the effective amount of the antibody or conjugate provided herein is between about 0.025 mg/kg and about 1000 mg/kg body weight of a human subject. In certain embodiments, the antibody is administered to a human subject at an amount of about 1000 mg/kg body weight or less, about 950 mg/kg body weight or less, about 900 mg/kg body weight or less, about 850 mg/kg body weight or less, about 800 mg/kg body weight or less, about 750 mg/kg body weight or less, about 700 mg/kg body weight or less, about 650 mg/kg body weight or less, about 600 mg/kg body weight or less, about 550 mg/kg body weight or less, about 500 mg/kg body weight or less, about 450 mg/kg body weight or less, about 400 mg/kg body weight or less, about 350 mg/kg body weight or less, about 300 mg/kg body weight or less, about 250 mg/kg body weight or less, about 200 mg/kg body weight or less, about 150 mg/kg body weight or less, about 100 mg/kg body weight or less, about 95 mg/kg body weight or less, about 90 mg/kg body weight or less, about 85 mg/kg body weight or less, about 80 mg/kg body weight or less, about 75 mg/kg body weight or less, about 70 mg/kg body weight or less, or about 65 mg/kg body weight or less.

In some embodiments, the effective amount of antibody or conjugate provided herein is between about 0.025 mg/kg and about 60 mg/kg body weight of a human subject. In some embodiments, the effective amount of an antibody of the pharmaceutical composition provided herein is about 0.025 mg/kg or less, about 0.05 mg/kg or less, about 0.10 mg/kg or less, about 0.20 mg/kg or less, about 0.40 mg/kg or less, about 0.80 mg/kg or less, about 1.0 mg/kg or less, about 1.5 mg/kg or less, about 3 mg/kg or less, about 5 mg/kg or less, about 10 mg/kg or less, about 15 mg/kg or less, about 20 mg/kg or less, about 25 mg/kg or less, about 30 mg/kg or less, about 35 mg/kg or less, about 40 mg/kg or less, about 45 mg/kg or less, about 50 mg/kg or about 60 mg/kg or less.

The pharmaceutical composition of the method can be administered using any method known to those skilled in the art. For example, the pharmaceutical composition can be administered intramuscularly, intradermally, intraperitoneally, intravenously, subcutaneously administration, or any combination thereof. In some embodiments, the pharmaceutical composition is administered subcutaneously. In some embodiments, the composition is administered intravenously. In some embodiments, the composition is administered intramuscularly.

Methods of Use for Detection or Diagnosis

The antibodies and conjugates provided herein can be used for the detection of any target or for the diagnosis of any disease or condition deemed suitable to the practitioner of skill in the art. The methods encompass detecting the binding of an antibody or conjugate to a target antigen in the appropriate location, e.g., the appropriate body, tissue, or cell. In the methods, the formation of a complex between the antibody and antigen can be detected by any method known to those of skill in the art. Examples include assays that use secondary reagents for detection, ELISA's and immunoprecipitation and agglutination assays. A detailed description of these assays is, for example, given in Harlow and Lane, Antibodies: A Laboratory Manual (Cold Spring Harbor Laboratory, New York 1988 555-612, WO96/13590 to Maertens and Stuyver, Zrein et al. (1998) and WO96/29605.

For in situ diagnosis, the antibody or conjugate may be administered to a subject by methods known in the art such as, for example, intravenous, intranasal, intraperitoneal, intracerebral, intraarterial injection such that a specific binding between an antibody according to the invention with an eptitopic region on the amyloid protein may occur. The antibody/antigen complex may conveniently be detected through a label attached to the antibody or any other art-known method of detection.

Further provided herein are kits for detection or diagnosis. Exemplary kits comprise one or more antibodies or conjugates provided herein along with one or more reagents useful for detecting a complex between the one or more antibodies or conjugates and their target antigens.

Preparation of Antibodies

The antibodies described herein can be prepared by any technique apparent to those of skill in the art without limitation. Useful techniques for preparation include in vivo synthesis, for example with modified tRNA and tRNA synthetase, cell-free synthesis, for example with modified tRNA and tRNA synthetase, solid phase polypeptide synthesis and liquid phase polypeptide synthesis. Exemplary techniques are described in this section and in the examples below.

In certain methods, the antibody is translated and/or transcribed from one or more polynucleotides encoding the polypeptide chains of the antibody. Accordingly, provided herein are polynucleotides capable of encoding the antibodies having one or more non-natural amino acids at site-specific positions in one or more polypeptide chains. In certain embodiments, the polynucleotides comprise a codon not normally associated with an amino acid at the polynucleotide position corresponding to the site-specific polypeptide position for the non-natural amino acid. Examples of such codons include stop codons, 4 bp codons, 5 bp codons, and the like. The reaction mixture typically comprises a tRNA synthetase capable of making tRNAs that complement (suppress) said codon. These suppressor tRNAs are linked to the non-natural amino acids to facilitate their incorporation into the polypeptide at the site of the suppressor codon.

The antibodies can be prepared by techniques known to those of skill in the art for expressing such polynucleotides to incorporate non-natural amino acids into site specific positions of a polypeptide chain. Such techniques are described, for example, in U.S. Pat. Nos. 7,045,337 and 7,083,970, in U.S. Published Patent Application Nos. US 2008/0317670, US 2009/0093405, US 2010/0093082, US 2010/0098630, US 2008/0085277 and in international patent publication nos. WO 2004/016778 A1 and WO 2008/066583 A2, the contents of which are hereby incorporated by reference in their entireties.

In certain embodiments, an antibody can be prepared in a cell-free reaction mixture comprising at least one orthogonal tRNA aminoacylated with an unnatural amino acid, where the orthogonal tRNA base pairs with a codon that is not normally associated with an amino acid, e.g. a stop codon; a 4 bp codon, etc. The reaction mixture also comprises a tRNA synthetase capable of aminoacylating the orthogonal tRNA with an unnatural amino acid. Usually the orthogonal tRNA synthetase, which is susceptible to degradation by proteases present in bacterial cell extracts, is exogenously synthesized and added to the reaction mix prior to initiation of polypeptide synthesis. The orthogonal tRNA may be synthesized in the bacterial cells from which the cell extract is obtained, may be synthesized de novo during the polypeptide synthesis reaction, or may be exogenously added to the reaction mix.

In certain embodiments, a variant of the aminoacyl tRNA synthetase provided in SEQ ID NO: 5 is used to catalyze the attachment of a non-natural amino acid to a compatible tRNA. Variants of the aminoacyl tRNA synthetase of SEQ ID NO: 5 are particularly advantageous when utilizing amino acids comprising tetrazine functional groups, such as those provided in any of formulas AI, AIa, AII, AIII, AIV, AV, AVI, AVII, AVIII, AIX, and (A1)-(A10). In certain embodiments, a variant of SEQ ID NO: 3 with the following mutations, designated “2A2”, may be particularly advantageous for use with a non-natural amino acid of formula A9: Y32L, L65V, H70A, F108W, Q109S, D158V, I159A, and L162V (SEQ ID NO: 6). In some embodiments, a variant of SEQ ID NO: 3 with the following mutations, designated “2A9”, may be particularly advantageous for use with a non-natural amino acid of formula A6: Y32G, L65V, H70A, Q109S, D158G, and L162S (SEQ ID NO: 7). Other aminoacyl tRNA synthetases that may be useful with the compounds of the invention include the mtaF synthetase disclosed in Seitchik et al., J. Am. Chem. Soc., 2012, 134:2898-2901 (incorporated by reference in its entirety) and other variants of SEQ ID NO: 5. Variants of SEQ ID NO: 3 may be made by mutagenesis and screened to identify mutant synthetases that act on any non-natural amino acid of interest. Such mutagenesis may be completely random, or may be deterministic with respect to the location of the mutation(s) and/or the residue(s) allowed to occur at a particular portion of the synthetase polypeptide sequence. Examples of methods for random mutagenesis of synthetases may be found in Seitchik et al., cited above and incorporated by reference in its entirety.

In certain embodiments, components that affect unnatural amino acid insertion and protein insertion or folding are optionally added to the reaction mixture. Such components include elevated concentrations of translation factors to minimize the effect of release factor 1 and 2 and to further optimize orthogonal component concentrations. Protein chaperones (Dsb System of oxidoreductases and isomerases, GroES, GroEL, DNAJ, DNAK, Skp, etc.) may be exogenously added to the reaction mixture or may be overexpressed in the source cells used to prepare the cell extract The reactions may utilize a large scale reactor, small scale, or may be multiplexed to perform a plurality of simultaneous syntheses. Continuous reactions will use a feed mechanism to introduce a flow of reagents, and may isolate the end-product as part of the process. Batch systems are also of interest, where additional reagents may be introduced to prolong the period of time for active synthesis. A reactor may be run in any mode such as batch, extended batch, semi-batch, semi-continuous, fed-batch and continuous, and which will be selected in accordance with the application purpose. The reactions may be of any volume, either in a small scale, usually at least about 1 μl and not more than about 15 μl, or in a scaled up reaction, where the reaction volume is at least about 15 μl, usually at least about 50 μl, more usually at least about 100 μl, and may be 500 μl, 1000 μl, or greater. In principle, reactions may be conducted at any scale as long as sufficient oxygen (or other electron acceptor) is supplied when needed.

Useful methods for synthesis where at least one unnatural amino acid is introduced into the polypeptide strand during elongation include but are not limited to: (I) addition of exogenous purified orthogonal synthetase, unnatural amino acid, and orthogonal tRNA to the cell-free reaction, (II) addition of exogenous purified orthogonal synthetase and unnatural amino acid to the reaction mixture, but with orthogonal tRNA transcribed during the cell-free reaction, (III) addition of exogenous purified orthogonal synthetase and unnatural amino acid to the reaction mixture, but with orthogonal tRNA synthesized by the cell extract source organism. In certain embodiments, the orthogonal components are driven by regulatable promoters, so that synthesis levels can be controlled although other measures may be used such as controlling the level of the relevant DNA templates by addition or specific digestion.

In some embodiments, a bacterial cell-free expression system is used to produce protein or peptide variants with non-native amino acids (nnAA). The use of bacterial cell-free extracts for in vitro protein synthesis offers several advantages over conventional in vivo protein expression methods. Cell-free systems can direct most, if not all, of the metabolic resources of the cell towards the exclusive production of one protein. Moreover, the lack of a cell wall and membrane components in vitro is advantageous since it allows for control of the synthesis environment. However, the efficiency of cell-free extracts can be decreased by bacterial proteins that inhibit protein synthesis, either directly or indirectly. Thus, inactivation of undesirable proteins that decrease the efficiency of protein synthesis should increase the yield of desirable proteins in cell-free extracts. For example, the inactivation of proteins that decrease the efficiency of protein synthesis should increase the yield of polypeptides having non-native amino acids incorporated at a defined amino acid residue. The introduction of nnAA into polypeptides is useful for increasing the biological diversity and function of proteins. One approach for producing polypeptides having a nnAA incorporated at a defined amino acid residue is to use an nnAA, aminoacylated orthogonal CUA containing tRNA for introduction of the nnAA into the nascent polypeptide at an amber (stop) codon during protein translation. However, the incorporation of nnAA at an amber codon can be inhibited by the native bacterial termination complex, which normally recognizes the stop codon and terminates translation. Release Factor 1 (RF1) is a termination complex protein that facilitates the termination of translation by recognizing the amber codon in an mRNA sequence. RF1 recognition of the amber stop codon can promote pre-mature truncation products at the site of non-native amino acid incorporation, and thus decreased protein yield. Therefore, attenuating the activity of RF 1 may increase nnAA incorporation into recombinant proteins.

It has previously been shown that nnAA incorporation can be increased by attenuating RF1 activity in 3 ways: 1) neutralizing antibody inactivation of RF1, 2) genomic knockout of RF1 (in an RF2 bolstered strain), and 3) site specific removal of RF1 using a strain engineered to express RF1 containing a protein tag for removal by affinity chromatography (Chitin Binding Domain and His Tag). Another method for inactivating RF1 comprises introducing proteolytic cleavage sites into the RF1 amino acid sequence. The cleavage sites are not accessible to the protease during bacterial cell growth, but are cleaved by the protease when the bacterial cells are lysed to produce cell-free extract. Thus, the yield of full length polypeptides having a nnAA incorporated at an amber codon is increased in bacterial cell extracts expressing such modified RF1 variants.

In some embodiments, in order to produce antibodies comprising a non-natural amino acid, one needs a nucleic acid template. The templates for cell-free protein synthesis can be either mRNA or DNA. The template can comprise sequences for any particular antibody of interest, and may encode a full-length antibody or a fragment of any length thereof. Nucleic acids that serve as protein synthesis templates are optionally derived from a natural source or they can be synthetic or recombinant. For example, DNAs can be recombinant DNAs, e.g., plasmids, viruses or the like.

In some embodiments, once a nucleic acid template of an antibody is produced, the template is used to synthesize the antibody in a cell-free translation system. For example, the template can be added to a cell lysate under conditions sufficient to translate the template into protein. The cell lysate can be from bacterial cells or eukaryotic cells. The expressed protein can then be purified using methods known in the art, as described below.

In some embodiments, a translation system (e.g., an in vitro protein synthesis system) is used to produce the antibody with one or more nnAAs incorporated therein. An exemplary translation system comprises a cell free extract, cell lysate, or reconstituted translation system, along with the nucleic acid template for synthesis of the desired polypeptide or protein having non-native amino acids at preselected (defined) positions. The reaction mixture will further comprise monomers for the macromolecule to be synthesized, e.g. amino acids, nucleotides, etc., and such co-factors, enzymes and other reagents that are necessary for the synthesis, e.g. ribosomes, tRNA, polymerases, transcriptional factors, etc. In addition to the above components such as a cell-free extract, nucleic acid template, and amino acids, materials specifically required for protein synthesis may be added to the reaction. The materials include salts, folinic acid, cyclic AMP, inhibitors for protein or nucleic acid degrading enzymes, inhibitors or regulators of protein synthesis, adjusters of oxidation/reduction potentials, non-denaturing surfactants, buffer components, spermine, spermidine, putrescine, etc. Various cell-free synthesis reaction systems are well known in the art. See, e.g., Kim, D. M. and Swartz, J. R. Biotechnol. Bioeng. 66:180-8 (1999); Kim, D. M. and Swartz, J. R. Biotechnol. Prog. 16:385-90 (2000); Kim, D. M. and Swartz, J. R. Biotechnol. Bioeng. 74:309-16 (2001); Swartz et al, Methods Mol Biol. 267:169-82 (2004); Kim, D. M. and Swartz, J. R. Biotechnol. Bioeng. 85:122-29 (2004); Jewett, M. C. and Swartz, J. R., Biotechnol. Bioeng. 86:19-26 (2004); Yin, G. and Swartz, J. R., Biotechnol. Bioeng. 86:188-95 (2004); Jewett, M. C. and Swartz, J. R., Biotechnol. Bioeng. 87:465-72 (2004); Voloshin, A. M. and Swartz, J. R., Biotechnol. Bioeng. 91:516-21 (2005). Additional conditions for the cell-free synthesis of desired polypeptides are described in WO2010/081110, the contents of which are incorporated by reference herein in its entirety.

In some embodiments, a DNA template is used to drive in vitro protein synthesis, and RNA polymerase is added to the reaction mixture to provide enhanced transcription of the DNA template. RNA polymerases suitable for use herein include any RNA polymerase that functions in the bacteria from which the bacterial extract is derived. In other embodiments, an RNA template is used to drive in vitro protein synthesis, and the components of the reaction mixture can be admixed together in any convenient order, but are preferably admixed in an order wherein the RNA template is added last, thereby minimizing potential degradation of the RNA template by nucleases.

In some embodiments, a cell-free translation system is used to produce the antibody with one or more nnAAs incorporated therein. Cell-free protein synthesis exploits the catalytic power of the cellular machinery. Obtaining maximum protein yields in vitro requires adequate substrate supply, e.g. nucleoside triphosphates and amino acids, a homeostatic environment, catalyst stability, and the removal or avoidance of inhibitory byproducts. The optimization of in vitro synthetic reactions benefits from recreating the in vivo state of a rapidly growing organism. In some embodiments of the invention, cell-free synthesis is therefore performed in a reaction where oxidative phosphorylation is activated. Additional details are described in U.S. Pat. No. 7,338,789, the contents of which are incorporated by reference herein in its entirety.

In vitro, or cell-free, protein synthesis offers several advantages over conventional in vivo protein expression methods. Cell-free systems can direct most, if not all, of the metabolic resources of the cell towards the exclusive production of one protein. Moreover, the lack of a cell wall and membrane components in vitro is advantageous since it allows for control of the synthesis environment. For example, tRNA levels can be changed to reflect the codon usage of genes being expressed. The redox potential, pH, or ionic strength can also be altered with greater flexibility than with in vivo protein synthesis because concerns of cell growth or viability do not exist. Furthermore, direct recovery of purified, properly folded protein products can be easily achieved. In some embodiments, the productivity of cell-free systems has improved over 2-orders of magnitude in recent years, from about 5 pg/ml-hr to about 500 pg/ml-hr.

In certain embodiments, tRNA synthetase is exogenously synthesized and added to the cell-free reaction mix. In certain embodiments, the reaction mix is prepared from bacterial cells in which ompT has been inactivated or is naturally inactive. OmpT is believed to degrade components of the reaction mixture including tRNA synthetase.

In addition to the above components such as cell-free extract, genetic template, and amino acids, materials specifically required for protein synthesis may be added to the reaction. These materials include salts, folinic acid, cyclic AMP, inhibitors for protein or nucleic acid degrading enzymes, inhibitors or regulators of protein synthesis, adjusters of oxidation/reduction potential(s), non-denaturing surfactants, buffer components, spermine, spermidine, putrescine, etc.

The salts preferably include potassium, magnesium, and ammonium salts (e.g. of acetic acid or glutamic acid). One or more of such salts may have an alternative amino acid as a counter anion. There is an interdependence among ionic species for optimal concentration. These ionic species are typically optimized with regard to protein production. When changing the concentration of a particular component of the reaction medium, that of another component may be changed accordingly. For example, the concentrations of several components such as nucleotides and energy source compounds may be simultaneously adjusted in accordance with the change in those of other components. Also, the concentration levels of components in the reactor may be varied over time. The adjuster of oxidation/reduction potential may be dithiothreitol, ascorbic acid, glutathione and/or their oxidized forms.

In certain embodiments, the reaction can proceed in a dialysis mode, in a diafiltration batch mode, in a fed-batch mode of in a semi-continuous operation mode. In certain embodiments, a feed solution can be supplied to the reactor through a membrane or through an injection unit. Synthesized antibody can accumulate in the reactor followed by isolation or purification after completion of the system operation. Vesicles containing the antibody may also be continuously isolated, for example by affinity adsorption from the reaction mixture either in situ or in a circulation loop as the reaction fluid is pumped past the adsorption matrix.

During protein synthesis in the reactor, the protein isolating means for selectively isolating the desired protein may include a unit packed with particles coated with antibody molecules or other molecules for adsorbing the synthesized, desired protein. Preferably, the protein isolating means comprises two columns for alternating use.

The resulting antibody can be purified or isolated by standard techniques. Exemplary techniques are provided in the examples below.

Assay Methods

Antibodies can be assayed for their expected activity, or for a new activity, according to any assay apparent to those of skill in the art. The resulting antibody can be assayed activity in a functional assay or by quantitating the amount of protein present in a non-functional assay, e.g. immunostaining, ELISA, quantitation on Coomasie or silver stained gel, etc., and determining the ratio of biologically active protein to total protein.

Another method of measuring the amount of protein produced in coupled in vitro transcription and translation reactions is to perform the reactions using a known quantity of radiolabeled amino acid such as ³⁵S-methionine, ³H-leucine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

### EXAMPLES

As used herein, the symbols and conventions used in these processes, schemes and examples, regardless of whether a particular abbreviation is specifically defined, are consistent with those used in the contemporary scientific literature, for example, the Journal of Biological Chemistry.

For all of the following examples, standard work-up and purification methods known to those skilled in the art can be utilized. Unless otherwise indicated, all temperatures are expressed in ° C. (degrees Centigrade). All methods are conducted at room temperature unless otherwise noted.

### Example 1: Identifying Modified Antibodies for Optimal Stability and Conjugation

In this example, several IgG1 HC LQR variants were developed and tested as transglutaminase conjugation substrates with higher conjugation efficiency than parent antibody molecule.

First, the crystal structure of IgG (PDB 1HZH) was analyzed to select beneficial positions for LQR insertion. The positions were selected in flexible loops with exposed residues in constant regions of the heavy chain and light chain. The loops between constant domains CH₂ and CH₃ were avoided because mutations at these sites might affect antibody properties.

Second, the IgG HC-Q295 was mutated to A to avoid parent IgG Q conjugation. LQR variants were constructed for Trastuzumab Light Chain and aFolR1_1848-B10_Hc_Q295A heavy chain and subcloned into a pUG expression vector.

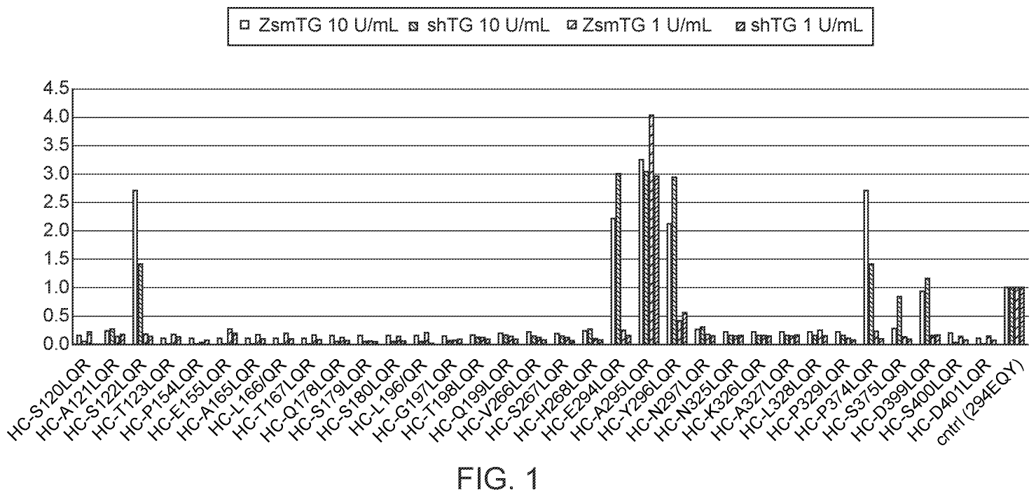

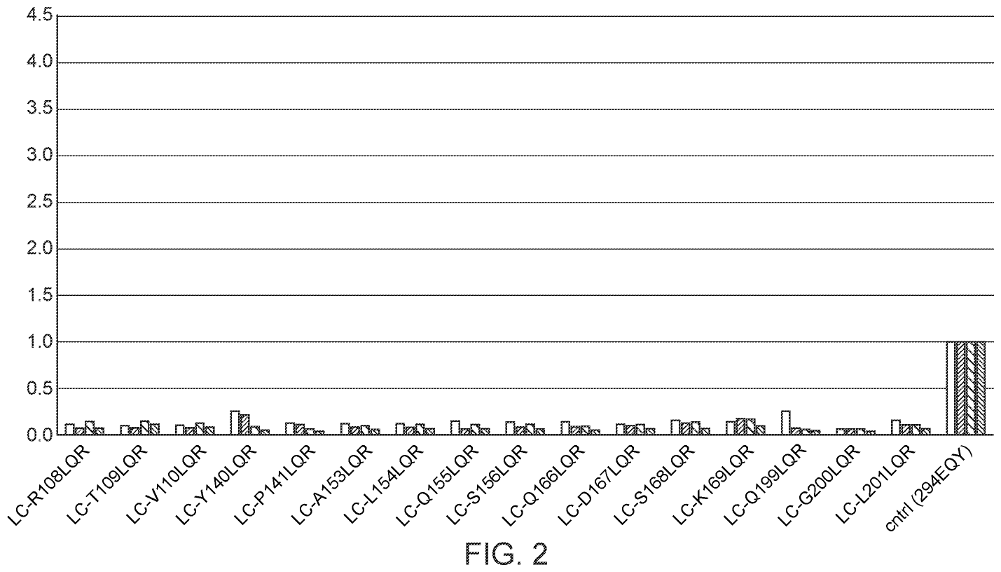

The LQR mutants were synthesized using cell-free technology by co-expressing aFolR1_1848-B10_Hc_Q295A and Trastuzumab LC. The parent and mutant antibody molecules were purified and reacted with TAMRA-cadaverine, which was catalyzed by ZsmTG, a transglutaminase of Streptomyces mobrensis (Zedria GmbH). These IgG variants were also catalyzed by shTG, a transglutaminase of Streptomyces hydroscopicus produced recombinantly in house. The final conjugation efficiency of IgG variants were analyzed by SDS-PAGE and quantified by fluorescence intensity. The normalized fluorescence intensity catalyzed by ZsmTG and shTG at 10 U/ml and 1 U/ml respectively are shown in FIG. 1 (heavy chains) and FIG. 2 (light chains).

Six HC sites showed appreciable Gln conjugation activity, while all LC sites were inactive. Three LQR variants (HC-E294LQR, A295LQR & Y296LQR) are clustered around the native Q295 conjugation site. The HC-E295LQR sequence has higher fluorescence intensity than the native HC sequence. Three other locations showed activity as TGase substrates include HC-S122LQR, HC-P374LQR, and HC-D399LQR. These mutants are promising candidate molecules for conjugation.

### Example 2: Simultaneous Transglutaminase and Click Conjugation

This example demonstrates transglutaminase conjugation at mutant LQR sequences.

Dual conjugation of the transglutaminase (TGase) active primary amine-linker-TLR 7/8 agonist drug (Compound 1002) and DBCO-linker-warhead (Compound 101) were performed in a single-step reaction.

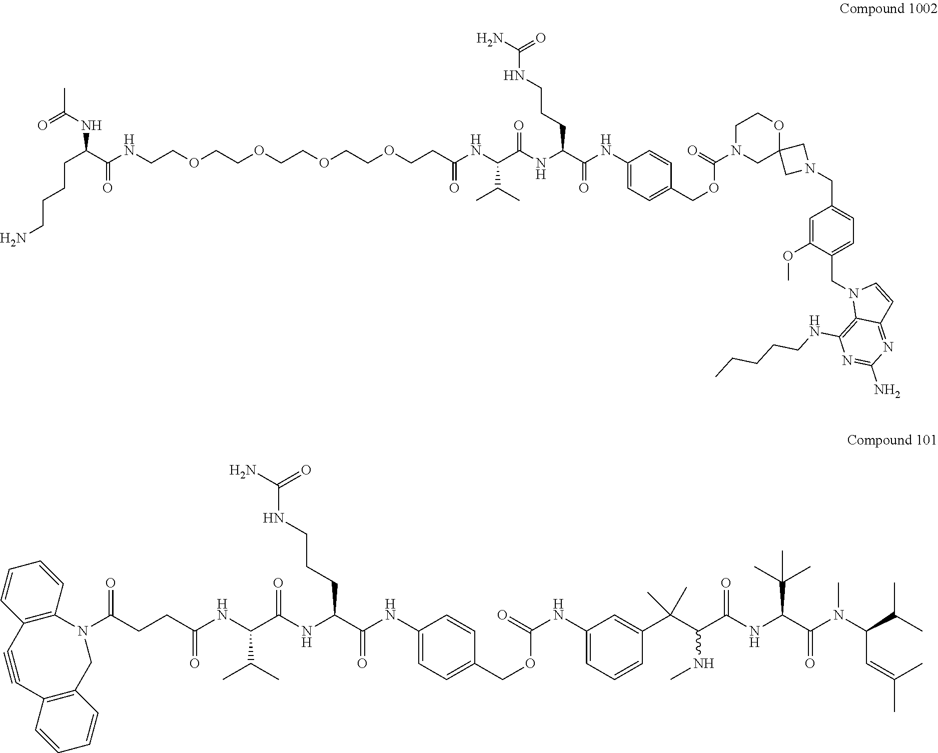 [SMILES: CCCCCNc1nc(N)nc2ccn(Cc3ccc(CN4CC5(C4)CN(C(=O)OCc4ccc(NC(=O)[C@H](CCCNC(N)=O)NC(=O)[C@@H](NC(=O)CCOCCOCCOCCOCCNC(=O)[C@@H](CCCCN)NC(C)=O)C(C)C)cc4)CCO5)cc3OC)c12 ; CNC(C(=O)N[C@H](C(=O)N(C)[C@H](C=C(C)C)C(C)C)C(C)(C)C)C(C)(C)c1cccc(NC(=O)OCc2ccc(NC(=O)[C@H](CCCNC(N)=O)NC(=O)[C@@H](NC(=O)CCC(=O)N3Cc4ccccc4C#Cc4ccccc43)C(C)C)cc2)c1]

The DBCO-Azide SPAAC reaction was performed using the p-azido-methyl-phenylalanine (pAMF) non-natural amino acid incorporated into the protein at LC position K42 and HC position Y180 (Kabat) to give an overall drug antibody ratio (DAR) of 4 for Compound 101. The transglutaminase reaction was carried out at HC position Y296 by introducing the mutations Q295L/Y296Q/N297R into the protein sequence to give an overall DAR of 2 for Compound 1002.

The conjugation reaction was performed with 1 mg/mL mAb, 0.1 U/mL TGase (Zedria GmbH, T001), 67 μM SC664, 80 μM SC239, 10 mM EDTA, and 100 mM Tris HCl, pH 8.0. The conjugation reaction was carried out for 16 hours at 37′° C. Unreacted drug and TGase enzyme were removed by preparative size exclusion chromatography (Superdex 200 Increase 10/300 GL, GE Life Sciences, 28990944) and the conjugate was stored in 10 mM Citrate, 9% Sucrose, pH 6.0. DAR analysis was performed by LCMS (Agilent Technologies 6520 Accurate-Mass Q-TOF LC/MS).

### Example 3: C-Term Transglutaminase Tag Library Design and Screening

A library was designed to screen for TGase conjugation to genetically encoded peptide tags appended to the carboxy terminus of trastuzumab LC. This library was based around mutating the leucine residues at the −1 and −2 positions upstream of the acceptor glutamine residue in the TG consensus sequence of LLQG (Ota et al., (1999) Biopolymers, 50:193-200, and Strop et al., (2013) Chem. Biol., 20:161-167) and to identify TGase acceptor sequences with improved performance. A C-term LC library was constructed by appending the sequences -GGSXLQGPP or -GGSLXQGPP to the LC, where X is any of the 19 natural amino acids other than cysteine.

C-term library variants were synthesized using cell-free technology by co-expressing an anti-folate receptor antibody heavy chain (HC) with a Q295A mutation and trastuzumab LC with a C-terminal transglutaminase tag. The antibody molecules were purified and reacted with TAMRA-cadaverine, which was catalyzed by ZsmTG, a transglutaminase of Streptomyces mobrensis (Zedria GmbH). These IgG variants were also catalyzed by shTG, a transglutaminase of Streptomyces hydroscopicus produced recombinantly.

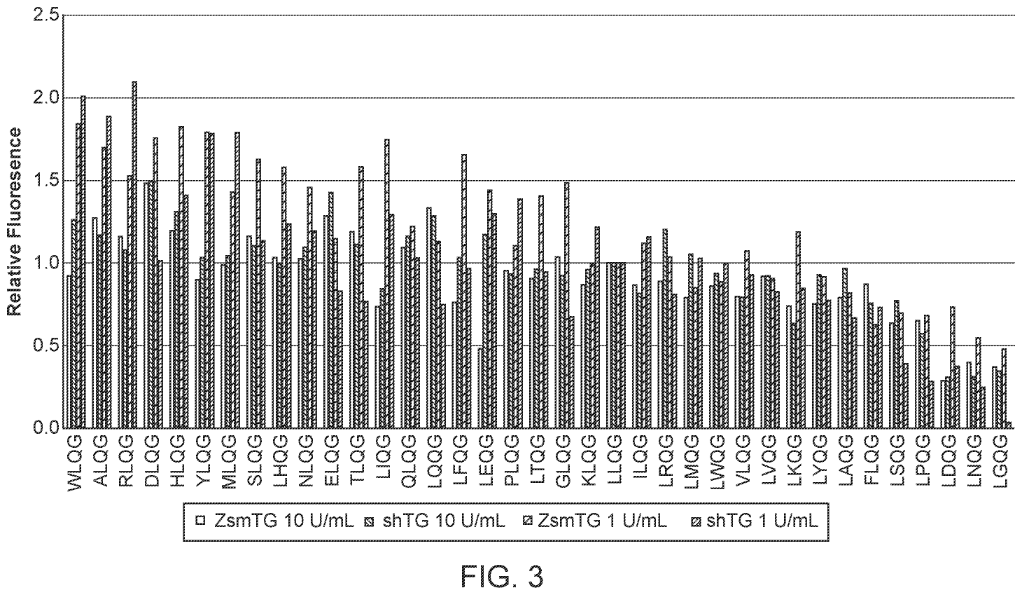

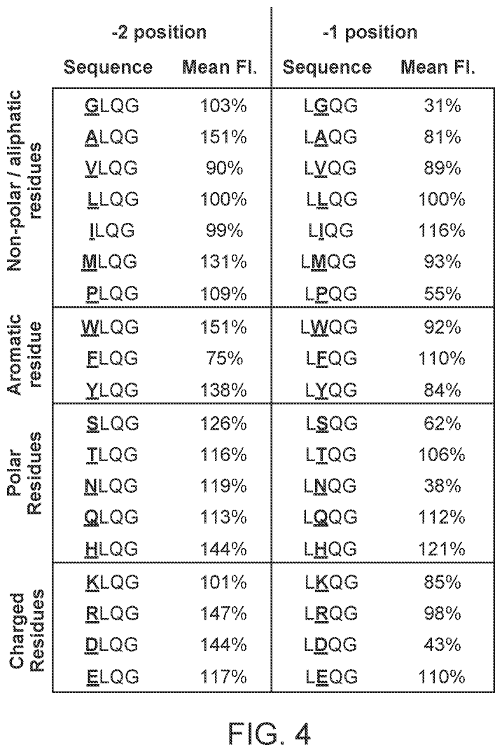

The final conjugation efficiency of IgG variants were analyzed by SDS-PAGE and quantified by fluorescence intensity. The normalized fluorescence intensities catalyzed by ZsmTG and shTG at 10 U/ml and 1 U/ml respectively are shown in FIG. 3. FIG. 4 shows the mean fluorescence intensity of the transglutaminase tag library members sorted by residue type and position. The −2 position is more accommodating to sequence variation than the −1 position. Several sequences identified in the −2 sub-library have greater fluorescence intensity than the LLQR consensus sequence. The sequences measured resulted in a fluorescence intensity of between 75-151% in comparison to the reference LLQG consensus sequence. Less sequence variation is tolerated in the −1 position however, with a fluorescence intensity that is 31-121% of that of the reference LLQG consensus sequence. See, FIG. 4. Surprisingly, at the −2 position, the amino acid residues G, A, I, M, P, W, Y, S, T, N, Q, H, K, R, D, and E showed similar or even greater activity compared to the consensus. Also surprisingly, at the −1 position, the amino acid residues I, F, T, Q, H, R and E showed similar or even greater activity compared to the consensus.

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

One or more features from any embodiments described herein or in the figures may be combined with one or more features of any other embodiments described herein or in the figures without departing from the scope of the invention.

All publications and patent applications cited in this specification are herein incorporated by reference as if each individual publication or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter limited solely by the scope of the following claims, including equivalents thereof.

Sequence Listing

1848-B10_Q295A
MEVQLVESGGGLVQPGGSLRLSCAASGFNTTTKSIHWVRQAPGKGLEWVG
EIYPRDGITDYADSVKGRFTISADTSKNTAYLQMNSLRAEDTAVYYCARG
GWHWRSGYSYYLDYWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHTCPPCPAPELLGGPSVFL
FPPKPKDTLMISRTPEVTCVVVDVSHEDPEVKFNWYVDGVEVHNAKTKPR
EEAYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK
Trastuzumab LC SerOpt
MDIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC

1. An antibody comprising one or more non-naturally occurring sequences of amino acids having:
the sequence leucine (L)-glutamine (Q)-arginine (R), wherein the Q residue of one, or up to all, of the non-naturally occurring sequences is at heavy chain site 295, 296, 297, 120, 375, or 400, when the heavy chain is numbered according to the Kabat or EU numbering scheme; or, alternatively, having:
the sequence X₁-leucine (L)-glutamine (Q)-glycine (G),
wherein X₁ is an amino acid selected from the group consisting of glycine (G), alanine (A), isoleucine (I), methionine (M), proline (P), tryptophan (W), tyrosine (Y)), serine (S), threonine (T), asparagine (N), glutamine (Q), histidine (H), lysine (K), arginine (R), aspartate (D), and glutamate (E), or
leucine (L)-X₂-glutamine (Q)-glycine (G),
wherein X₂ is an amino acid selected from the group consisting of isoleucine (I), phenylalanine (F), threonine (T), glutamine (Q), histidine (H), arginine (R), and glutamate (E),
wherein the non-naturally occurring amino acid sequence comprising X₁-L-Q-G or L-X₂-Q-G is at the C-terminus of an antibody light chain or an antibody heavy chain.

2. The antibody of claim 1 wherein the Q residue of one, up to all, of the non-naturally occurring sequences is capable of accepting a primary amine in a transglutaminase reaction.

3. The antibody of claim 1 comprising the sequence LQR at one or more of the following heavy chain sites, when the heavy chain is numbered according to the EU numbering scheme:
a. 294-295-296
b. 295-296-297
c. 296-297-298
d. 119-120-121
e. 374-375-376; and
f. 399-400-401.

4. The antibody of claim 1 comprising sequences LQR at two, three, four, five, or six of the heavy chain sites.

5. The antibody of claim 1 comprising the amino acid sequence X₁-L-Q-G or L-X₂-Q-G at the C-terminus of one or both light chains.

6. The antibody of claim 1 comprising the amino acid sequence X₁-L-Q-G or L-X₂-Q-G at the C-terminus of one or both heavy chains.

7. The antibody of claim 1 comprising the amino acid sequence X₁-L-Q-G or L-X₂-Q-G at the C-terminus of at least one heavy and at least one light chain.

8. The antibody of claim 5, comprising the amino acid sequence G-G-S-X₁-L-Q-G-P-P at the C-terminus of one or both light chains.

9.-40. (canceled)

41. The antibody of claim 5, comprising the amino acid sequence G-G-S-L-X₂-Q-G-P-P at the C-terminus of one or both light chains.

42. The antibody of claim 6, comprising the amino acid sequence G-G-S-X₁-L-Q-G-P-P at the C-terminus of one or both heavy chains.

43. The antibody of claim 6, comprising the amino acid sequence G-G-S-L-X₂-Q-G-P-P at the C-terminus of one or both heavy chains.

44. The antibody of claim 5, wherein X₁ is an amino acid selected from the group comprising G, A, I, M, P, W, Y, S, T, N, Q, H, K, R, D, and E.

45. The antibody of claim 5, wherein X₂ is an amino acid selected from the group comprising I, F, T, Q, H, R and E.

46. The antibody of claim 1, further comprising a non-natural amino acid residue at a specific site selected from the group consisting of optimally substitutable positions of any polypeptide chain of said antibody.

47. The antibody of claim 1, further comprising two or more site-specific non-natural amino acid residues.

48. The antibody of claim 1, further comprising two to six non-natural amino acid residues.

49. The antibody of claim 1, further comprising one or more site-specific non-natural amino acid residues at sequence positions corresponding to residues selected from the group consisting of consisting of heavy chain or light chain residues HC404, HC121, HC180, LC22, LC7, LC42, LC152, HC136, HC25, HC40, HC119, HC190, HC222, HC19, HC52, HC70, HC110, or HC221 according to the EU, Kabat or Chothia numbering scheme, or a post-translationally modified variant thereof.

50. The antibody of claim 49, wherein at least one of said non-natural amino acid residues is a para-azidophenylalanine or para-azidomethylphenylalanine residue.

51. The antibody of claim 1 comprising a heavy chain of a type selected from the group consisting of α, δ, ε and μ.

52. The antibody of claim 1 comprising a light chain of a type selected from λ and κ.

53. The antibody claim 1 that is of a class or subclass selected from the group consisting of IgA, IgA2, IgD, IgE, IgG, IgG1, IgG2, IgG3 and IgM.

54. The antibody of claim 1 that is in a form selected from the group consisting of Fv, Fc, Fab, (Fab′)₂, single chain Fv (scFv) and full-length antibody.

55. A antibody conjugate comprising the antibody of claim 1 linked to one or more therapeutic moieties or labeling moieties, directly or via a linker.

56. The antibody conjugate of claim 55 that comprises said antibody linked to one or more drugs or polymers.

57. The antibody conjugate of claim 55 that comprises said antibody linked to one or more labeling moieties.

58. The antibody conjugate of claim 55 that comprises said antibody linked to one or more single-chain binding domains (scFv).

59. The antibody conjugate of claim 55, wherein at least one of said therapeutic moieties or labeling moieties is linked to said antibody via a glutamine residue in an LQR, X₁-L-Q-G, or L-X₂-Q-G sequence.

60. The antibody conjugate of claim 55, wherein at least one of said therapeutic moieties or labeling moieties is linked to said antibody via a site-specific non-natural amino acid residue.

61. The antibody conjugate of claim 55, wherein at least one of said therapeutic moieties or labeling moieties is linked to said antibody via a site-specific para-methylphenylalanine or para-azidomethylphenylalanine residue.

62. The antibody conjugate of claim 55, wherein at least one of said therapeutic moieties or labeling moieties is linked to said antibody via a glutamine residue in an LQR, X₁-L-Q-G, or L-X₂-Q-G sequence, and at least one of said therapeutic moieties or labeling moieties is linked to said antibody via a site-specific para-methylphenylalanine or para-azidomethylphenylalanine residue.

63. A composition comprising the antibody or antibody conjugate of claim 1, wherein said antibody or antibody conjugate is at least 95% by mass of the total antibody or antibody conjugate mass of said composition.

64. A method of treating a subject in need thereof comprising administering to said subject an effective amount of a antibody conjugate of claim 55.

65. The method of claim 64, wherein said subject is afflicted with cancer.

66. The method of claim 65, wherein said cancer is breast cancer.

67. The method of claim 64, wherein the conjugate comprises at least one therapeutic moiety linked via a glutamine residue in an LQR, X₁-L-Q-G, or L-X₂-Q-G sequence, and at least a second therapeutic moiety linked via a site-specific para-methylphenylalanine or para-azidomethylphenylalanine residue.

68. The method of claim 64, wherein said antibody conjugate comprises a drug.

69. The method of claim 64, wherein said drug is a drug useful in the treatment of cancer.

70. The method of claim 64, wherein said drug is an auristatin, a maytansine, a hemiasterlin, or an immunostimulant.

71. The method of claim 64, wherein said drug is a TLR agonist.

72. The method of claim 64, wherein said antibody conjugate comprises a labeling moiety.

73. The method of claim 64, wherein said effective amount is a therapeutically effective amount.

74. A C-terminus light chain library comprising one or more antibody light chains having the amino acid sequence -GGSX₁LQGPP or -GGSLX₂QGPP at the carboxy terminus of said light chains, wherein X₁ or X₂ is any amino acid.

75. The library of claim 74, wherein X₁ or X₂ is any naturally occurring amino acid other than cysteine.

76. The library of claim 74, wherein X₁ or X₂ is any naturally occurring amino acid other than leucine.
